(19) United States
(12) Patent Application Publication
(10) Pub. No.: US 2024/0294611 A1
(43) Pub. Date: Sep. 5, 2024

(54) CROSS-REACTIVE ANTIBODIES RECOGNIZING THE CORONAVIRUS SPIKE S2 DOMAIN

(71) Applicants: Eduardo PADLAN, Kensington, MD (US); BOARD OF REGENTS, THE UNIVERSITY OF TEXAS SYSTEM, Austin, TX (US)

(72) Inventors: Jennifer MAYNARD, Austin, TX (US); Yimin HUANG, Austin, TX (US); Annalee NGUYEN, Austin, TX (US); Ching-Lin HSIEH, Austin, TX (US); Jason MCLELLAN, Austin, TX (US); Rui P. SILVA, Austin, TX (US); Edurado PADLAN, Kensington, MD (US)

(21) Appl. No.: 18/260,891

(22) PCT Filed: Jan. 11, 2022
(86) PCT No.: PCT/US2022/011906

§ 371 Date: Jul. 10, 2023

Related U.S. Application Data
(60) Provisional application No. 63/135,913, filed on Jan. 11, 2021.

(51) Int. Cl. C07K 16/10; C07K 14/005; G01N 33/569

(52) U.S. Cl. CPC C07K 16/1003; C07K 14/005; C07K 16/1002; G01N 33/56983; C07K 2317/24; C07K 2317/33; C07K 2317/76; C07K 2317/92; C12N 2770/20022; G01N 2333/165; G01N 2469/10; G01N 2469/20

(57) ABSTRACT

Provided herein are antibodies and antibody fragments that bind to the S2 domains of SARS-CoV-2, SARS-CoV, and MERS-CoV spike proteins. Methods of using these antibodies in in vitro methods are provided. Methods of using these antibodies in vivo to treat or prevent SARS-CoV-2 infections are also provided.

### REFERENCE TO RELATED APPLICATIONS

The present application claims the priority benefit of U.S. provisional application No. 63/234,776, filed Aug. 19, 2021, and U.S. provisional application No. 63/135,913, filed Jan. 11, 2021, the entire contents of each of which is incorporated herein by reference.

### STATEMENT REGARDING FEDERALLY SPONSORED RESEARCH

This invention was made with government support under Grant no. 2027066 awarded by the National Science Foundation. The government has certain rights in the invention.

### REFERENCE TO A SEQUENCE LISTING

The instant application contains a Sequence Listing, which has been submitted in ASCII format via EFS-Web and is hereby incorporated by reference in its entirety. Said ASCII copy, created on Jan. 7, 2022, is named UTFBP1269WO_ST25.txt and is 95,694 bytes in size.

### BACKGROUND

### 1. Field

The present disclosure relates generally to the fields of medicine, immunology, and virology. More particularly, it concerns cross-reactive antibodies that bind to the S2 domain of coronavirus spike proteins.

### 2. Description of Related Art

COVID-19, the disease caused by severe acute respiratory syndrome coronavirus 2 (SARS-CoV-2), has been responsible for over 3.5 million deaths worldwide since it was identified in late 2019. This pandemic is the latest and largest of three deadly coronavirus outbreaks including those caused by SARS-CoV-1 (SARS-CoV) in 2002 and Middle Eastern Respiratory Syndrome coronavirus (MERS-CoV) in 2012. Although COVID-19 has a much lower case fatality rate, estimated at ˜1-3% versus ˜10% for SARS-CoV and ˜34% for MERS (Abdelrahman et al., 2020), it has proven to be far more infectious and its spread has impacted every aspect of society worldwide. Four other coronaviruses are known to infect humans, resulting in relatively mild upper respiratory disease and symptoms: 229E, OC43 (both discovered in the 1960s), NL63 (2004), and HKU1 (2005). All seven of these coronaviruses are zoonotic and a large number of other coronaviruses are endemic in animals, foreshadowing future coronavirus outbreaks.

Coronaviruses are enveloped positive sense, single-stranded RNA viruses that invade target cells by fusion of the viral envelope with the target cell membrane, mediated by the spike glycoprotein. The spike is a homo-trimer comprised of the S1 and S2 domains, with S2 proximal to the viral envelope forming a stalk-like structure and S1 forming a cap over the end of S2. Each S1 domain monomer contains an N-terminal domain (NTD) and a receptor binding domain (RBD) that overlaps adjacent NTDs and RBDs within the trimer, forming a responsive surface that allows each RBD to extend to the “up” position for binding of the target receptor or tuck into the “down” position for immune shielding. When all three RBDs engage a receptor in the up position and the target-cell-anchored proteases prime the spike, the S1 domain is released from S2, propelling the fusion peptide into the target cell surface. Simultaneously, S2 undergoes a massive structural rearrangement to bring the viral envelope into contact with the target cell, initiating fusion and leaving spike in the post-fusion state (Cai et al., 2020).

A powerful strategy to prevent coronavirus fusion is to disrupt the interaction between the RBD and its host cell receptor. For SARS-CoV-2, the receptor is angiotensin-converting enzyme 2 (ACE2), and antibodies that block RBD binding to ACE2 are potently neutralizing and common in convalescent patient serum (Yuan et al., 2020). Accordingly, most monoclonal antibody therapies in development target this interaction (Renn et al., 2020). However, RBD sequences from different coronaviruses are quite different to allow for binding to a variety of host cell receptors. Even within the three human relevant coronaviruses that bind ACE2 (SARS-CoV, SARS-CoV-2 and NL63), RBD-binding antibodies exhibit limited cross-reactivity (Ly et al., 2020), consistent with the low level of S1 sequence conservation (˜20-24% identity, ˜41-52% similarity; FIGS. 8A-8D). As a result, it has been difficult to identify antibodies binding multiple lineage B β-coronaviruses, such as SARS-CoV and SARS-CoV-2 (Rappazzo et al., 2021), much less across lineages to include the lineage C MERS-CoV. In the CoV-AbDab database (Raybould et al., 2021) there were only two antibodies reported to neutralize SARS-CoV, SARS-CoV-2 and MERS-CoV and neither bind the S1 domain.

By contrast, the spike S2 domain is the most conserved spike domain, with 63-98% sequence similarity in pair-wise comparisons across the seven human coronaviruses (FIGS. 8A-8D). Moreover, the functionally analogous domain in influenza, respiratory syncytial virus (RSV) and human immunodeficiency virus (HIV) contains potently neutralizing epitopes (Impagliazzo et al., 2015; Corti et al., 2017), prompting speculation that the spike S2 domain may also be an effective target for neutralizing antibodies. Support for this position includes observations that antibodies binding the S2 subunit can synergize with antibodies blocking receptor binding and together prevent the emergence of MERS viral escape mutants (Wang et al., 2018). Multiple potential mechanisms inhibit viral fusion by targeting the S2 domain, including preventing S2 conformational rearrangement (Pinto et al., 2021; Sauer et al., 2021), blocking the fusion peptide (Channappanavar et al., 2015; Lu et al., 2014; Poh et al., 2020), or interfering with S2 proteolytic processing (Lu et al., 2020). Strategies targeting the S2 domain aim to induce premature S1 shedding and non-productive transformation into the post-fusion spike. Antibodies binding the S2 domain of SARS-CoV-2 or conserved epitopes on S2 are critical to support these efforts, but few have been reported in detail with structures limited to peptide-antibody complexes of two binding the membrane-proximal stem helix of the spike (Pinto et al.; 2021; Sauer et al., 2021). Of the over 2500 anti-SARS-CoV-2 spike antibodies reported in the CoV-AbDab database, less than 3% are reported to bind both SARS-CoV and SARS-CoV-2 spike and less than 10% bind the S2 region. A total of 13 S2 binding antibodies have neutralizing capacity (Raybould et al., 2021). There remains a need for antibodies that bind the S2 domain of SARS-CoV-2 or conserved epitopes on S2.

### SUMMARY

Here, antibodies binding epitopes conserved across all known highly pathogenic coronavirus strains were identified. To focus immune responses on the S2 domain, mice were immunized with a stabilized MERS-CoV S2 protein and antibodies isolated using alternate rounds of selection with the MERS-CoV S2 and intact SARS-CoV-2 spike using phage display. Of three high affinity, cross-reactive antibodies characterized, 3A3 neutralizes SARS-CoV-2 spike in cell fusion and pseudovirus assays by binding a conformational epitope at the apex of the S2 domain. This epitope is only accessible when the RBDs shift to the up position, as required for receptor binding and becomes increasingly accessible and vulnerable to 3A3 in spike variants that stabilize the RBD-up conformation in order to increase transmissibility. This epitope is also available for 3A3 binding when the spike protein is splayed into three C-terminally tethered protomers in a recently described spike conformation (Costello et al., 2021). The 3A3 epitope defines a novel site of vulnerability on spike, which is increasingly relevant for the more transmissible spike variants and may be an important target for future pan-coronavirus vaccines and therapeutic strategies.

In one embodiment, provided herein are monoclonal antibodies or antibody fragments comprising (a) a heavy chain variable region (VH) comprising VHCDR1, VHCDR2, and VHCDR3 amino acid sequences from the 3A3 antibody and a light chain variable region (VL) comprising VLCDR1, VLCDR2, and VLCDR3 amino acid sequences from the 3A3 antibody; (b) a heavy chain variable region (VH) comprising VHCDR1, VHCDR2, and VHCDR3 amino acid sequences from the AM3A3-3 antibody and a light chain variable region (VL) comprising VLCDR1, VLCDR2, and VLCDR3 amino acid sequences from the AM3A3-3 antibody; (c) a heavy chain variable region (VH) comprising VHCDR1, VHCDR2, and VHCDR3 amino acid sequences from the AM3A3-5 antibody and a light chain variable region (VL) comprising VLCDR1, VLCDR2, and VLCDR3 amino acid sequences from the AM3A3-5 antibody; (d) a heavy chain variable region (VH) comprising VHCDR1, VHCDR2, and VHCDR3 amino acid sequences from the AM3A3-3 antibody and a light chain variable region (VL) comprising VLCDR1, VLCDR2, and VLCDR3 amino acid sequences from the AM3A3-5 antibody; (e) a heavy chain variable region (VH) comprising VHCDR1, VHCDR2, and VHCDR3 amino acid sequences from the AM3A3-5 antibody and a light chain variable region (VL) comprising VLCDR1, VLCDR2, and VLCDR3 amino acid sequences from the AM3A3-3 antibody; (f) a heavy chain variable region (VH) comprising VHCDR1, VHCDR2, and VHCDR3 amino acid sequences from the 4H2 antibody and a light chain variable region (VL) comprising VLCDR1, VLCDR2, and VLCDR3 amino acid sequences from the 4H2 antibody; (g) a heavy chain variable region (VH) comprising VHCDR1, VHCDR2, and VHCDR3 amino acid sequences from the 4A5 antibody and a light chain variable region (VL) comprising VLCDR1, VLCDR2, and VLCDR3 amino acid sequences from the 4A5 antibody; or (h) a heavy chain variable region (VH) comprising VHCDR1, VHCDR2, and VHCDR3 amino acid sequences from the ven3A3 antibody and a light chain variable region (VL) comprising VLCDR1, VLCDR2, and VLCDR3 amino acid sequences from the AM3A3-5 antibody.

In one embodiment, provided herein are monoclonal antibodies or antibody fragments comprising (a) a heavy chain variable region (VH) comprising VHCDR1, VHCDR2, and VHCDR3 amino acid sequences derived from SEQ ID NO: 55 and a light chain variable region (VL) comprising VLCDR1, VLCDR2, and VLCDR3 amino acid sequences derived from SEQ ID NO: 56; (b) a heavy chain variable region (VH) comprising VHCDR1, VHCDR2, and VHCDR3 amino acid sequences derived from SEQ ID NO: 77 and a light chain variable region (VL) comprising VLCDR1, VLCDR2, and VLCDR3 amino acid sequences derived from SEQ ID NO: 78; (c) a heavy chain variable region (VH) comprising VHCDR1, VHCDR2, and VHCDR3 amino acid sequences derived from SEQ ID NO: 79 and a light chain variable region (VL) comprising VLCDR1, VLCDR2, and VLCDR3 amino acid sequences derived from SEQ ID NO: 80; (d) a heavy chain variable region (VH) comprising VHCDR1, VHCDR2, and VHCDR3 amino acid sequences derived from SEQ ID NO: 77 and a light chain variable region (VL) comprising VLCDR1, VLCDR2, and VLCDR3 amino acid sequences derived from SEQ ID NO: 80; (e) a heavy chain variable region (VH) comprising VHCDR1, VHCDR2, and VHCDR3 amino acid sequences derived from SEQ ID NO: 79 and a light chain variable region (VL) comprising VLCDR1, VLCDR2, and VLCDR3 amino acid sequences derived from SEQ ID NO: 78; (f) a heavy chain variable region (VH) comprising VHCDR1, VHCDR2, and VHCDR3 amino acid sequences derived from SEQ ID NO: 57 and a light chain variable region (VL) comprising VLCDR1, VLCDR2, and VLCDR3 amino acid sequences derived from SEQ ID NO: 58; (g) a heavy chain variable region (VH) comprising VHCDR1, VHCDR2, and VHCDR3 amino acid sequences derived from SEQ ID NO: 59 and a light chain variable region (VL) comprising VLCDR1, VLCDR2, and VLCDR3 amino acid sequences derived from SEQ ID NO: 60, or (h) a heavy chain variable region (VH) comprising VHCDR1, VHCDR2, and VHCDR3 amino acid sequences derived from SEQ ID NO: 55 or 68 and a light chain variable region (VL) comprising VLCDR1, VLCDR2, and VLCDR3 amino acid sequences derived from SEQ ID NO: 80. In some aspects, the antibodies or antibody fragments comprise clone-paired heavy and light chain CDR sequences from any of Tables 1-3.

In some aspects, the antibodies or antibody fragments comprise a VHCDR1 selected from SEQ ID NOs: 1, 19, and 37, a VHCDR2 selected from SEQ ID NOs: 2, 20, and 38, a VHCDR3 selected from SEQ ID NOs: 3, 21, and 39, a VLCDR1 selected from SEQ ID NOs: 4, 22, and 40, a VLCDR2 selected from SEQ ID NOs: 5, 23, and 41, and a VLCDR3 selected from SEQ ID NOs: 6, 24, and 42.

In some aspects, the antibodies or antibody fragments comprise a VHCDR1 selected from SEQ ID NOs: 7, 25, and 43, a VHCDR2 selected from SEQ ID NOs: 8, 26, and 44, a VHCDR3 selected from SEQ ID NOs: 9, 27, and 45, a VLCDR1 selected from SEQ ID NOs: 10, 28, and 46, a VLCDR2 selected from SEQ ID NOs: 11, 29, and 47, and a VLCDR3 selected from SEQ ID NOs: 12, 30, and 48.

In some aspects, the antibodies or antibody fragments comprise a VHCDR1 selected from SEQ ID NOs: 13, 31, and 49, a VHCDR2 selected from SEQ ID NOs: 14, 32, and 50, a VHCDR3 selected from SEQ ID NOs: 15, 33, and 51, a VLCDR1 selected from SEQ ID NOs: 16, 34, and 52, a VLCDR2 selected from SEQ ID NOs: 17, 35, and 53, and a VLCDR3 selected from SEQ ID NOs: 18, 36, and 54.

In some aspects, the antibodies or antibody fragments comprise a VHCDR1 selected from SEQ ID NOs: 81, 85, and 88, a VHCDR2 selected from SEQ ID NOs: 2, 20, and 38, a VHCDR3 selected from SEQ ID NOs: 82 and 89, a VLCDR1 selected from SEQ ID NOs: 83, 86, and 90, a VLCDR2 selected from SEQ ID NOs: 5, 23, and 41, and a VLCDR3 selected from SEQ ID NOs: 6, 24, and 42.

In some aspects, the antibodies or antibody fragments comprise a VHCDR1 selected from SEQ ID NOs: 1, 19, and 37, a VHCDR2 selected from SEQ ID NOs: 2, 20, and 38, a VHCDR3 selected from SEQ ID NOs: 3, 21, and 39, a VLCDR1 selected from SEQ ID NOs: 4, 22, and 40, a VLCDR2 selected from SEQ ID NOs: 5, 87, and 92, a VLCDR3 selected from SEQ ID NOs: 6, 24, and 42.

In some aspects, the antibodies or antibody fragments comprise a VHCDR1 selected from SEQ ID NOs: 81, 85, and 88, a VHCDR2 selected from SEQ ID NOs: 2, 20, and 38, a VHCDR3 selected from SEQ ID NOs: 82 and 89, a VLCDR1 selected from SEQ ID NOs: 4, 22, and 40, a VLCDR2 selected from SEQ ID NOs: 5, 87, and 92, a VLCDR3 selected from SEQ ID NOs: 6, 24, and 42.

In some aspects, the antibodies or antibody fragments comprise a VHCDR1 selected from SEQ ID NOs: 1, 19, and 37, a VHCDR2 selected from SEQ ID NOs: 2, 20, and 38, a VHCDR3 selected from SEQ ID NOs: 84 and 91, a VLCDR1 selected from SEQ ID NOs: 4, 22, and 40, a VLCDR2 selected from SEQ ID NOs: 5, 87, and 92, a VLCDR3 selected from SEQ ID NOs: 6, 24, and 42.

In some aspects, the antibodies or antibody fragments comprise clone-paired heavy chain and light chain variable sequences having, independently, at least 70%, 80%, or 90% identity to sequences from Tables 4, 6, and 7, optionally excluding the signal peptides. In some aspects, the antibodies or antibody fragments comprise clone-paired heavy chain and light chain variable sequences each having at least 95% identity to sequences from Tables 4, 6, and 7, optionally excluding the signal peptides. In some aspects, the antibodies or antibody fragments comprise clone-paired heavy chain and light chain variable sequences from Tables 4, 6, and 7, optionally excluding the signal peptides.

In some aspects, the antibodies or antibody fragments are humanized. In some aspects, the antibodies or antibody fragments comprise a heavy chain variable sequence and a light chain variable sequence each independently selected from Table 6 or 7 and having at least 70%, 80%, 90%, 95%, 96%, 97%, 98%, or 99% identity to the sequence from Table 6 or 7. In some aspects, the antibodies or antibody fragments comprise a heavy chain variable sequence and a light chain variable sequence each independently selected from Table 6 or 7. In some aspects, the antibodies or antibody fragments comprise a heavy chain variable sequence at least 70%, 80%, 90%, 95%, 96%, 97%, 98%, or 99% identical to SEQ ID NO: 68, 70, 72, 74, 77, or 79. In some aspects, the antibodies or antibody fragments comprise a heavy chain variable sequence of SEQ ID NO: 68, 70, 72, 74, 77, or 79. In some aspects, the antibodies or antibody fragments comprise a light chain variable sequence at least 70%, 80%, 90%, 95%, 96%, 97%, 98%, or 99% identical to SEQ ID NO: 69, 71, 73, 78, or 80. In some aspects, the antibodies or antibody fragments comprise a light chain variable sequence of SEQ ID NO: 69, 71, 73, 78, or 80. In some aspects, the antibody has a heavy chain variable sequence having a sequence according to SEQ ID NO: 68, or a heavy chain variable sequence having at least 70%, 80%, 90%, 95%, 96%, 97%, 98%, or 99% identity to SEQ ID NO: 68; and a light chain variable sequence having a sequence according to SEQ ID NO: 69, or a light chain variable sequence having at least 70%, 80%, 90%, 95%, 96%, 97%, 98%, or 99% identity to SEQ ID NO: 69. In some aspects, the antibody has a heavy chain variable sequence having a sequence according to SEQ ID NO: 68, or a heavy chain variable sequence having at least 70%, 80%, 90%, 95%, 96%, 97%, 98%, or 99% identity to SEQ ID NO: 68; and a light chain variable sequence having a sequence according to SEQ ID NO: 71, or a light chain variable sequence having at least 70%, 80%, 90%, 95%, 96%, 97%, 98%, or 99% identity to SEQ ID NO: 71. In some aspects, the antibody has a heavy chain variable sequence having a sequence according to SEQ ID NO: 68, or a heavy chain variable sequence having at least 70%, 80%, 90%, 95%, 96%, 97%, 98%, or 99% identity to SEQ ID NO: 68; and a light chain variable sequence having a sequence according to SEQ ID NO: 73, or a light chain variable sequence having at least 70%, 80%, 90%, 95%, 96%, 97%, 98%, or 99% identity to SEQ ID NO: 73.

In some aspects, the antibody has a heavy chain variable sequence having a sequence according to SEQ ID NO: 77, or a heavy chain variable sequence having at least 70%, 80%, 90%, 95%, 96%, 97%, 98%, or 99% identity to SEQ ID NO: 77; and a light chain variable sequence having a sequence according to SEQ ID NO: 78, or a light chain variable sequence having at least 70%, 80%, 90%, 95%, 96%, 97%, 98%, or 99% identity to SEQ ID NO: 78. In some aspects, the antibody has a heavy chain variable sequence having a sequence according to SEQ ID NO: 79, or a heavy chain variable sequence having at least 70%, 80%, 90%, 95%, 96%, 97%, 98%, or 99% identity to SEQ ID NO: 79; and a light chain variable sequence having a sequence according to SEQ ID NO: 78, or a light chain variable sequence having at least 70%, 80%, 90%, 95%, 96%, 97%, 98%, or 99% identity to SEQ ID NO: 78. In some aspects, the antibody has a heavy chain variable sequence having a sequence according to SEQ ID NO: 77, or a heavy chain variable sequence having at least 70%, 80%, 90%, 95%, 96%, 97%, 98%, or 99% identity to SEQ ID NO: 77; and a light chain variable sequence having a sequence according to SEQ ID NO: 80, or a light chain variable sequence having at least 70%, 80%, 90%, 95%, 96%, 97%, 98%, or 99% identity to SEQ ID NO: 80. In some aspects, the antibody has a heavy chain variable sequence having a sequence according to SEQ ID NO: 79, or a heavy chain variable sequence having at least 70%, 80%, 90%, 95%, 96%, 97%, 98%, or 99% identity to SEQ ID NO: 79; and a light chain variable sequence having a sequence according to SEQ ID NO: 80, or a light chain variable sequence having at least 70%, 80%, 90%, 95%, 96%, 97%, 98%, or 99% identity to SEQ ID NO: 80.

In some aspects, the antibody fragment is a monovalent scFv (single chain fragment variable) antibody, divalent scFv, Fab fragment, F(ab′)₂ fragment, F(ab′)₃ fragment, Fv fragment, or single chain antibody. In some aspects, the antibodies are chimeric antibodies, bispecific antibodies, or BiTEs. In some aspects, the antibodies are IgG antibodies. In some aspects, the antibodies or antibody fragments are recombinant IgG antibodies or antibody fragments. In some aspects, the Fab fragments comprise clone-paired heavy chain and light chain variable sequences having, independently, at least 70%, 80%, or 90% identity to sequences from Table 5, optionally excluding the signal peptides. In some aspects, the Fab fragments comprise clone-paired heavy chain and light chain variable sequences each having at least 95% identity to sequences from Table 5, optionally excluding the signal peptides. In some aspects, the Fab fragments comprise clone-paired heavy chain and light chain variable sequences from Table 5, optionally excluding the signal peptides.

In some aspects, the antibodies or antibody fragments are capable of binding to a coronavirus spike protein. In some aspects, the coronavirus spike protein is from SARS-CoV, SARS-CoV-2, or MERS. In some aspects, the antibodies or antibody fragments are capable of binding to the spike proteins from SARS-CoV, SARS-CoV-2, and MERS.

In some aspects, the antibodies or antibody fragments are fused to imaging agents. In some aspects, the antibodies or antibody fragments are labeled. In some aspects, the label is a fluorescent label, an enzymatic label, or a radioactive label.

In one embodiment, provided herein are monoclonal antibodies or antibody fragments, which compete for binding to the same epitope of a coronavirus spike protein as the monoclonal antibodies or antibody fragments according to any one of the present embodiments.

In one embodiment, provided herein are monoclonal antibodies or antibody fragments that bind to an epitope on a coronavirus spike protein recognized by the monoclonal antibodies or antibody fragments of any one of the present embodiments. In some aspects, the epitope is the apex of a coronavirus spike S2 domain. In some aspects, the epitope is present within a portion of a coronavirus spike protein that corresponds to amino acids 978-1006 of SEQ ID NO: 67. In some aspects, when bound to a coronavirus spike protein, the monoclonal antibody binds to at least one residue of a coronavirus spike protein that corresponds to position 980 (e.g., I980), 983 (e.g., R983), 984 (e.g., L984), 985 (e.g., D985), 992 (e.g., Q992), or 995 (e.g., R995) of SEQ ID NO: 67. In some aspects, when bound to a coronavirus spike protein, the monoclonal antibody binds to residues of a coronavirus spike protein that correspond to positions 980 (e.g., I980), 983 (e.g., R983), 984 (e.g., L984), 985 (e.g., D985), 992 (e.g., Q992), and 995 (e.g., R995) of SEQ ID NO: 67. In some aspects, when bound to a coronavirus spike protein, the monoclonal antibody additionally binds to at least one residue of a coronavirus spike protein the corresponds to position 987 (e.g., 1987, L987, or V987), 988 (e.g., Q988 or E988), or 990 (e.g., E990 or D990) of SEQ ID NO: 67. In some aspects, the monoclonal antibodies or antibody fragments are capable of binding to the spike proteins from SARS-CoV, SARS-CoV-2, and MERS.

In one embodiment, provided herein are isolated nucleic acids encoding the antibody heavy and/or light chain variable region of the antibody or antibody fragment of any one of the present embodiments. In one embodiment, provided herein are expression vectors comprising the nucleic acid of any one of the present embodiments. In one embodiment, provided herein are hybridomas or engineered cells comprising a nucleic acid encoding an antibody or antibody fragment of any one of the present embodiments. In one embodiment, provided herein are hybridomas or engineered cells comprising a nucleic acid of the present embodiments.

In one embodiment, provided herein are methods of making the monoclonal antibody or antibody fragment of any one of the present embodiments, the method comprising culturing the hybridoma or engineered cell of the present embodiments under conditions that allow expression of the antibody or antibody fragment and optionally isolating the antibody or antibody fragment from the culture.

In one embodiment, provided herein are pharmaceutical formulations comprising one or more antibody or antibody fragment of any one of the present embodiments. In one embodiment, provided herein are pharmaceutical formulations comprising one or more expression vector encoding a first antibody or antibody fragment of any one of the present embodiments.

In one embodiment, provided herein are methods of reducing the likelihood of a pathogenic coronavirus infection in a patient at risk of contracting the pathogenic coronavirus, the method comprising delivering to the patient an antibody or antibody fragment of any one of the present embodiments. In some aspects, the methods are further characterized as methods of preventing a SARS-CoV-2 infection in the patient. In some aspects, the patient has been exposed to a coronavirus. In some aspects, the antibodies or antibody fragments are delivered to the patient prior to infection or after infection. In some aspects, delivering comprises antibody or antibody fragment administration, or genetic delivery with an RNA or DNA sequence or vector encoding the antibodies or antibody fragments.

In one embodiment, provided herein are methods of treating a patient infected with a pathogenic coronavirus, the methods comprising delivering to the patient an antibody or antibody fragment of any one of the present embodiments. In some aspects, delivering comprises antibody or antibody fragment administration, or genetic delivery with an RNA or DNA sequence or vector encoding the antibodies or antibody fragments. In some aspects, the methods reduce the viral load in the patient. In some aspects, the methods further comprise administering at least a second antibody that binds to a distinct epitope relative to the first antibody or antibody fragment administered to the patient.

In one embodiment, provided herein are methods of detecting a coronavirus infection in a patient, the method comprising: (a) contacting a sample obtained from the patient with an antibody or antibody fragment of any one of the present embodiments; and (b) detecting the coronavirus in the sample by detecting binding of the antibody or antibody fragment to a coronavirus antigen in the sample. In some aspects, the sample is a body fluid. In some aspects, the sample is blood, sputum, tears, saliva, mucous or serum, semen, cervical or vaginal secretions, amniotic fluid, placental tissues, urine, exudate, transudate, tissue scrapings or feces. In some aspects, detecting comprises performing an ELISA, RIA, biolayer interferometry (BLI), lateral flow assay or Western blot. In some aspects, the methods further comprise performing steps (a) and (b) a second time and determining a change in coronavirus antigen levels as compared to the first assay. In some aspects, the coronavirus is an emerging coronavirus. In some aspects, the coronavirus has not yet been identified at the time the method is performed.

In one embodiment, provided herein are methods of determining an antigenic integrity, correct conformation and/or correct sequence of a coronavirus spike protein, the method comprising: (a) contacting a sample comprising the coronavirus spike protein with a first antibody or antibody fragment of any one of the present embodiments; and (b) determining antigenic integrity, correct conformation and/or correct sequence of the coronavirus spike protein by detecting binding of the first antibody or antibody fragment to the antigen. In some aspects, the sample comprises a recombinantly produced coronavirus spike protein. In some aspects, the sample comprises a vaccine formulation comprising the coronavirus spike protein. In some aspects, detecting comprises performing an ELISA, RIA, biolayer interferometry (BLI), lateral flow assay or Western blot. In some aspects, the methods further comprise performing steps (a) and (b) a second time to determine the antigenic stability of the coronavirus spike protein over time.

In one embodiment, provided herein are methods of detecting a coronavirus spike protein in an in vitro sample, the method comprising contacting the in vitro sample with an antibody or antibody fragment of any one of the present embodiments and detecting the binding of the antibody or antibody fragment to the sample. In some aspects, the detecting is by flow cytometry, mass spectrometry, western blot, immunohistochemistry, ELISA, biolayer interferometry (BLI), or RIA.

In one embodiment, provided herein are antibodies or antibody fragments or pharmaceutical formulations of any of the present embodiments, for use in treating or preventing a coronavirus infection in a patient.

In one embodiment, provided herein are uses of antibodies or antibody fragments or pharmaceutical formulations of any of the present embodiments, in the manufacture of a medicament for treating or preventing a coronavirus infection in a patient.

In one embodiment, provided herein are engineered proteins that comprise a sequence at least 90% identical to: (a) positions 14-1208 of SEQ ID NO: 75 or 76; (b) positions 14-1160 of SEQ ID NO: 75 or 76; or (c) positions 319-1208 of SEQ ID NO: 75 or 76; wherein the engineered protein comprises the following substitutions relative to the sequence of SEQ ID NO: 75 or 76: F817P, A892P, A899P, A942P, K986P, V987P, and E1031R. In one embodiment, provided herein are engineered proteins that comprise a sequence at least 90% identical to: (a) positions 14-1208 of SEQ ID NO: 75 or 76; (b) positions 14-1160 of SEQ ID NO: 75 or 76; or (c) positions 319-1208 of SEQ ID NO: 75 or 76; wherein the engineered protein comprises the following substitutions relative to the sequence of SEQ ID NO: 1 or 2: S383C, F817P, A892P, A899P, A942P, D985C, K986P, and V987P. In some aspects, the engineered proteins have at least 95% identity to positions 319-1208 of SEQ ID NO: 75 or 76. In some aspects, the engineered proteins comprise an engineered coronavirus S protein ectodomain having 95% identity to positions 14-1208 of SEQ ID NO: 75 or 76.

In some aspects, the engineered proteins comprise the engineered coronavirus S protein ectodomain comprises a mutation that eliminates the furin cleavage site. In further aspects, the engineered proteins the mutation that eliminates the furin cleavage site comprises a GSAS substitution at positions 682-685.

Any of the substitutions described herein may engineered into any known coronavirus S protein variant, including, but not limited to, a coronavirus S protein having any one or more of the following modifications (see SEQ ID NO: 76): L5F, S13I, L18F, T19R, T20N, P26S, Q52R, A67V, H69del, V70del, V70I, D80A, T95I, D138Y, Y144del, Y144V, W152C, E154K, R190S, D215G, L242del, A243del, L244del, D253G, W258L, K417N, K417T, L452R, S477N, T478K, E484K, E484Q, E484K, N501Y, A570D, D614G, H655Y, Q677H, P681R, P681H, A701V, T7161, F888L, D950N, S982A, T10271, D1118H, and V1176F. Exemplary combinations of such modifications are provided in Table A.

In some aspects, the engineered proteins are fused or conjugated to a trimerization domain. In further aspects, the protein is fused to a trimerization domain. In some aspects, the a trimerization domain is positioned C-terminally relative to S protein ectodomain. In some aspects, the a trimerization domain comprises a T4 fibritin trimerization domain. In some aspects, the protein is fused or conjugated to a transmembrane domain. In some aspects, the protein is fused to a transmembrane domain. In some aspects, the transmembrane domain comprises a coronavirus spike protein transmembrane domain. In some aspects, the transmembrane domain comprises a SARS-CoV or a SARS-CoV-2 transmembrane domain.

In one embodiment, the present disclosure provides engineered coronavirus trimers comprising at least one subunit of the present disclosure. In one embodiment, the present disclosure provides nucleic acid molecules that encode an amino acid sequence of an engineered protein of the present disclosure. In one embodiment, the present disclosure provides compositions comprising an engineered protein of the present disclosure bound to an antibody.

In one embodiment, the present disclosure provides methods of detecting coronavirus S protein-binding antibodies in a sample or subject comprising: (a) contacting a subject or a sample from the subject with the an engineered S protein of the present disclosure; and (b) detecting binding of said antibody to the engineered S protein. In some aspects, the sample is a body fluid or biopsy. In some aspects, the sample is blood, bone marrow, sputum, tears, saliva, mucous, serum, urine, feces or a nasal swab. In some aspects, detection comprises immunohistochemistry, flow cytometry, FACS, ELISA, RIA or Western blot. In some aspects, the engineered S protein further comprises a label or a is immobilized on a surface. In further aspects, said label is a peptide tag, an enzyme, a magnetic particle, a chromophore, a fluorescent molecule, a chemo-luminescent molecule, or a dye. In some aspects, engineered S protein is conjugated to a liposome or nanoparticle.

In one embodiment, the present disclosure provides methods for detecting an antibody or antibody fragment that binds to an epitope on a coronavirus spike protein recognized by a monoclonal antibody or antibody fragment of the present disclosure. The method may comprise contacting a sample comprising an engineered protein of the present disclosure or the trimer thereof with an antibody or antibody fragment and detecting binding of the antibody or antibody fragment to the engineered protein or trimer. In some aspects, the sample comprises the 3A3 antibody or a fragment of the 3A3 antibody, and the method detects binding of the antibody or antibody fragment to the engineered protein or trimer to determine if the antibody or antibody fragment competes with 3A3 for binding to the engineered protein or trimer. In some aspects, the methods detect an antibody or antibody fragment capable of binding to the spike proteins from SARS-CoV, SARS-CoV-2, and MERS-CoV.

Other objects, features, and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1D. Antibodies 3A3, 4A5, and 4H2 bind to SARS-CoV and MERS-CoV spike proteins. (FIG. 1A) Full-length 3A3 (left), 4A5 (left/middle), 4H2 (right/middle), and 3E11 (right) were tested for binding to plates coated with SARS-CoV-2 (SARS-2), SARS-CoV-2 HexaPro (SARS-2 HP), SARS-CoV (SARS-1), MERS-CoV, HKU1, RSV F foldon, or milk (no coat) proteins by ELISA. (FIG. 1B) 4A5, 4H2, and 3E11 bind reduced, denatured SARS-2 HP, SARS-2, and MERS-CoV spike proteins by western blot, but 3A3 does not. No antibodies show binding to HKU1 spike protein by western blot.

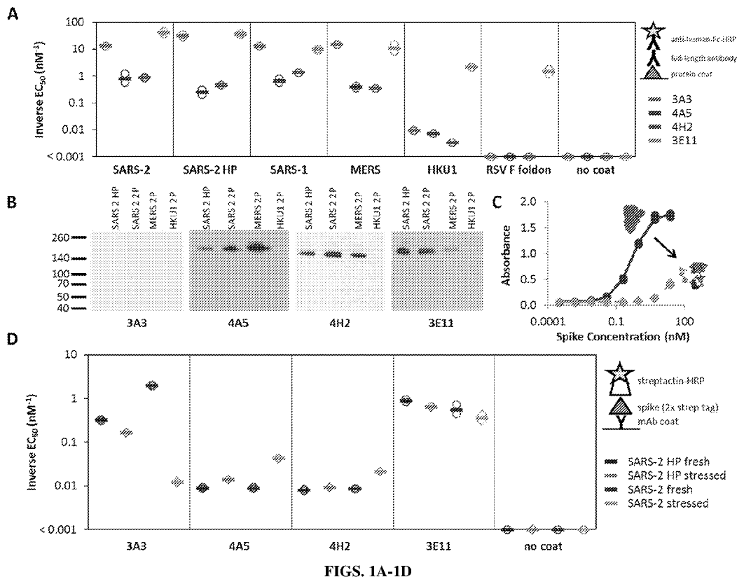

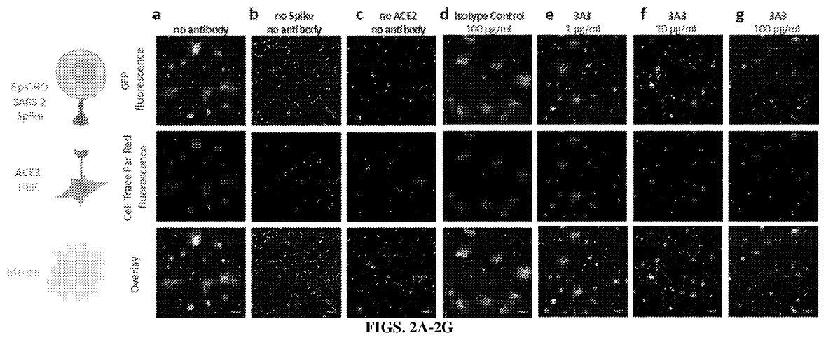

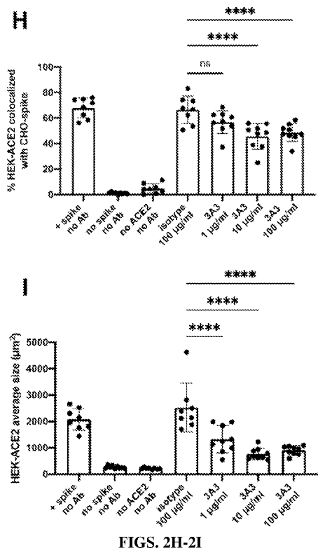

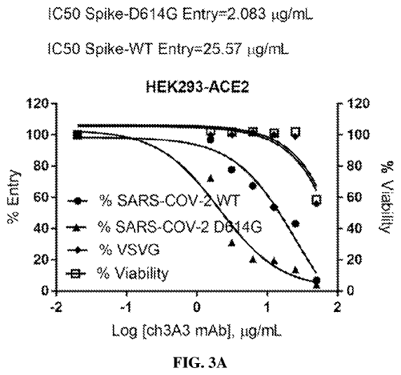

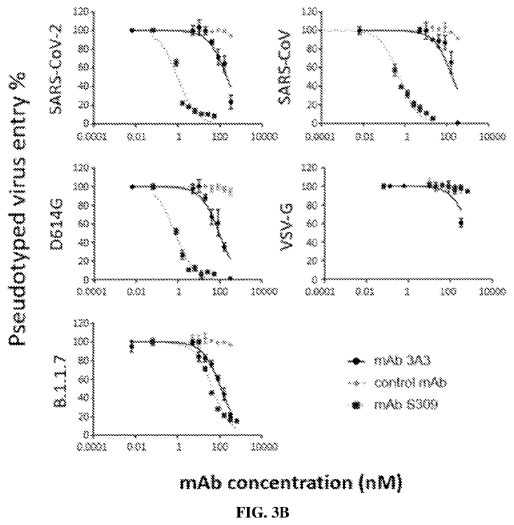

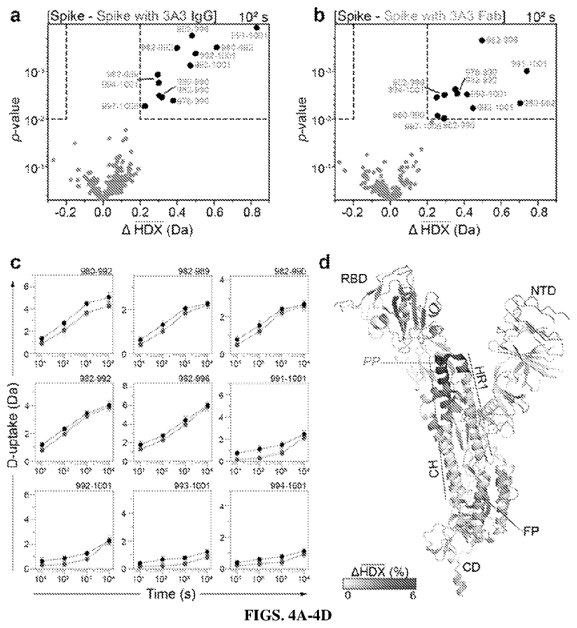

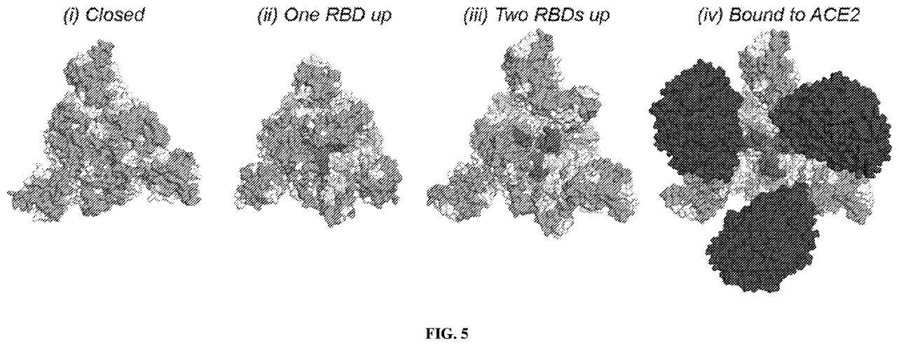

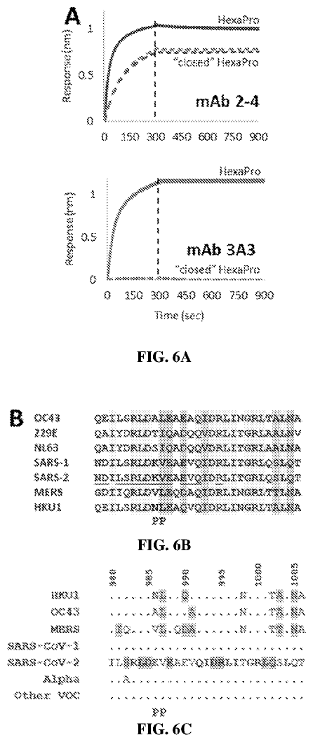

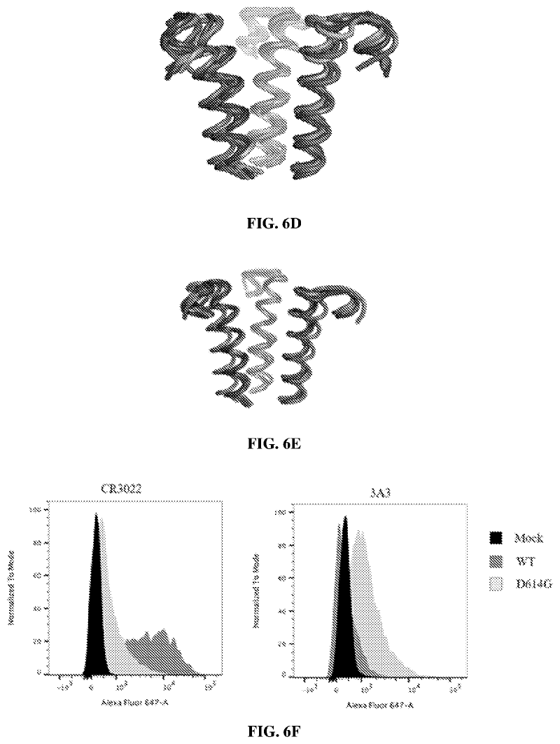

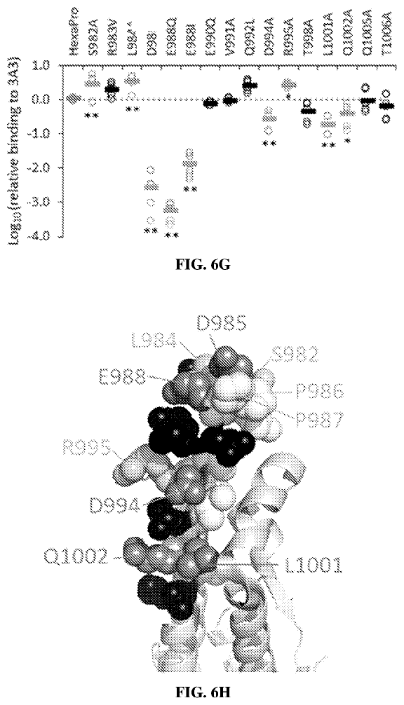

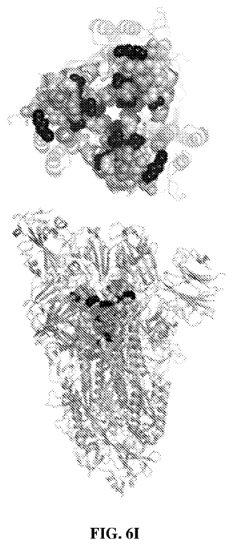

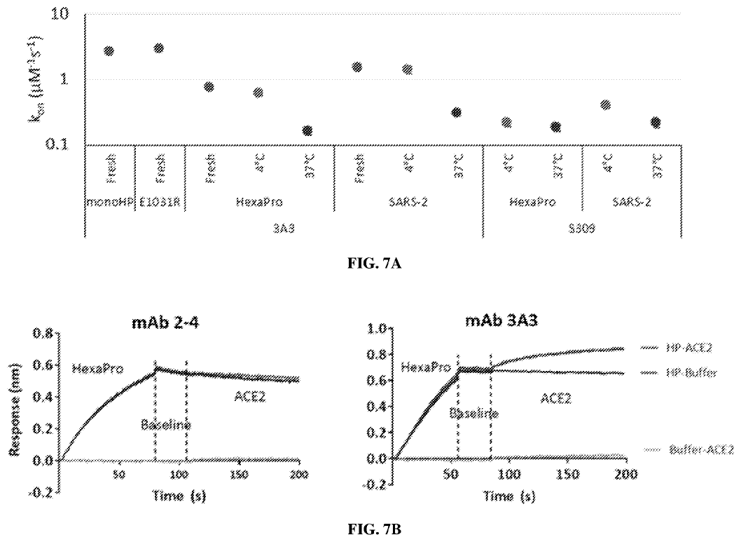

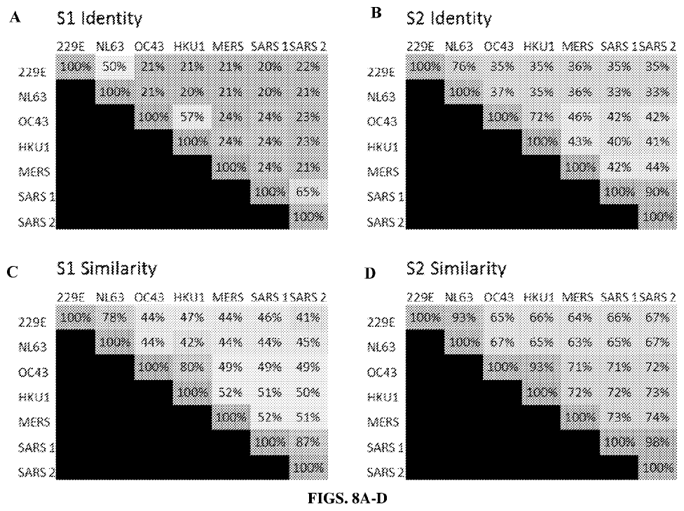

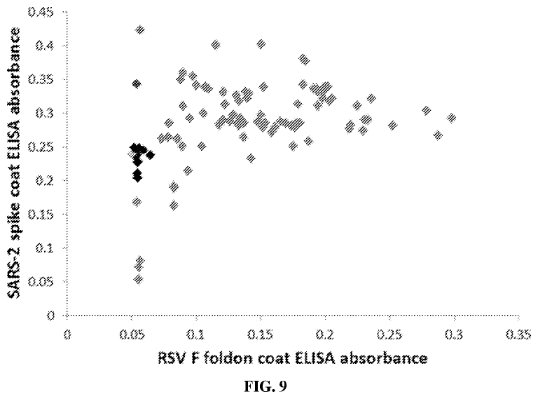

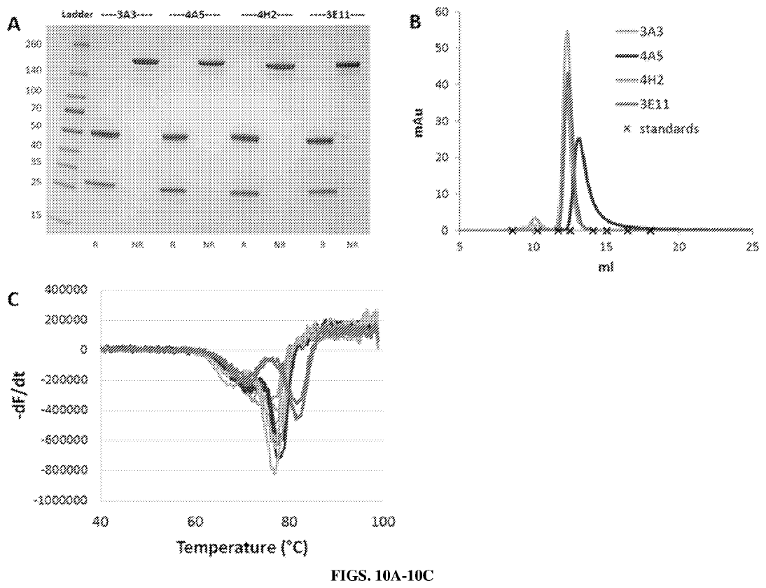

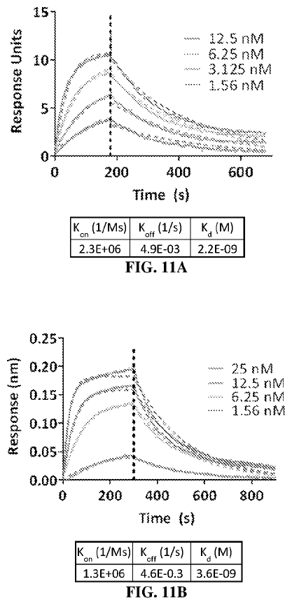

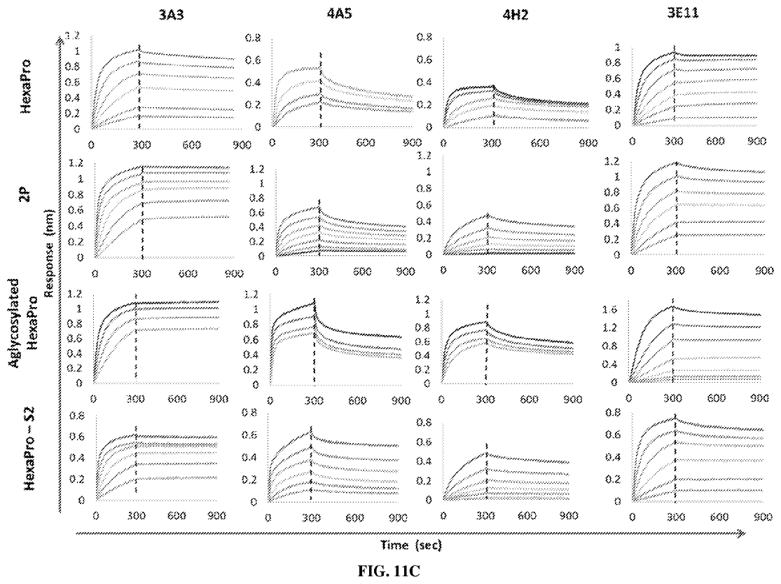

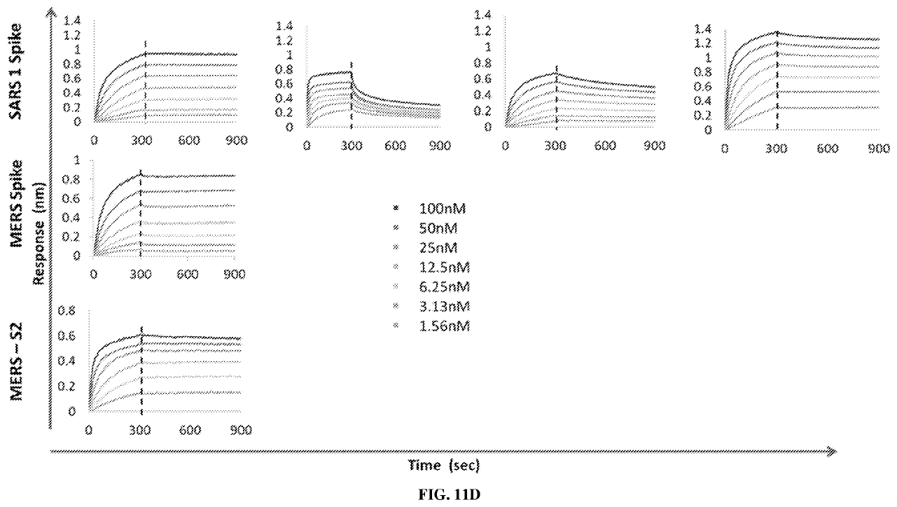

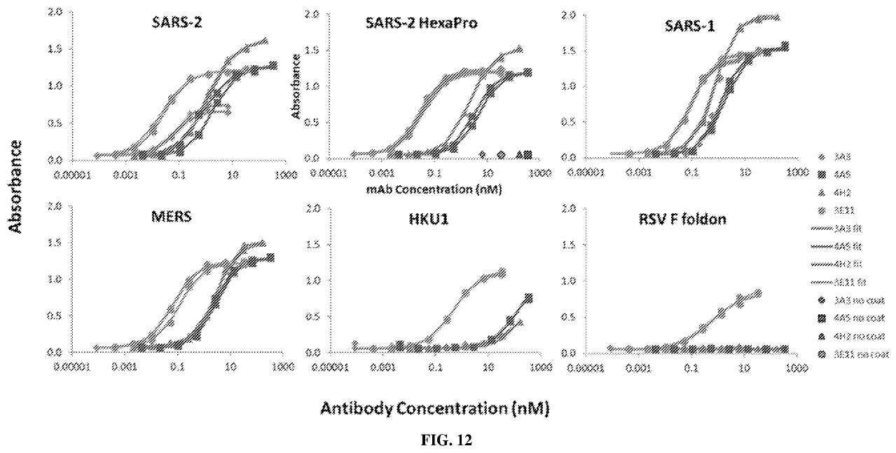

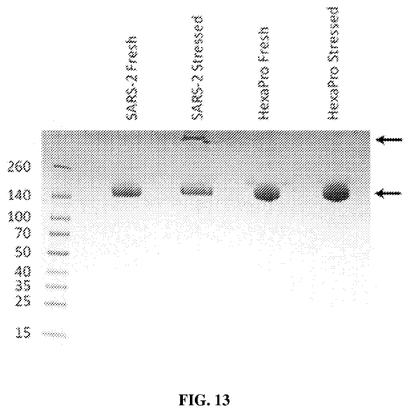

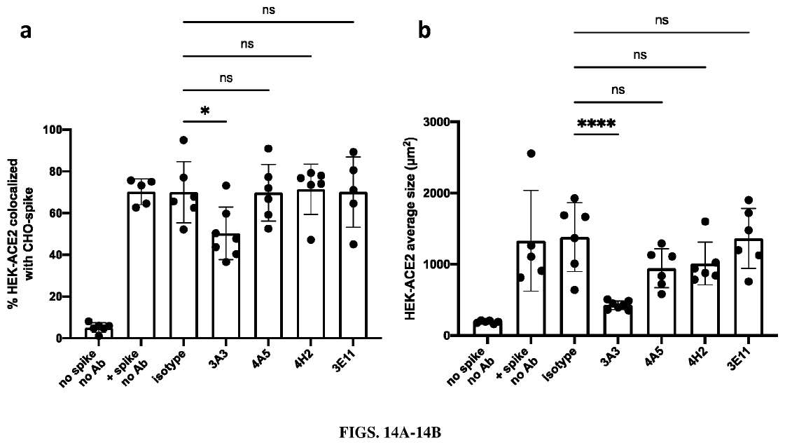

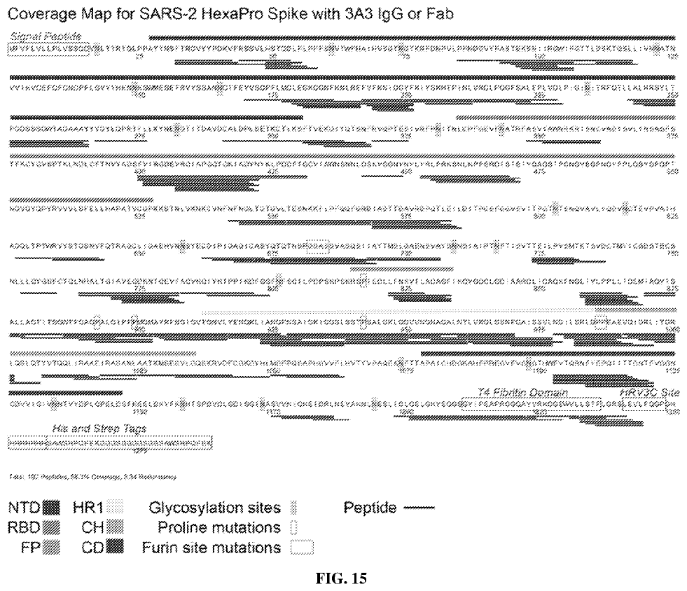

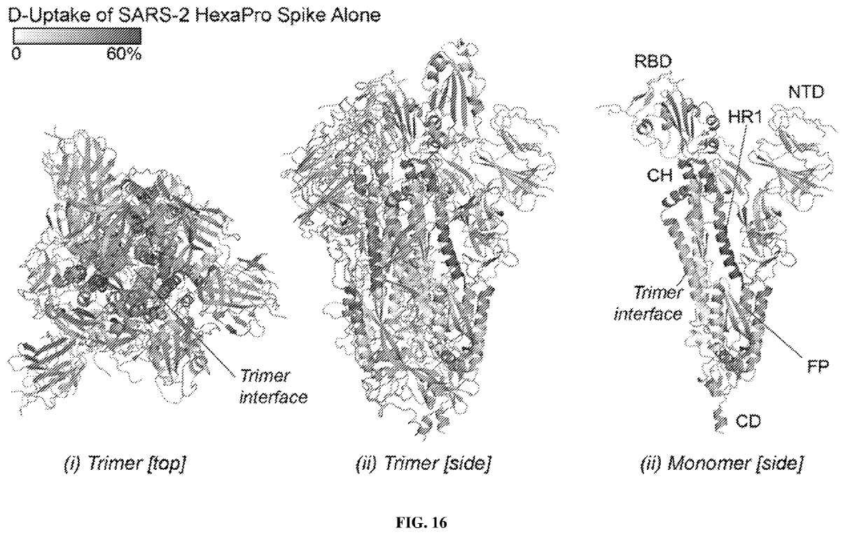

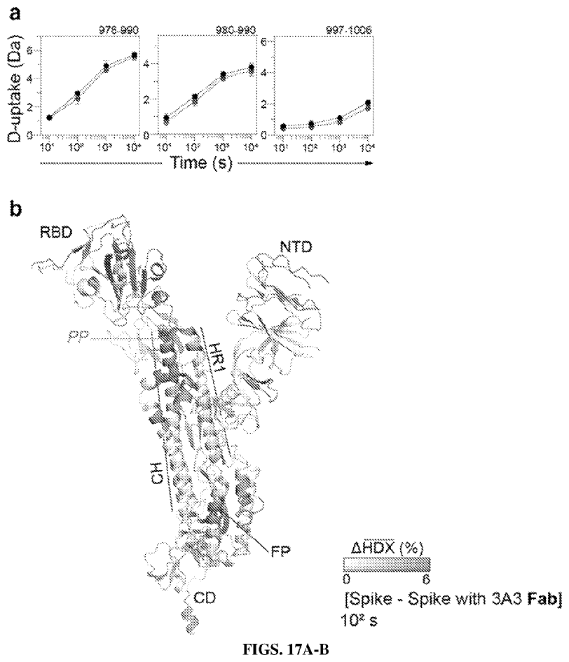

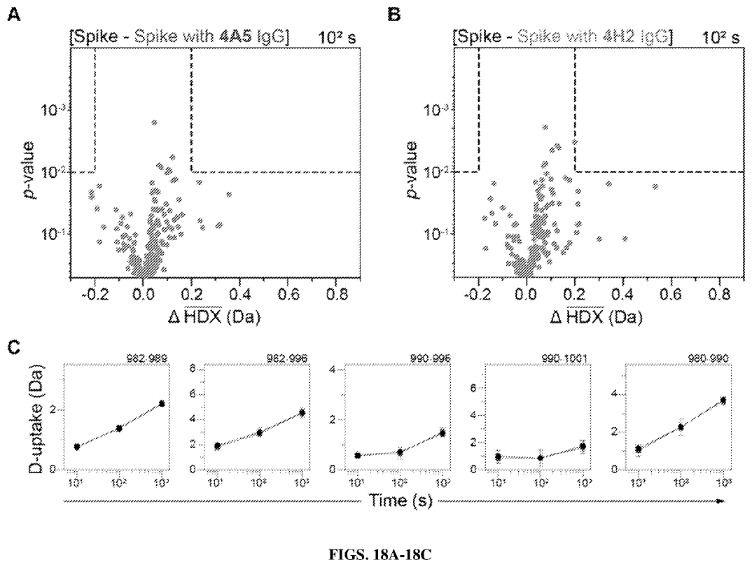

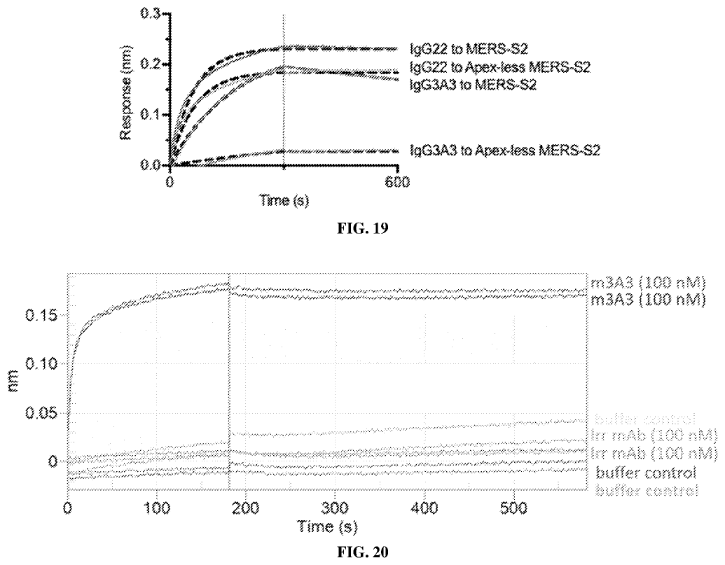

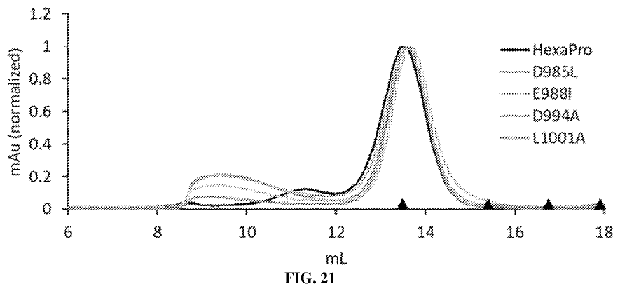

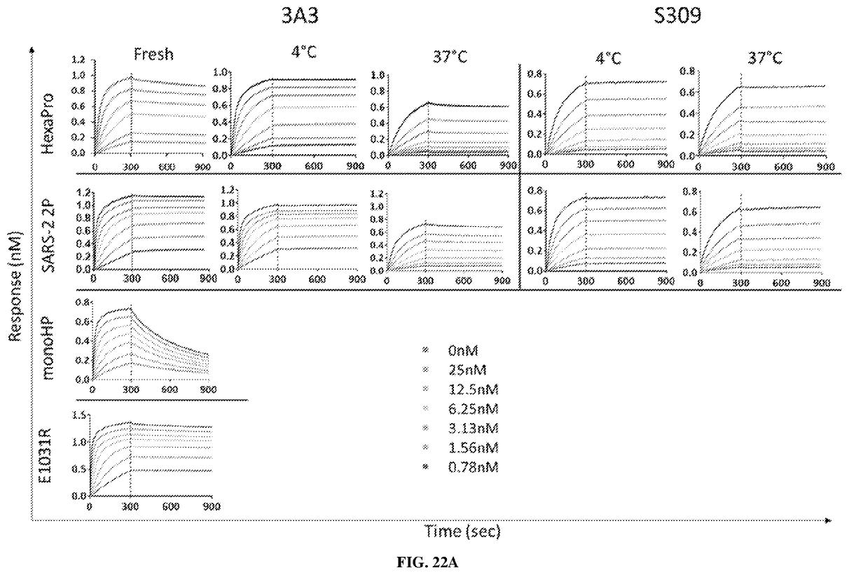

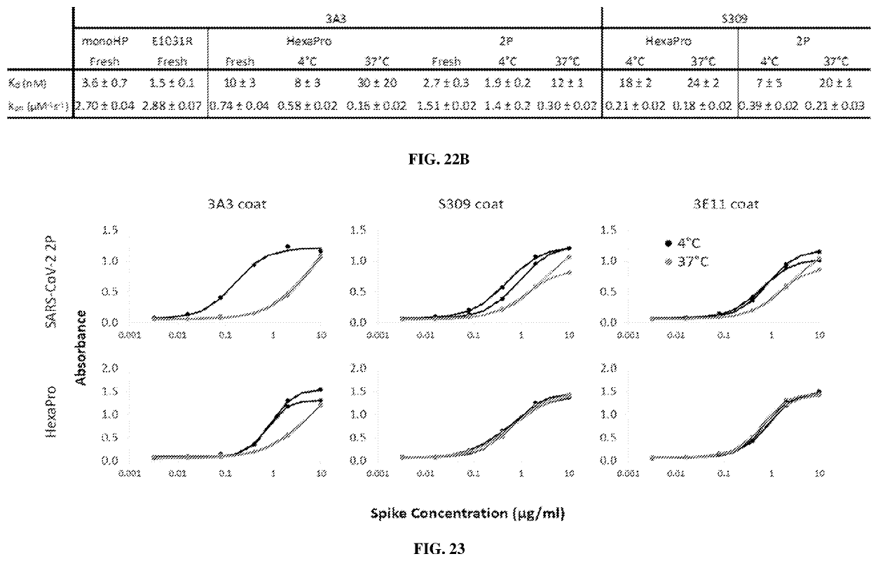

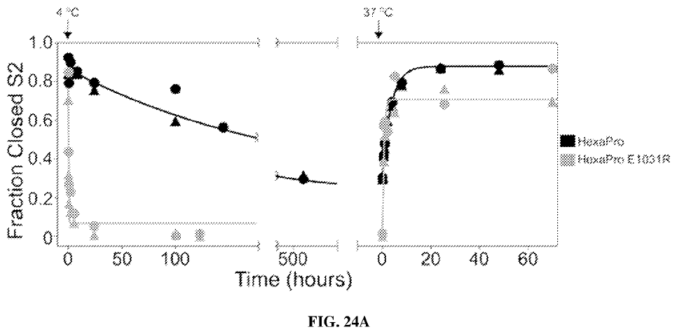

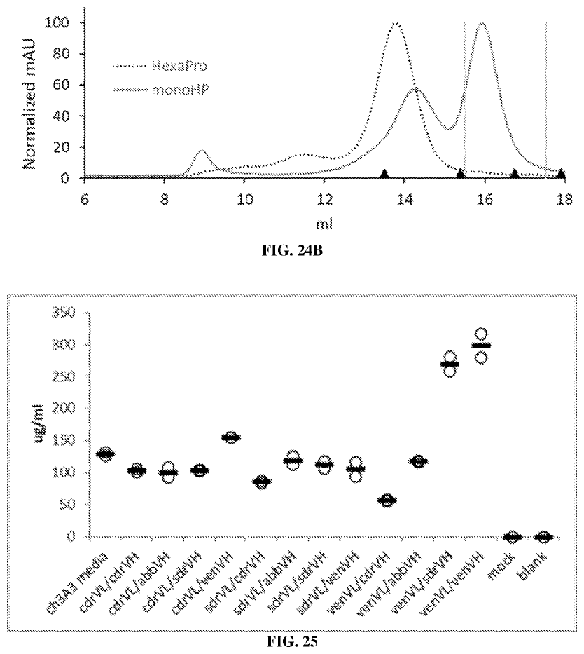

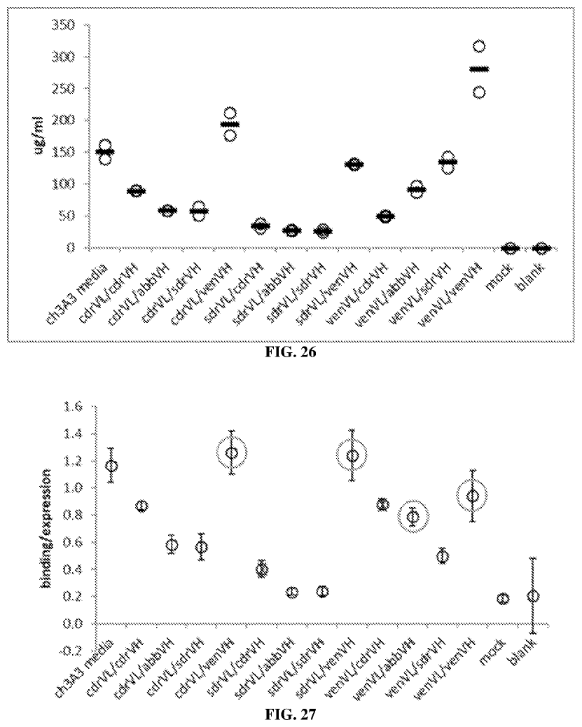

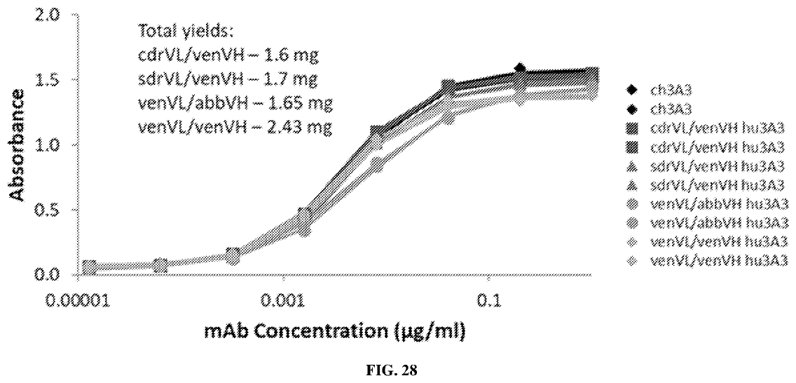

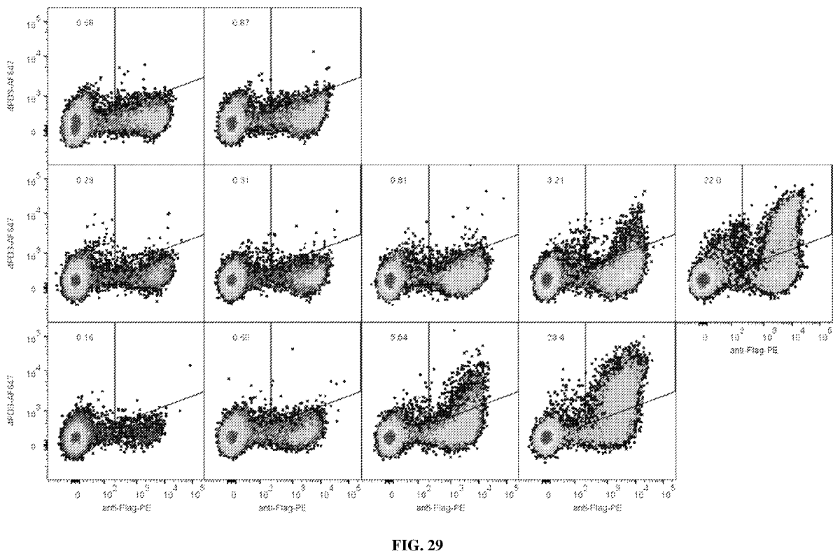

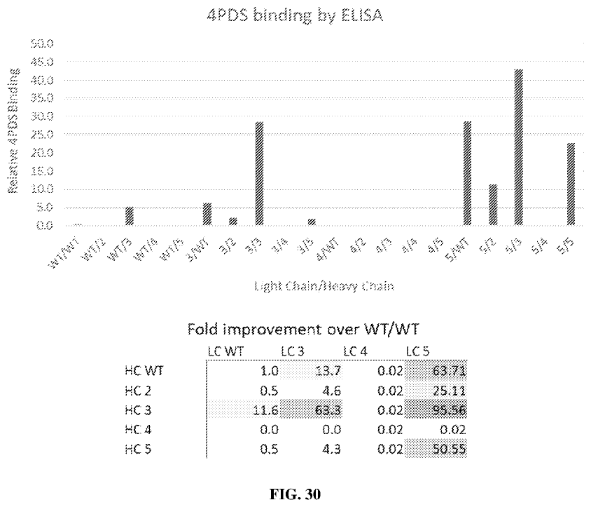

The ladder molecular weight is labeled in kDa on the left side. (FIG. 1C) ELISA capture of fresh (circles) or stressed (diamonds) SARS-2 spike on 3A3 coated plates. (FIG. 1D) Antibodies coated on ELISA plates captured fresh (left and right/middle, circles) or stressed (left/middle and right, diamonds) SARS-2 HP (left two) or SARS-2 (right two) spike proteins. For both (FIG. 1A) and (FIG. 1D), duplicate dilutions of spike over ˜5 log in concentration were used to calculate EC₅₀ values. For dilution series in which no binding was observed, EC₅₀ was assumed to be >1000 nM. Open symbols are replicate data and filled rectangles are average data.

FIGS. 2A-2I. Antibody 3A3 inhibits cellular fusion induced by the interaction of SARS-CoV-2 spike with human ACE2. HEK 293 cells stably expressing human ACE2 (HEK-ACE2) were stained with Cell Trace Far Red and incubated with a CHO-based cell line transiently expressing wild-type SARS-CoV-2 spike and EGFP and preincubated with 3A3 antibody or isotype control. (FIG. 2A) The cultures were imaged after 24 hours of incubation for EGFP (green) or Cell Trace Far Red (red) and the level of colocalization (yellow) was evaluated. (FIG. 2B) CHO cells not expressing SARS-CoV-2 spike and (FIG. 2C) HEK 293 cells not expressing ACE2 exhibited minimal fusion. (FIG. 2D) When the cultures were preincubated with an irrelevant isotype control antibody, extensive fusion and syncytia formation equivalent to no antibody was apparent. Incubation at (FIG. 2E) 1 μg/ml (6.7 nM), (FIG. 2F) 10 μg/ml (67 nM), and (FIG. 2G) 100 μg/ml (670 nM) 3A3 reduced fusion in a dose-dependent manner with significance reached at 10 μg/ml. (FIG. 2H) The percentage of HEK-ACE2 pixels (red) colocalizing with spike expressing CHO pixels (green) was analyzed with the JACoP plugin for ImageJ. Shown are the mean and standard deviation of at least 160 cells per condition from 8-9 independent images. The statistical analysis of colocalization percentages under different conditions were performed with ANOVA. (FIG. 2I) The same images per condition used in (FIG. 2H) were analyzed the average cell size of fused HEK-ACE2 with ImageJ as a second statistic method to test the cell fusion level. Shown are the mean and standard deviation of at least 160 cells per condition from 8-9 independent images. The statistical analysis of average cell sizes under different conditions were performed with ANOVA followed by Tukey's HSD test. Results shown are representative of four independent experiments; **** p<0.0001. Scale bar, 100 μm.

FIGS. 3A-3B. Infection with SARS-2 spike pseudotyped virus is inhibited by 3A3. (FIG. 3A) Lentivirus pseudotyped with wild-type (WT) or D614G SARS-2 spike protein or VSVG as a control were incubated with a range of 3A3 antibody concentrations and allowed to infect HEK293 cells expressing ACE2 receptor. 3A3 inhibited viral entry of the virus coated with either SARS-2 spike, with ˜10-fold greater potency for the D614G variant. IC₅₀ SARS-2 WT: 25.57 μg/ml; IC₅₀ SARS-2 D614G: 2.05 μg/ml. (FIG. 3B) Neutralization assays were performed with HIV particles pseudotyped with SARS-CoV-2 wild-type spike (SARS-2 spike, filled circles), the SARS-CoV-2 D614G mutant (D614G spike, triangles), B.1.1.7 SARS-CoV-2 (variant alpha) spike (B.1.1.7 spike, diamonds) and SARS-CoV spike (SARS-1 spike, squares). The pseudoviruses were incubated with different doses of each antibody (3A3, black; S309, blue; or an isotype control with irrelevant binding, grey) for 1 hour at room temperature before adding to HEK 293T cells stably expressing ACE2. Viral entry was detected by luciferase luminescence 60-72 hrs later. The entry efficiency of SARS-CoV-2 pseudoviruses without any treatment was considered 100%. Treatment of every pseudovirus with S309 or 3A3 resulted in significant (p<0.01) neutralization versus isotype control antibody with matched virus. Statistical analyses of neutralization IC₅₀ were performed with ANOVA followed by Tukey's HSD test.

FIGS. 4A-D. The 3A3 epitope is located at the apex of the S2 domain. (FIGS. 4A and 4B) Volcano plots showing changes in deuterium uptake in SARS-2 HexaPro spike peptides upon addition of 3A3 IgG (FIG. 4A) or Fab (FIG. 4B) after 10² s exchange. Significance cutoffs are an average change in deuterium uptake greater than 0.2 Da and a p-value less than 10⁻² in a Welch's t-test (hatched box). Black dots indicate peptides with a significant decrease in deuterium uptake and their boundaries are labeled. (FIG. 4C) Binding of 3A3 IgG and 3A3 Fab to 12 peptides that redundantly span residues 980 to 1006 of the SARS-2 HexaPro spike. Deuterium uptake plots for peptides with a significant decrease in deuterium uptake upon addition of 3A3 (see FIG. 17). Traces are SARS-2 HexaPro spike alone (black), with 3A3 IgG (blue), and with 3A3 Fab (orange). Error bars are ±²a from 3 or 4 technical replicates. Y-axis is 70% of max deuterium uptake assuming the N-terminal residue undergoes complete back-exchange. Data have not been corrected for back-exchange. (FIG. 4D) Mapping exchange difference on the S2 domain (PyMOL). Monomeric SARS-2 2P spike (PDB: 6VSB chain B) colored according to the difference in deuterium fractional uptake between SARS-2 HexaPro spike alone and with 3A3 IgG. The figure was prepared using DynamX per residue output without statistics and Pymol. Residues lacking coverage are indicated in grey. Structural features are labeled, including the 2P mutations at residues 986 and 987 (shown as sticks).

FIG. 5. Structural location of 3A3 epitope and implications for antibody binding. The 3A3 epitope (blue) within S2 is completely hidden by S1 in the structure of wild-type spike SARS-2 spike in the three RBDs down or closed conformation, PDB: 6XR8. In structures of stabilized spike with one RBD up (PDB: 6VSB), two RBDs up (PDB: 7A93), or three RBDs up (PDB: 7A98) and bound to ACE2 (purple) (PDB: 7A98), the 3A3 epitope is increasingly accessible. White indicates areas covered by HDX-MS, while no peptides were recovered in regions in grey.

FIGS. 6A-6I. The 3A3 epitope is inaccessible in the closed conformation of the SARS-2 spike. (FIG. 6A) By BLI, the control antibody 2-4 (Liu et al., 2020) bound both SARS-CoV-2 HexaPro (solid) and HexaPro locked into the “closed” conformation (dashed). 3A3 was able to capture HexaPro (solid), but not “closed” HexaPro (dashed). Vertical dashed lines indicate start of dissociation phase. The 3A3 epitope identified by HDX mass spectrometry (SARS-CoV-2 amino acids 980-1006) is highly conserved across the spike (FIG. 6B) sequences and (FIG. 6D) structures of coronaviruses known to infect humans. In (FIG. 6B), identical residues are highlighted in yellow and similar residues are highlighted in aqua. Solvent exposed residues visible in spike structures with at least one RBD up are underlined in the SARS-CoV-2 sequence. The location of the two proline mutations introduced to 2P variants are shown below the alignment. In (FIG. 6C), identical residues are indicated by a dot and similar residues are highlighted in grey. Residues conserved across all listed β-coronaviruses are in bold in the SARS-CoV-2 epitope. The location of the two proline mutations introduced to 2P variants are shown below the alignment. In (FIG. 6D), the structure of each epitope is displayed as follows: SARS-2 (6VSB)—red, SARS-1 (6CRV, RMSD=1.2 Å)—orange, MERS (5X5C, RMSD=3.5 Å)—blue, NL63 (7KIP, RMSD=2.6 Å)—grey, HKU1 (5I08, RMSD=0.8 Å)—teal, OC43 (60HW, RMSD=0.7 Å)—green, 229E (6U7H, RMSD=2.0 Å)—yellow. In (FIG. 6E), the structure of each epitope is displayed as follows: SARS-2 (6VSB)—red, SARS-1 (6CRV, RMSD=0.8 Å)—magenta, MERS-CoV (5X5C, RMSD=3.1 Å)—blue, HKU1 (5I08, RMSD=0.5 Å)—teal, OC43 (6OHW, RMSD=0.6 Å)—green. (FIG. 6F) The 3A3 epitope is accessible on unmodified SARS-CoV-2 spike. Antibody 3A3 weakly stains wild-type SARS-CoV-2 spike (WT) expressing Expi293 cells, and more strongly binds SARS-CoV-2 D614G spike expressing cells in flow cytometry. Control Expi293 cells not expressing spike (mock) are shown in grey. (FIG. 6G) Single point mutants of HexaPro had increased or decreased binding to 3A3 relative to HexaPro, indicating residues important for 3A3 binding. Each mutant was tested in duplicate in two to five independent ELISA assays. Significance of the difference relative to unmutated HexaPro was determined by ANOVA with post-hoc Tukey-Kramer test with α=0.05 (*) and α=0.01 (**). (FIG. 6H) Location of the mutations that altered binding to 3A3 in the HexaPro spike (6XKL) monomer (left) and (FIG. 6I) in the context of full spike (top down of the S2 portion of the trimer, top; side view of the full spike trimer, bottom). All epitope residues (980-1006) are shown in space-fill, with those not mutated in grey and those mutated without impact on 3A3 binding in black. Locations of mutations that improved binding are displayed in orange and mutations that reduced binding are shown in teal.

FIGS. 7A-7B. The 3A3 epitope is accessible only when the spike is open. (FIG. 7A) Antibody 3A3 or S309 was coupled to anti-Fc BLI sensors and allowed to bind monomeric HexaPro (monoHP), E1031R HexaPro (E1031R), HexaPro, or SARS-CoV-2 S2P (SARS-2) spike protein at several concentrations. The proteins were freshly thawed from −80° C. (fresh), incubated at 4° C. for one day for SARS-2 or 1 week for HexaPro (4° C.), or incubated at 37° C. for 1 day (37° C.). The on-rate of binding (kₒₙ) was calculated from measurements at seven concentrations in two independent experiments and error is the range of these measurements. (FIG. 7B) Antibody 3A3 or 2-4 were coupled to anti-Fc BLI sensors and allowed to capture HexaPro or nothing (buffer), then dipped into buffer (baseline), and finally dipped into ACE2-Fc (ACE2) or nothing (buffer).

FIGS. 8A-8D. Sequence conservation is higher in the S2 domain than the S1 domain across coronaviruses that infect humans. Percent sequence identity and similarity between the spike (FIGS. 8A and 8C, respectively) S1 subunits and (FIGS. 8B and 8D, respectively) S2 subunits of the seven coronaviruses known to infect humans was analyzed using LALIGN/PLALIGN local alignment (available at fastademo.bioch.virginia.edu/fasta_www2/fasta_www.cgi?rm=lplalign). The default values of the gap penalties of open=−12 and gap=−2 were used for S2 alignments, but reduced to open=−10 and gap=−1 to allow for variable domain lengths in S1. GenBank protein sequence identification numbers (protein_id) used in these alignments were: NP_073551.1 for 229E, AFD64754.1 for NL63, BBA20979.1 for OC43, BBA20986.1 for HKU1, YP_009047204.1 for MERS, AYV99817.1 for SARS-CoV-1, and YP_009724390.1 for SARS-CoV-2.

FIG. 9. Many cross-reactive scFv-phage target the foldon domain. The majority of monoclonal phage tested by ELISA on SARS-CoV-2 spike or the unrelated RSV F foldon coated plates had cross-reactive binding, indicating targeting of the shared foldon domain. These data, after round 4 of panning, show 3A3 in black, a close relative of 3A3 (two amino acid changes) is the gray diamond within the cluster of black diamonds, 4A5 is the diamond above the black diamonds at about 0.35 absorbance, 4H2 is the top left diamond, and the rest being foldon binders with closely related CDRH3 sequences.

FIGS. 10A-10C. The purified 3A3, 4A5, 4H2, and 3E11 full-length antibodies are pure and stable. (FIG. 10A) SDS-PAGE was used to evaluate 3 μg of each antibody in either reduced (R) or non-reduced (NR) states. Molecular weights of each ladder band in kDa are indicated on the left. (FIG. 10B) 100 μg of each antibody was evaluated by analytical SEC on an S200 column. Standards (marked with an x) are: thyroglobulin (669 kDa, 8.609 ml); ferritin (440 kDa, 10.316 ml); beta amylase (200 kDa, 11.743 ml); aldolase (158 kDa, 12.574 ml); conalbumin (75 kDa, 14.131 ml); ovalbumin (44 kDa, 15.079 ml); carbonic anhydrase (29 kDa, 16.484 ml); cytochrome c (12.4 kDa, 18.046 ml). The right most curve is 4A5. (FIG. 10C) Thermal unfolding of 3A3, 4A5, 4H2, and 3E11 (right most curve) show profiles typical of antibodies. Thermal melt temperatures corresponding to the second peak minimum are reported in Tables 9 and 10.

FIGS. 11A-11D. SPR and BLI analysis of 3A3, 4A5, 4H2 and 3E11 mAbs measured low to mid nanomolar affinity (K_{d}) for binding to SARS-CoV-2 spike variants. Binding of 3A3 Fab to HexaPro S2 measured by SPR (FIG. 11A) analysis and BLI (FIG. 11B). Equilibrium affinity of immobilized full-length IgGs on anti-Fc sensors capturing the indicated (FIG. 11C) SARS-CoV-2 spike or S2 domain and (FIG. 11D) SARS-CoV or MERS-CoV spike. Measurements were performed by dipping full-length antibody-coated sensors into serial dilutions of the different spike variants (100 to 1.56 nM) followed by a dissociation step in the buffer. Vertical lines indicate the start of the dissociation phase; data shown in solid lines with fits shown in dashed lines.

FIG. 12. Antibodies 3A3, 4A5, and 4H2 bind highly pathogenic coronaviruses. SARS-CoV-2 spike (SARS-2), SARS-CoV-2 HexaPro spike (SARS-2 HP), SARS-CoV spike (SARS-1), MERS-CoV spike, and HKU1 spike, RSV F, and milk (no coat) were coated on high binding plates for ELISA. Full length antibodies were serially diluted in duplicate and allowed to bind the coat proteins, then binding was detected with anti-human Fc-HRP development of TMB substrate. The binding data and antibody concentrations for each replicate were fit to a four-parameter logistic curve. Inverse EC₅₀ of these data are presented in FIG. 1A. This experiment was repeated twice; data shown are representative of replicate experiments.

FIG. 13. SDS-PAGE analysis of fresh and stressed SARS-CoV-2 and SARS-CoV-2 HexaPro spikes shows substantial aggregation of the stressed SARS-CoV-2 spike. For each spike, 8 μg of protein was analyzed by SDS-PAGE under non-reducing conditions. Reduced band intensity of stressed SARS-CoV-2 spike (bottom arrow) and appearance of a band just under the loading wells (top arrow) indicates aggregation products not apparent in the other samples. Densitometry analysis (ImageJ) of these bands indicates that the main peak in the stressed SARS-CoV-2 spike is diminished by ˜20% relative to the fresh SARS-CoV-2 spike intensity. Ladder molecular weights in kDa are indicated to the left of the gel image.

FIG. 14A-14B. Antibodies 4A5, 4H2, and 3E11 have no impact on cellular fusion. HEK 293 cells stably expressing ACE2 were stained with Cell Trace Far Red and mixed with CHO cells expressing only EGFP (GFP only) or CHO cells expressing EGFP and wild-type SARS-CoV-2 spike and preincubated with 30 μg/ml (200 nM) of: no antibody, isotype control, 3A3, 4H2, 4A5, or 3E11. After evaluating (FIG. 14A) the percentage of cells with colocalized red and green fluorescence and (FIG. 14B) the average HEK-ACE2 cell size, only incubation with 3A3 was found to significantly reduce fusion. Shown are the mean and standard deviation of at least 100 cells per condition from 5-7 independent images. The statistical analysis was performed with ANOVA. Results show are representative of three independent experiments; * p<0.05, **** p<0.0001.

FIG. 15. Peptides monitored through all timepoints of deuteration for SARS-CoV-2 HexaPro spike alone and with 3A3 IgG or Fab. A total of 192 peptides were monitored, covering 56.3% of the SARS-CoV-2 HexaPro spike sequence and averaging 3.34 redundancy per amino acid. All peptides were manually checked. SARS-CoV-2 spike features are indicated on the sequence, including glycosylation sites. Glycosylation was not included in the peptide search and so no peptides are recovered surrounding these sites. The sequence corresponds to SEQ ID NO: 67. The amino acids at positions 682-685, 986, 987, and 1195-1288 are altered from the SARS-CoV-2 wildtype spike sequence.

FIG. 16. Deuterium uptake of SARS-CoV-2 HexaPro spike alone suggests the trimer is maintained under HDX conditions. Trimeric (i and ii) and monomeric (iii) SARS-CoV-2 2P spike (PDB: 6VSB) colored according to fractional deuterium uptake of the SARS-CoV-2 HexaPro spike alone after 10³ s of exchange. The figure was prepared using DynamX per residue output and Pymol. Residues lacking coverage are indicated in grey. Structural features are labeled, including the central trimer interface that shows relatively low deuterium incorporation.

FIGS. 17A-B. Location of the 3A3 epitope in SARS-CoV-2 spike. (FIG. 17A) Additional deuterium uptake plots for peptides with a significant decrease in deuterium uptake upon addition of 3A3 (see also FIG. 6G). Traces are SARS-CoV-2 HexaPro spike alone (black), with 3A3 IgG (blue), and with 3A3 Fab (orange). Error bars are ±2σ from 3 or 4 technical replicates. Y-axis is 70% of max deuterium uptake assuming the N-terminal residue undergoes complete back-exchange. Data have not been corrected for back-exchange. (FIG. 17B) Monomeric SARS-CoV-2 2P spike (PDB: 6VSB chain B) colored according to the difference in deuterium fractional uptake between SARS-CoV-2 HexaPro spike alone and with 3A3 Fab. The figure was prepared using DynamX per residue output without statistics and Pymol. Residues lacking coverage are indicated in grey. Structural features are labeled, including the 2P mutations at residues 986 and 987 (shown as sticks).

FIGS. 18A-18C. 4A5 and 4H2 do not bind the same epitope as 3A3. Volcano plots showing changes in deuterium uptake in SARS-CoV-2 HexaPro spike peptides upon addition of 4A5 (FIG. 18A) or 4H2 (FIG. 18B) after 10² s exchange. Significance cutoffs are an average change in deuterium uptake greater than 0.2 Da and a p-value less than 10⁻² in a Welch's t-test (hatched box). (FIG. 18C) Deuterium uptake plots for peptides from the 3A3 epitope. Traces are SARS-CoV-2 HexaPro spike alone (black), with 4A5 (blue), and with 4H2 (orange). Error bars are ±²a from 4 technical replicates. Y-axis is 70% of max deuterium uptake assuming the N-terminal residue undergoes complete back-exchange. Data have not been corrected for back-exchange-all traces overlay.

FIG. 19. Antibody 3A3 cannot bind the MERS S2 domain with the apex removed. To evaluate 3A3 binding to MERS S2 with the apex region containing the 3A3 epitope removed by BLI, anti-human Fc Sensors were used to pick up 3A3 (10 nM) or an S2 binding control antibody IgG22 to a response of 0.6 nm. Then mAb coated tips were dipped into wells containing MERS-S2 or apex-less MERS-S2 (100 nM). Data were collected over an antibody association to the MERS S2 domains for 5 minutes and dissociation for 5 minutes. The solid line shows data collected; the black dashed line shows the best fit. The lines, from top to bottom at the 600 s time point, represent: IgG22 to MERS-S2, IgG22 to Apex-less MERS-S2, IgG3A3 to MERS-S2, and IgG3A3 to Apex-less MERS-S2.

FIG. 20. Spike can simultaneously bind 3A3 and 5309 (an RBD binder) by BLI. To evaluate 3A3 binding to HexaPro captured by an RBD binder (IgGS309), Anti-Human Fc Sensors were used to pick up IgGS309 (20 nM) to a response of 0.6 nm. Then mAb coated tips were dipped into wells containing HexaPro (50 nM) to a response of 0.6 nm and then dipped into wells containing murine-3A3 (100 nM), irrelevant murine mAb (100 nM), or buffer. Association of m3A3/irrelevant mAb was measured for 5 min and dissociation for 10 min.

FIG. 21. HexaPro variants with reduced 3A3 binding retain trimer SEC profile. The single point mutants of HexaPro, D985L, E9881, D994A, and L1001A, had reduced binding to 3A3 relative to HexaPro (FIG. 4C), but retained the overall size of unmodified HexaPro by SEC with elution of the main peak at ˜13.75 mL on a Superose 6 Increase 30/100 column, indicating the spike was intact. Molecular weight markers (black triangles) on the x-axis are peak elution volumes from the following standards in order from left to right: thyroglobulin (669 kDa, 13.49 mL), ferritin (440 kDa, 15.40 mL), β-amylase (200 kDa, 16.75 mL), aldolase (158 kDa, 17.91 mL).

FIGS. 22A-22B. Binding of 3A3 to spike is impacted by accessibility to the epitope. (FIG. 22A) Binding of 3A3 and S309 to SARS-CoV-2 spikes were evaluated by BLI. Full length antibodies were captured by anti-human Fc sensors, dipped into serial dilutions of spike from 25 to 0.78 nM and then incubated in buffer. SARS-CoV-2 S2P or HexaPro spike was freshly thawed from preparations immediately stored at −80° C. (fresh), stored at 4° C. for 1-3 weeks (4° C.), or stored at 37° C. for 24 hours (37° C.) before dilution into buffer immediately before BLI analysis. Vertical lines indicate the start of the dissociation phase. Off-rates with a trimeric analyte (HexaPro, SARS-CoV-2, and E1031R spikes) are not reliable due to rebinding. (FIG. 22B) Equilibrium K_{d} and on-rates were calculated based on seven concentrations in two independent experiments (representative data shown in FIG. 22A). For all fits the coefficient of determination (R²) was >0.98 and Rₘₐₓ was between 0.7 and 1.4 nM.

FIG. 23. Epitope accessibility affects antibody binding by ELISA. SARS-CoV-2 S2P or HexaPro spike stored at 4° C. for 1-3 weeks (4° C., top curves), or stored at 37° C. for 24 hours (37° C., bottom curves) were allowed to bind to 3A3, 5309, or 3E11 coated plates for one hour at room temperature. Detection with StrepTactin-HRP and TMB substrate showed reduced binding of 3A3 after incubation at 37° C. Control antibodies S309 and 3E11 had somewhat reduced binding to SARS-CoV-2 spike incubated at 37° C. likely due to protein degradation, aggregation, or tag loss.

FIGS. 24A-24B. HexaPro variants expose the 3A3 epitope. (FIG. 24A) The kinetics of interconversion between the closed-S2 and open-S2 states of HexaPro (top lines) and HexaPro E1031R (bottom lines) were evaluated by HDX-MS as previously described (Costello, Shoemaker, et al 2021). The spike proteins were incubated at 37° C. for 24 hours, then incubated at 4° C., assayed for detection of the S2-open state over time, then transferred to 37° C. and assayed again. The fraction closed-S2 was estimated by exposing a sample of the incubation reaction to a 1 min pulse of deuterium and quenching the labeling reaction with low pH and temperature. After LC-MS and peptide identification, the bimodal mass envelopes for all timepoints for one peptide were globally fit to a sum of two gaussians, keeping the center and width of each gaussian constant across all incubation time points. After fitting, the area under the lower molecular weight gaussian was integrated to determine the fraction of closed-S2. For HexaPro, the half-life of conversion from closed to open (37° C.→4° C. was 143.5 h and the half-life of conversion from open to closed (4° C.→37° C.) was 2.5 h. These conversions for HexaPro E1031R (bottom lines) occur with half-lives decreased by >700-fold (0.2 h) and >6-fold (0.4 h), respectively. (FIG. 24B) An HRV3C cut site was inserted between the HexaPro spike c-terminus and the foldon domain allowing expression of the spike as a trimer, incubation at 4° C. for 5 days, then digestion with HRV3C protease to produce HexaPro monomer (monoHP). After digestion, the sample was purified by SEC on a Superose6 column to isolate monoHP (blue line) from partially digested products. Fractions between the yellow lines were collected for use in BLI. Molecular weight markers (black triangles) on the x-axis are peak elution volumes from the following standards in order from left to right: thyroglobulin (669 kDa, 13.49 mL), ferritin (440 kDa, 15.40 mL), β-amylase (200 kDa, 16.75 mL), aldolase (158 kDa, 17.91 mL).

FIG. 25. Comparison of the expression level of antibody in the media of ExpiCHO cells. ELISA plates were coated with anti-human-Fc antibodies, then human antibodies in dilutions of the media were captured on the plate and detected with anti-human-kappa-HRP.

FIG. 26. Comparison of HexaPro binding of antibody in the media of ExpiCHO cells. ELISA plates were coated with HexaPro SARS-CoV-2 spike, then human antibodies in dilutions of the media were captured on the plate and detected with anti-human-Fc-HRP.

FIG. 27. Comparison of normalized HexaPro binding of antibody in the media of ExpiCHO cells. Taking a ratio of the data from FIG. 2 and FIG. 1 allows us to compare the binding of each antibody independent of expression level. Some antibodies (red X) had such low expression (FIG. 1) that they were eliminated for consideration. Others had promising media ELISA profiles and were advanced to evaluation of purified antibody (blue circles).

FIG. 28. Comparison of HexaPro binding of purified antibodies. Dilutions and yields of purified antibodies were compared by ELISA with HexaPro SARS-CoV-2 spike coated on plates and detection with anti-human-Fc-HRP.

FIG. 29. Affinity maturation library sorting. The site directed library was sorted for binding to 4PDS at 50 nM by magnetic cell sorting (R1), and three rounds of flow cytometric sorting (R2-R4). The error prone library was sorted with three rounds of flow cytometric cell sorting (R1-R3). For flow cytometry, the yeast display vector included a peptide Flag tag on the light chain to allow staining for expression and proper Fab assembly with anti-Flag-PE. The 4PDS spike was directly labeled with Alexa Fluor 647.

FIG. 30. Combinatorial of affinity matured antibodies. SARS-CoV-2 4P or 4PDS spike (50 nM, 10 nM, 2 nM) were allowed to bind to 3A3 variant coated plates. Detection was with StrepTactin-HRP.

### DETAILED DESCRIPTION

Three strains of highly pathogenic human coronaviruses have emerged over the last 20 years, with SARS-CoV-2 causing a global pandemic. While vaccines and therapeutic antibodies targeting the SARS-CoV-2 spike appear highly effective, many of these approaches focus on the poorly conserved receptor-binding domain (RBD). Strategies to protect against future coronaviruses will depend on identifying essential neutralizing epitopes, which may be present in the more conserved S2 domain. Provided herein are three antibodies that bind unique epitopes conserved on the pre-fusion core of MERS-CoV, SARS-CoV, and SARS-CoV-2 spike S2 domains. One of these, antibody 3A3 binds a conformational epitope with ˜2.5 nM affinity and neutralizes in in vitro SARS-CoV-2 cell fusion and pseudovirus assays. Hydrogen-deuterium exchange mass spectrophotometry identified residues 980-1006 in the flexible hinge region at the S2 apex as the 3A3 epitope. Binding of 3A3 to natural and engineered spike variants increases with RBD-up propensity, consistent with increased accessibility of the identified epitope. Antibody 3A3 binding may span several protomers, which could prevent the global S2 conformational rearrangements required for virus-host cell fusion by preventing hinge straightening and protomer re-positioning. This work defines the first highly conserved vulnerable site on the SARS-CoV-2 S2 domain, providing insight into the constraints imposed by spike function on antigenic variation and may contribute to design of pan-protective spike immunogens.

### I. Aspects of the Present Invention

The regular emergence of pathogenic coronaviruses over the past two decades motivated efforts to isolate cross-reactive coronavirus spike antibodies. In all highly pathogenic coronaviruses, the spike protein is responsible for targeting host cells via the S1 domain, which has little sequence similarity across coronaviruses (41-87%; FIGS. 8A-8D). In contrast, the S2 domain mediates viral envelope and target cell membrane fusion through a complex conformational change (Ng et al., 2020; Cai et al., 2020), with a correspondingly low tolerance for sequence variation (63-98% similarity; FIGS. 8A-8D). Moreover, neutralizing sera from individuals never exposed to SARS-2 is common in young people and exclusively bind the S2 domain (Ng et al., 2020). Accordingly, S2 is an attractive target for pan-coronavirus antibody therapy and vaccination. With this in mind, three highly cross-reactive antibodies that bind S2 were isolated from a MERS S2 immune phage library and characterized.

All three antibodies bind SARS-CoV-2 HexaPro with low- to mid-nanomolar K_{d} values (Tables 9 and 10) and exhibit similar binding to SARS-CoV-2 HexaPro, SARS-CoV, SARS-CoV-2, and MERS-CoV spikes in ELISA assays, with minimal binding to HKU1 spike (FIG. 1A). Antibodies 4H2 and 4A5 bind conserved linear epitopes but do not neutralize in cell fusion or pseudovirus assays. By contrast, antibody 3A3 binds a conformational epitope and exhibits selectivity for fresh versus stressed SARS-CoV-2 spike (EC₅₀˜125-fold higher), suggesting 3A3 could be used to monitor spike quality (FIGS. 1C, 1D). Moreover, 3A3 binds SARS-CoV-2 spike when expressed on the mammalian cell surface and is neutralizing in multiple in vitro assays (FIGS. 2, 3), with greater sensitivity for the more transmissible SARS-CoV-2 D614G variant.

Antibody 3A3 binds a highly conserved conformational epitope near the S2 hinge and binds the isolated SARS-CoV-2 HexaPro S2 domain with a low-nanomolar K_{d} by SPR (Tables 9 and 10, FIG. 11). It exhibits similar binding to the full ectodomain of SARS-CoV-2 HexaPro, SARS-CoV, SARS-CoV-2, and MERS-CoV spikes in ELISA and BLI assays (K_{d} values between 2.5 and 23 nM; FIG. 1, Tables 9 and 10, and FIGS. 11 and 12), with minimal binding to the less pathogenic HKU1 spike (FIG. 1A). In addition, 3A3 neutralizes SARS-CoV and SARS-CoV-2 spikes in multiple in vitro assays (FIG. 3), with greater potency for the more transmissible D614G and B.1.1.7 variants. The 3A3 binding site was identified by HDX-MS as cryptic epitope at the apex of S2 (residues 986-1006; FIG. 4). This epitope is conformational, as 3A3 is unable to bind spike stressed by heat and freeze-thaw treatments (FIG. 1). Mutational analysis identified two hot spot residues (D985 and E988) which flank the 2P stabilizing mutations at the “jackknife hinge” between HR1 and the central helix (FIGS. 6E-6I). This hairpin converts into a long, straight helix during viral fusion, suggesting that 3A3 neutralizes by preventing hairpin extension.

Recent work revealed that 3A3 can only bind SARS-CoV-2 spike when S2 opens to expose the trimer interfaces (Costello et al., 2021), suggesting this state is required for 3A3 protection in neutralization assays. Analysis of spike pre-fusion structures shows the 3A3 epitope to be completely obscured when the RBDs are in the three-down or “closed” conformation but is partially exposed when the RBDs become uncoupled from neighboring NTDs (Wrapp et al., 2020; Hsieh et al., 2020) and fully exposed when S2 opens (Costello et al., 2021). Our biochemical data show that 3A3 does not bind spike locked in the closed, three RBD “down” state (FIG. 6A) but more rapidly associates with multiple SARS-CoV-2 HexaPro variants having exposed S2 interfaces or when spike is treated to favor the S2-open state (FIG. 7A). D614G and B.1.1.7 pseudotyped virus are more readily neutralized by 3A3 than SARS-CoV-2 spike pseudovirus, indicating that the S2-open versus S2-closed ratio and epitope accessibility are potentially altered in these more infectious variants (Plante et al., 2021; Davies et al., 2021) (FIG. 3). Conversely, 3A3 only weakly binds the HKU1 spike, which contains three non-conservative changes within the 3A3 epitope (FIG. 6C). None of these seem critical for 3A3 binding (FIG. 6G), suggesting the loss of binding is due to reduced epitope accessibility. For SARS-CoV-2 HexaPro spike, ACE2 binding biases the spike towards the S2-open state (Costello et al., 2021), allowing 3A3 to bind the ACE2-spike complex (FIG. 7B), and suggesting that ACE2 engagement may expose the 3A3 epitope in all variants.

The 3A3 epitope is highly conserved across human coronaviruses. This epitope has high sequence identity (45%) and similarity (65%, FIG. 4C) among seven human coronaviruses. Moreover, 3A3's ability to bind SARS-CoV, SARS-CoV-2, and MERS-CoV spikes with similar sensitivities supports the concept that while binding depends on RBD position, it is independent of RBD sequence or receptor specificity. The structure of the 3A3 epitope is highly conserved across wild-type (PBD 6XR8), 2P (PDB 6VSB), HexaPro (PDB 6XKL), stabilized D614G (PDB 6XS6), and non-stabilized D614G (PDB 7KDL) SARS-CoV-2 spike with RMSD of ≤0.8 Å, but efficiency of 3A3 binding is altered for each, indicating that epitope accessibility impacts 3A3 binding affinity. Binding to HKU1 spike may be weakly detected by ELISA because these spikes rarely sample the RBD up configuration, even in stabilized forms, and the 3A3 epitope is poorly accessible (Walls et al., 2020).

Despite success in identifying neutralizing S1 epitopes on SARS-CoV-2 that block ACE2 interactions (Rappazzo et al., 2021; Goike et al., 2021; Wu et al., 2020; Chen et al., 2020; Jones et al., 2021; Wrapp et al., 2020; Barnes et al., 2020), even broadly neutralizing RBD antibodies may be susceptible to escape (Greaney et al., 2021; Starr et al., 2021; Andreano et al., 2020) which has motivated interest in the more conserved S2 domain. Over 200 S2 targeting antibodies have been reported (Raybould et al., 2021), but very few have been described in detail. Chi et al. described over twenty SARS-CoV-2 S2 antibodies, several of which are neutralizing but do not discuss their cross-reactive binding, epitopes, or mechanisms of neutralization (Chi et al., 2020). In other work, anti-SARS-CoV-2 S2 antibodies were reported that cross-react with SARS-CoV, MERS-CoV, HKU1 and/or OC43 spike proteins (Song et al., 2021). One antibody binding SARS-CoV, SARS-CoV-2 and MERS-CoV spike neutralizes SARS-CoV and SARS-CoV-2 pseudovirus by recognizing an epitope near the S2 base, opposite the 3A3 apex epitope. Recently SARS-CoV, SARS-CoV-2 and MERS-CoV neutralizing antibodies were reported that bind in this same general region (Pinto et al., 2021; Sauer et al., 2021). By contrast, antibody 3A3, which was elicited by mouse immunization with MERS-CoV S2 and selected with SARS-CoV-2 spike, is highly cross-reactive with the MERS-CoV and SARS-CoV spikes and identifies a novel neutralizing epitope. Interestingly, elicitation of antibodies binding this epitope is not limited to immunization with designed immunogens. An antibody repertoire profiling effort recovered Ab127 from a convalescent COVID-19 patient (Jones et al., 2021) and used HDX to determine that it protects peptides 980-1006 (overlapping the 3A3 epitope) and 1179-1186 and binds isolated S2 with a 471 nM affinity. While Ab127 did not neutralize, likely due to its poor affinity, this suggests that the 3A3 epitope is immunogenic during infection.

Broadly neutralizing antibodies binding epitopes on the fusogen trimer interface, similar to 3A3, have recently been reported for other viruses. Conformations analogous to the S2-open state were described for the RSV F (Gilman et al., 2019) and VSV G (Kim et al., 2017) fusogens, suggesting many fusogenic proteins open during conformational change to expose interfacial epitopes. In a few cases, antibodies binding epitopes exposed only in this open state have been described. Antibody D5 and derivatives bind a highly conserved epitope on the N-heptad repeat region of the HIV-1 prehairpin-intermediate to neutralize a wide range of strains (Montefiori et al., 2021; Rubio et al., 2021). Similarly, the broadly neutralizing human antibody A20 recognizes a site at the influenza hemagglutinin head trimer interface to disrupt the trimer (Bangaru et al., 2019). Lee et al identified protective anti-flu antibodies that bind different epitopes present on monomeric but not trimeric hemagglutinin (Lee et al., 2016). Collectively, these data underscore the novelty of the 3A3 epitope and highlight conserved interfacial epitopes as an emerging strategy for development of broadly neutralizing anti-viral antibodies. As we learn more about the 3A3 paratope, it may serve as a starting point for design of antibodies recognizing even more diverse coronavirus spikes at the structurally conserved S2 hinge. Higher affinity (sub-nanomolar) binding to the spike hinge may overcome accessibility limitations in spikes variants that do not favor the S2-open state.

The location of the 3A3 epitope suggests a spike neutralizing mechanism. The 3A3 epitope is near the “jackknife hinge” on the S2 domain between HR1 and the central helix, which converts from a hairpin turn into a long, straight helix. This transition is the key event in spike conformational change; accordingly, the stabilizing “2P” proline substitutions are also located in this region and there are no glycosylation sites to impede the transition (Watanabe et al., 2020). Binding of 3A3 to the pre-fusion hairpin region near the trimer epicenter may lock spike in its pre-fusion conformation and thereby prevent movement of the fusion peptide. Antibody 3A3's extreme sensitivity to spike conformational changes and lack of recognition of the denatured linear peptide in SDS-PAGE suggest 3A3 binding may span several protomers.

Although S2 targeting antibodies exist, very few S2 specific antibodies have been reported, and even fewer have been described in detail. Chi et al. describe over twenty SARS-CoV-2 S2 antibodies, several of which are neutralizing, but do not discuss their cross-reactive binding, epitopes, or mechanisms of neutralization (Chi et al., 2020). In other work, an anti-S2 antibody was reported to bind the SARS-CoV-2 and HKU1 spikes near the S2 base, opposite the 3A3 apex epitope, with weakly neutralizing activity (Song et al., 2020), and recently a MERS/OC43 binding antibody was reported that binds this same region and neutralizes MERS (Sauer et al., 2020). Antibodies neutralizing SARS-CoV epitopes at the base of S2 on the HR2 or HR1 peptides were also reported but not fully characterized (Lip et al., 2006; Elshabrawy et al., 2012).

Therapeutic antibody cocktails may benefit from inclusion of neutralizing antibodies targeting S2, such as a humanized 3A3, since simultaneous targeting of multiple epitopes can improve protection and reduce the risk of escape variants. For example, antibodies binding the MERS S2 domain are potently neutralizing individually, but synergize with antibodies blocking receptor binding and prevent the emergence of viral escape mutants only when used together (Wang et al., 2018). However, SARS-CoV-2 cocktails currently approved or in development focus on the RBD and S1 (Renn et al., 2020). Regeneron's approved therapeutic cocktail REGN-COV2 is a mixture of two noncompeting RBD binding antibodies. SARS-CoV-2 spike variants have already been identified that can evade both antibodies, one requiring only a single amino acid substitution and another found in a patient after treatment with REGN-COV2 (Starr et al., 2020).

In addition to antibody evasion concerns, free S1 generated during infection or via spontaneous dissociation (Cai et al., 2020; Zhang et al., 2020) may bind antibodies targeting S1. Inclusion of neutralizing antibodies targeting S2 in therapeutic cocktails may provide a second layer of defense. Antibody 3A3, in particular, only binds spike in its active, fusion-capable form and cannot be depleted by post-fusion spike byproducts. Compared to antibodies that must compete with ACE2 for binding to the RBD-ACE2 interaction site, the 3A3 epitope is available without competition once exposed. Broadly neutralizing S2 antibodies could be stockpiled to protect healthcare workers against future novel coronavirus outbreaks until specific therapies are developed.

Mapping conserved, especially neutralizing, spike epitopes will help guide design of broadly protective immunogens able to protect against all highly pathogenic human coronaviruses. This will include spike engineering to reduce the immunodominance of some regions while increasing the immunogenicity of conserved epitopes. While S2 antibodies are elicited by infection, and can be both neutralizing and cross-reactive, the vast majority of naturally occurring neutralizing antibodies target the immunodominant RBD (Zost et al., 2020). The S2-binding antibodies described here were generated by direct immunization with stabilized S2 or spike engineered to stabilize the RBD's in the “up” position to make the 3A3 epitope more accessible, suggesting that immunization with a mixture of spike and isolated S2 may promote generation of anti-S2 antibodies.

A related concern is the immunogenicity of the foldon domain, as indicated by the high frequency of foldon-binding scFvs isolated in this work. The implication is that individuals immunized with an antigen containing foldon (e.g. RSV-F (Crank et al., 2019), currently in Phase 2 clinical trials) may preferentially expand a foldon-responsive B-cell population upon boosting or immunization with a second foldon-containing antigen. Existing anti-foldon antibodies may also rapidly clear a new foldon-containing antigen before a robust response is generated. If future studies support this hypothesis, fold-on immunogenicity could be reduced by strategies such as the introduction of glycosylation sites to shield unprotective immunogenic epitopes (Sliepen et al., 2015).

Spike engineering may be necessary to better present cryptic epitopes, such as that recognized by 3A3, to the immune system. It is unclear how common antibodies binding cryptic epitopes are, although the stabilized spikes used in several vaccines more readily sample the RBD up conformations thereby exposing the 3A3 and other cryptic epitopes, such as that recognized by the SARS-CoV/SARS-CoV-2 cross-reactive RBD binding antibody CR3022 (Yuan et al., 2020). Vaccines employing inactivated or attenuated virus may be less likely to expose these epitopes since most wild-type spike appears to exist in the closed conformation with RBDs that rarely sample the up conformation. To increase the accessibility of 3A3 and CR3022 epitopes, spike immunogens could be engineered to bias or lock the RBDs in the up position, although use of stabilized 2P or HexaPro or D614G SARS-CoV-2 spikes already stabilize the RBD-up conformation and likely better elicit antibodies binding the CR3022, 3A3 and other cryptic epitopes.

Provided herein is an antibody that binds the first reported neutralizing S2 epitope, which is conserved across all highly pathogenic coronavirus strains. HDX mapping and epitope mutagenesis identified the epitope in the hinge at the S2 apex, suggesting 3A3 locks spike in its pre-fusion conformation. The 3A3 antibody binds spike when the S2 domain trimer opens, a state which exists in equilibrium with the S2-closed prefusion state in stabilized spikes. Multiple deployed vaccines utilize versions of the stabilized SARS-CoV-2 spike, and antibodies binding 3A3-like epitopes in the S2 core may be elicited during immunization. This conformationally sensitive epitope suggests 3A3 could be used as a sensitive reagent to assess the quality of spike protein in both academic and industrial settings. Interestingly, 3A3 spike binding and pseudovirus neutralization is enhanced for emerging SARS-CoV-2 spike variants with increased transmissibility, apparently because these variants stabilize the RBDs in the up position required for epitope accessibility.

### II. Definitions

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the inherent variation in the method being employed to determine the value, the variation that exists among the study subjects, or a value that is within 10% of a stated value.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

“Nucleic acid,” “nucleic acid sequence,” “oligonucleotide,” “polynucleotide” or other grammatical equivalents as used herein means at least two nucleotides, either deoxyribonucleotides or ribonucleotides, or analogs thereof, covalently linked together. Polynucleotides are polymers of any length, including, e.g., 20, 50, 100, 200, 300, 500, 1000, 2000, 3000, 5000, 7000, 10,000, etc. A polynucleotide described herein generally contains phosphodiester bonds, although in some cases, nucleic acid analogs are included that may have at least one different linkage, e.g., phosphoramidate, phosphorothioate, phosphorodithioate, or O-methylphophoroamidite linkages, and peptide nucleic acid backbones and linkages. Mixtures of naturally occurring polynucleotides and analogs can be made; alternatively, mixtures of different polynucleotide analogs, and mixtures of naturally occurring polynucleotides and analogs may be made. The following are non-limiting examples of polynucleotides: a gene or gene fragment, exons, introns, messenger RNA (mRNA), transfer RNA, ribosomal RNA, ribozymes, cDNA, cRNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term also includes both double- and single-stranded molecules. Unless otherwise specified or required, the term polynucleotide encompasses both the double-stranded form and each of two complementary single-stranded forms known or predicted to make up the double-stranded form. A polynucleotide is composed of a specific sequence of four nucleotide bases: adenine (A), cytosine (C), guanine (G), thymine (T), and uracil (U) for thymine when the polynucleotide is RNA. Thus, the term “polynucleotide sequence” is the alphabetical representation of a polynucleotide molecule. Unless otherwise indicated, a particular polynucleotide sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues.

The terms “peptide,” “polypeptide” and “protein” used herein refer to polymers of amino acid residues. These terms also apply to amino acid polymers in which one or more amino acid residues is an artificial chemical mimetic of a corresponding naturally occurring amino acid, as well as to naturally occurring amino acid polymers, those containing modified residues, and non-naturally occurring amino acid polymers. In the present case, the term “polypeptide” encompasses an antibody or a fragment thereof.

Other terms used in the fields of recombinant nucleic acid technology, microbiology, immunology, antibody engineering, and molecular and cell biology as used herein will be generally understood by one of ordinary skill in the applicable arts.

### III. Antibodies and Modifications of Antibodies

Provided herein are human monoclonal antibodies having clone-paired CDRs from the heavy and light chains as illustrated in any of Tables 1-3 as well as clone-paired variable regions as illustrated in Tables 4, 6, and 7. Such antibodies may be produced using methods described herein.

TABLE 1
CDRs of heavy and light chain variable sequences of the antibodies as predicted by
IMGT/DomainGapAlign (Ehrenmann et al., 2010; Ehrenmann & Lefranc, 2011).

| | | CDR1 | CDR2 | CDR3 |
|---|---|---|---|---|
| Clone | Chain | (SEQ ID NO:) | (SEQ ID NO:) | (SEQ ID NO:) |
| 3A3 | Heavy | GFNIKDTY | IGPAIGNT | ARYYGSSYYYFDY |
| | | (SEQ ID NO: 1) | (SEQ ID NO: 2) | (SEQ ID NO: 3) |
| | Light | KSLLHSNGNTY | RMS | MQYLEYPLT |
| | | (SEQ ID NO: 4) | (SEQ ID NO: 5) | (SEQ ID NO: 6) |
| AM3A3-3 | Heavy | GFNIKNTY | IGPAIGNT | ARYYGSSYYYLDY |
| | | (SEQ ID NO: 81) | (SEQ ID NO: 2) | (SEQ ID NO: 82) |
| | Light | KSLHHSNGNTY | RMS | MQYLEYPLT |
| | | (SEQ ID NO: 83) | (SEQ ID NO: 5) | (SEQ ID NO: 6) |
| AM3A3-5 | Heavy | GFNIKDTY | IGPAIGNT | ARYYGYAYYYFDY |
| | | (SEQ ID NO: 1) | (SEQ ID NO: 2) | (SEQ ID NO: 84) |
| | Light | KSLLHSNGNTY | RMS | MQYLEYPLT |
| | | (SEQ ID NO: 4) | (SEQ ID NO: 5) | (SEQ ID NO: 6) |
| 4H2 | Heavy | GFTFSSYG | ISSGGSYT | ARDGYGNYVGMDY |
| | | (SEQ ID NO: 7) | (SEQ ID NO: 8) | (SEQ ID NO: 9) |
| | Light | SSVSY | STS | QQWSSNSYT |
| | | (SEQ ID NO: 10) | (SEQ ID NO: | (SEQ ID NO: 12) |
| | | | 11) | |
| 4A5 | Heavy | GYTFTSYW | IDPSNSET | ARLGRY |
| | | (SEQ ID NO: 13) | (SEQ ID NO: 14) | (SEQ ID NO: 15) |
| | Light | QNVGAN | SAS | QQYNSYPYTSGGG |
| | | (SEQ ID NO: 16) | (SEQ ID NO: | (SEQ ID NO: 18) |
| | | | 17) | |

TABLE 2
CDRs of heavy and light chain variable sequences of the antibodies
as predicted byParatome (Kunik et al., 2012a; Kunik et al., 2012b).

| | | CDR1 | CDR2 | CDR3 |
|---|---|---|---|---|
| Clone | Chain | (SEQ ID NO:) | (SEQ ID NO:) | (SEQ ID NO:) |
| 3A3 | Heavy | FNIKDTYIH | WIGRIGPAIGNTIYA | ARYYGSSYYYFDY |
| | | (SEQ ID NO: 19) | (SEQ ID NO: 20) | (SEQ ID NO: 21) |
| | Light | KSLLHSNGNTYLY | LLIYRMSNLAS | MQYLEYPL |
| | | (SEQ ID NO: 22) | (SEQ ID NO: 23) | (SEQ ID NO: 24) |
| AM3A3- | Heavy | FNIKNTYIH | WIGRIGPAIGNTIYA | ARYYGSSYYYLDY |
| 3 | | (SEQ ID NO: 85) | (SEQ ID NO: 20) | (SEQ ID NO: 82) |
| | Light | KSLHHSNGNTYLY | LLIYRMSNLAS | MQYLEYPL |
| | | (SEQ ID NO: 86) | (SEQ ID NO: 23) | (SEQ ID NO: 24) |
| AM3A3- | Heavy | FNIKDTYIH | WIGRIGPAIGNTIYA | ARYYGYAYYYFDY |
| 5 | | (SEQ ID NO: 19) | (SEQ ID NO: 20) | (SEQ ID NO: 84) |
| | Light | KSLLHSNGNTYLY | LLIARMSTLAS | MQYLEYPL |
| | | (SEQ ID NO: 22) | (SEQ ID NO: 87) | (SEQ ID NO: 24) |
| 4H2 | Heavy | FTFSSYGMS | WVATISSGGSYTYY | RDGYGNYVGMDY |
| | | (SEQ ID NO: 25) | (SEQ ID NO: 26) | (SEQ ID NO: 27) |
| | Light | SSVSYMH | LWIYSTSNLAS | QQWSSNSY |
| | | (SEQ ID NO: 28) | (SEQ ID NO: 29) | (SEQ ID NO: 30) |
| 4A5 | Heavy | YTFTSYWMH | WVGMIDPSNSETRL | RLGRY |
| | | (SEQ ID NO: 31) | (SEQ ID NO: 32) | (SEQ ID NO: 33) |
| | Light | QNVGANVA | ALIYSASYRYS | QQYNSYPY |
| | | (SEQ ID NO: 34) | (SEQ ID NO: 35) | (SEQ ID NO: 36) |

TABLE 3
CDRs of heavy and light chain variable sequences of the
antibodies as predicted by Chothia.

| | | CDR1 | CDR2 | CDR3 |
|---|---|---|---|---|
| Clone | Chain | (SEQ ID NO:) | (SEQ ID NO:) | (SEQ ID NO:) |
| 3A3 | Heavy | GFNIKDT | GPAIGN | YYGSSYYYFDY |
| | | (SEQ ID NO: 37) | (SEQ ID NO: 38) | (SEQ ID NO: 39) |
| | Light | RSSKSLLHSNGNTYLY | RMSNLAS | MQYLEYPLT |
| | | (SEQ ID NO: 40) | (SEQ ID NO: 41) | (SEQ ID NO: 42) |
| AM3A3- | Heavy | GFNIKNT | GPAIGN | YYGSSYYYLDY |
| 3 | | (SEQ ID NO: 88) | (SEQ ID NO: 38) | (SEQ ID NO: 89) |
| | Light | RSSKSLHHSNGNTYLY | RMSNLAS | MQYLEYPLT |
| | | (SEQ ID NO: 90) | (SEQ ID NO: 41) | (SEQ ID NO: 42) |
| AM3A3- | Heavy | GFNIKDT | GPAIGN | YYGYAYYYFDY |
| 5 | | (SEQ ID NO: 37) | (SEQ ID NO: 38) | (SEQ ID NO: 91) |
| | Light | RSSKSLLHSNGNTYLY | RMSTLAS | MQYLEYPLT |
| | | (SEQ ID NO: 40) | (SEQ ID NO: 92) | (SEQ ID NO: 42) |
| 4H2 | Heavy | GFTESSY | SSGGSY | DGYGNYVGMDY |
| | | (SEQ ID NO: 43) | (SEQ ID NO: 44) | (SEQ ID NO: 45) |
| | Light | SASSSVSYMH | STSNLAS | QQWSSNSYT |
| | | (SEQ ID NO: 46) | (SEQ ID NO: 47) | (SEQ ID NO: 48) |
| 4A5 | Heavy | GYTFTSY | DPSNSE | LGRY |
| | | (SEQ ID NO: 49) | (SEQ ID NO: 50) | (SEQ ID NO: 51) |
| | Light | KASQNVGANVAWY | SASYRYS | QQYNSYPYT |
| | | (SEQ ID NO: 52) | (SEQ ID NO: 53) | (SEQ ID NO: 54) |

TABLE 4
Amino acid sequences of the full-
length antibody variable regions.

| Clone | Chain | Variable Sequence | SEQ ID NO: |
|---|---|---|---|
| 3A3 | Heavy | MGWSCIILFLVATATGVHSQVQLQQSGAELLKPGASVKLSC | 55 |
| | | TASGFNIKDTYIHWLKQRPEQGLEWIGRIGPAIGNTIYAPK | |
| | | FQGKATITTDTSSNTAYLQLSSLTSEDTAVYYCARYYGSSY | |
| | | YYFDYWGQGTTLTVSSASTKGPSVFPLAPSSKSTSGGTAAL | |
| | | GCLVKDYFPEPVTVSWNSGALTSGVHTFPAVLQSSGLYSLS | |
| | | SVVTVPSSSLGTQTYICNVNHKPSNTKVDKRVEPKSCDKTH | |
| | | TCPPCPAPELLGGPSVFLFPPKPKDTLMISRTPEVTCVVVD | |
| | | VSHEDPEVKFNWYVDGVEVHNAKTKPREEQYNSTYRVVSVL | |
| | | TVLHQDWLNGKEYKCKVSNKALPAPIEKTISKAKGQPREPQ | |
| | | VYTLPPSREEMTKNQVSLTCLVKGFYPSDIAVEWESNGQPE | |
| | | NNYKTTPPVLDSDGSFFLYSKLTVDKSRWQQGNVFSCSVMH | |
| | | EALHNHYTQKSLSLSPGK | |
| | Light | MGWSCIILFLVATATGVHSDIVMTQSAPSVPVTPGESVSIS | 56 |
| | | CRSSKSLLHSNGNTYLYWFLQRPGQSPQLLIYRMSNLASGV | |
| | | PDRFSGSGSGTAFTLRISRVEAEDVGVYYCMQYLEYPLTFG | |
| | | AGTKLELKRTVAAPSVFIFPPSDEQLKSGTASVVCLLNNFY | |
| | | PREAKVQWKVDNALQSGNSQESVTEQDSKDSTYSLSSTLTL | |
| | | SKADYEKHKVYACEVTHQGLSSPVTKSENRGEC | |
| 4H2 | Heavy | MGWSCIILFLVATATGVHSEVQVVESGGGLVQPGGSRKLSC | 57 |
| | | AASGFTFSSYGMSWVRQTPDKRLEWVATISSGGSYTYYPDS | |
| | | VKGRFTISRDNAKNNLYLQMSSLKSEDTAMYYCARDGYGNY | |
| | | VGMDYWGQGTSVTVSSASTKGPSVFPLAPSSKSTSGGTAAL | |
| | | GCLVKDYFPEPVTVSWNSGALTSGVHTFPAVLQSSGLYSLS | |
| | | SVVTVPSSSLGTQTYICNVNHKPSNTKVDKRVEPKSCDKTH | |
| | | TCPPCPAPELLGGPSVFLFPPKPKDTLMISRTPEVTCVVVD | |
| | | VSHEDPEVKFNWYVDGVEVHNAKTKPREEQYNSTYRVVSVL | |
| | | TVLHQDWLNGKEYKCKVSNKALPAPIEKTISKAKGQPREPQ | |
| | | VYTLPPSREEMTKNQVSLTCLVKGFYPSDIAVEWESNGQPE | |
| | | NNYKTTPPVLDSDGSFFLYSKLTVDKSRWQQGNVFSCSVMH | |
| | | EALHNHYTQKSLSLSPGK | |
| | Light | MGWSCIILFLVATATGVHSDIVLTQSPAIMSASPGEKVTIT | 58 |
| | | CSASSSVSYMHWFQQKPGTSPKLWIYSTSNLASGVPARFSG | |
| | | SGSGTSYSLTISSMEAEDAATYYCQQWSSNSYTFGGGTKLE | |
| | | IKRTVAAPSVFIFPPSDEQLKSGTASVVCLLNNFYPREAKV | |
| | | QWKVDNALQSGNSQESVTEQDSKDSTYSLSSTLTLSKADYE | |
| | | KHKVYACEVTHQGLSSPVTKSFNRGEC | |
| 4A5 | Heavy | MGWSCIILFLVATATGVHSQVQLQQSGAELAKPGASVKMSC | 59 |
| | | KASGYTFTSYWMHWVKQRPGQGLEWVGMIDPSNSETRLNQK | |
| | | FKDKATLNVDKSSNTAYMQLSSLTSEDSAVYYCARLGRYWG | |
| | | QGTTLTVSSASTKGPSVFPLAPSSKSTSGGTAALGCLVKDY | |
| | | FPEPVTVSWNSGALTSGVHTFPAVLQSSGLYSLSSVVTVPS | |
| | | SSLGTQTYICNVNHKPSNTKVDKRVEPKSCDKTHTCPPCPA | |
| | | PELLGGPSVFLFPPKPKDTLMISRTPEVTCVVVDVSHEDPE | |
| | | VKFNWYVDGVEVHNAKTKPREEQYNSTYRVVSVLTVLHQDW | |
| | | LNGKEYKCKVSNKALPAPIEKTISKAKGQPREPQVYTLPPS | |
| | | REEMTKNQVSLTCLVKGFYPSDIAVEWESNGQPENNYKTTP | |
| | | PVLDSDGSFFLYSKLTVDKSRWQQGNVFSCSVMHEALHNHY | |
| | | TQKSLSLSPGK | |
| | Light | MGWSCIILFLVATATGVHSDIVMTQSHKFMSTSVGDRVSIT | 60 |
| | | CKASQNVGANVAWYQQKPGQSPKALIYSASYRYSGVPDRFT | |
| | | GSGSGTDFTLTISNVQSEDLAEYFCQQYNSYPYTSGGGTKL | |
| | | EIKRTVAAPSVFIFPPSDEQLKSGTASVVCLLNNFYPREAK | |
| | | VQWKVDNALQSGNSQESVTEQDSKDSTYSLSSTLILSKADY | |
| | | EKHKVYACEVTHQGLSSPVTKSFNRGEC | |

*underlining indicates the signal peptide

TABLE 5
Amino acid sequences of the Fab variable regions.

| Clone | Chain | Variable Sequence | SEQ ID NO: |
|---|---|---|---|
| 3A3 | Heavy | MRPTWAWWLFLVLLLALWAPARGQVQLQQSGAELLKPGASV | 61 |
| | | KLSCTASGFNIKDTYIHWLKQRPEQGLEWIGRIGPAIGNTI | |
| | | YAPKFQGKATITTDTSSNTAYLQLSSLISEDTAVYYCARYY | |
| | | GSSYYYFDYWGQGTTLTVSSASTTPPSVYPLAPGSAAQTNS | |
| | | MVTLGCLVKGYFPEPVTVTWNSGSLSSGVHTFPAVLQSDLY | |
| | | TLSSSVTVPSSTWPSETVTCNVAHPASSTKVDKKIVPRDCG | |
| | | KGLEVLFQ | |
| | Light | MGWSCIILFLVATATGVHSDIVMTQSAPSVPVTPGESVSIS | 62 |
| | | CRSSKSLLHSNGNTYLYWFLQRPGQSPQLLIYRMSNLASGV | |
| | | PDRFSGSGSGTAFTLRISRVEAEDVGVYYCMQYLEYPLTFG | |
| | | AGTKLELKRTADAAPTVSIFPPSSEQLTSGGASVVCFLNNF | |
| | | YPKDINVKWKIDGSERQNGVLNSWTDQDSKDSTYSMSSTLT | |
| | | LTKDEYERHNSYTCEATHKTSTSPIVKSENRNEC | |
| 4H2 | Heavy | MRPTWAWWLFLVLLLALWAPARGEVQVVESGGGLVQPGGSR | 63 |
| | | KLSCAASGFTFSSYGMSWVRQTPDKRLEWVATISSGGSYTY | |
| | | YPDSVKGRFTISRDNAKNNLYLQMSSLKSEDTAMYYCARDG | |
| | | YGNYVGMDYWGQGTSVTVSSASTTPPSVYPLAPGSAAQTNS | |
| | | MVTLGCLVKGYFPEPVTVTWNSGSLSSGVHTFPAVLQSDLY | |
| | | TLSSSVTVPSSTWPSETVTCNVAHPASSTKVDKKIVPRDCG | |
| | | KGLEVLFQ | |
| | Light | MGWSCIILFLVATATGVHSDIVLTQSPAIMSASPGEKVTIT | 64 |
| | | CSASSSVSYMHWFQQKPGTSPKLWIYSTSNLASGVPARFSG | |
| | | SGSGTSYSLTISSMEAEDAATYYCQQWSSNSYTFGGGTKLE | |
| | | IKRTADAAPTVSIFPPSSEQLTSGGASVVCFLNNFYPKDIN | |
| | | VKWKIDGSERQNGVLNSWTDQDSKDSTYSMSSTLTLTKDEY | |
| | | ERHNSYTCEATHKTSTSPIVKSFNRNEC | |
| 4A5 | Heavy | MRPTWAWWLFLVLLLALWAPARGQVQLQQSGAELAKPGASV | 65 |
| | | KMSCKASGYTFTSYWMHWVKQRPGQGLEWVGMIDPSNSETR | |
| | | LNQKFKDKATLNVDKSSNTAYMQLSSLTSEDSAVYYCARLG | |
| | | RYWGQGTTLTVSSASTTPPSVYPLAPGSAAQTNSMVTLGCL | |
| | | VKGYFPEPVTVTWNSGSLSSGVHTFPAVLQSDLYTLSSSVT | |
| | | VPSSTWPSETVTCNVAHPASSTKVDKKIVPRDCGKGLEVLF | |
| | | Q | |
| | Light | MGWSCIILFLVATATGVHSDIVMTQSHKFMSTSVGDRVSIT | 66 |
| | | CKASQNVGANVAWYQQKPGQSPKALIYSASYRYSGVPDRFT | |
| | | GSGSGTDFTLTISNVQSEDLAEYFCQQYNSYPYTSGGGTKL | |
| | | EIKRTADAAPTVSIFPPSSEQLTSGGASVVCFLNNFYPKDI | |
| | | NVKWKIDGSERQNGVLNSWTDQDSKDSTYSMSSTLTLTKDE | |
| | | YERHNSYTCEATHKTSTSPIVKSFNRNEC | |

*underlining indicates the signal peptide

TABLE 6
Amino acid sequences of the humanized 3A3 variable regions.

| Clone | | | SEQ ID |
|---|---|---|---|
| Name | Chain | Variable Sequence | NO: |
| ven3A | Heavy | QVQLVQSGAEVLKPGASVKLSCKASGFNIKDTYIHWLKQAP | 68 |
| 3VH | | GQRLEWIGRIGPAIGNTIYAPKFQGKATITTDTSASTAYLE | |
| | | LSSLRSEDTAVYYCARYYGSSYYYFDYWGQGTTVTVSS | |
| ven3A | Light | DIVMTQSPLSVPVTPGEPVSISCRSSKSLLHSNGNTYLYWF | 69 |
| 3VL | | LQKPGQSPQLLIYRMSNLASGVPDRFSGSGSGTAFTLKISR | |
| | | VEAEDVGVYYCMQYLEYPLTFGAGTKLEIK | |
| cdr3A3 | Heavy | QVQLVQSGAEVKKPGASVKVSCKASGFNIKDTYIHWVRQAP | 70 |
| VH | | GQRLEWIGRIGPAIGNTIYAPKFQGRVTITRDTSASTAYME | |
| | | LSSLRSEDTAVYYCARYYGSSYYYFDYWGQGTTVTVSS | |
| cdr3A3 | Light | DIVMTQSPLSLPVTPGEPASISCRSSKSLLHSNGNTYLYWF | 71 |
| VL | | LQKPGQSPQLLIYRMSNLASGVPDRFSGSGSGTAFTLKISR | |
| | | VEAEDVGVYYCMQYLEYPLTFGAGTKLEIK | |
| sdr3A3 | Heavy | QVQLVQSGAEVKKPGASVKVSCKASGFNIKDTYMHWVRQAP | 72 |
| VH | | GQRLEWIGRIGPAIGNTIYSQKFQGRVTITRDTSASTAYME | |
| | | LSSLRSEDTAVYYCARYYGSSYYYFDYWGQGTTVTVSS | |
| sdr3A3 | Light | DIVMTQSPLSLPVTPGEPASISCRSSKSLLHSNGNTYLYWF | 73 |
| VL | | LQKPGQSPQLLIYRGSNLASGVPDRFSGSGSGTAFTLKISR | |
| | | VEAEDVGVYYCMQYLEYPLTFGAGTKLEIK | |
| abb3A | Heavy | QVQLVQSGAEVKKPGASVKVSCKASGFNIKDTYIHWVRQAP | 74 |
| 3VH | | GQRLEWIGRIGPAIGNTIYSQKFQGRVTITRDISASTAYME | |
| | | LSSLRSEDTAVYYCARYYGSSYYYFDYWGQGTTVTVSS | |

TABLE 7
Amino acid sequences of the affinity
matured 3A3 variable regions.

| | | | SEQ |
|---|---|---|---|
| | | | ID |
| Clone | Chain | Variable Sequence | NO: |
| AM3A | Heavy | QVQLVQSGADVLKPGASVKL | 77 |
| 3-3 | | SCKASGFNIKNTYIHWLKQA | |
| | | PGQRLEWIGRIGPAIGNTIY | |
| | | APKFQGKATITTDTSASTAY | |
| | | LELSSLRSEDTAVYYCARYY | |
| | | GSSYYYLDYWGQGTTVTVSS | |
| | Light | DIVMTQSPLSVPVTPGEPVS | 78 |
| | | ISCRSSKSLHHSNGNTYLYW | |
| | | FLQKPGQSPQLLIYRMSNLA | |
| | | SGVPDRFSGSGNGTAFTLKI | |
| | | SRVEAEDVGVYYCMQYLEYP | |
| | | LTFDAGTKLEIK | |
| AM3A | Heavy | QVQLVQSGAEVLKPGASVKL | 79 |
| 3-5 | | SCKASGFNIKDTYIHWLKQA | |
| | | PGQRLEWIGRIGPAIGNTIY | |
| | | APKFQGKATITTDTSASTAY | |
| | | LELSSLRSEDTAVYYCARYY | |
| | | GYAYYYFDYWGQGTTVTVSS | |
| | Light | DIVMTQSPLSVPVTPGEPVS | 80 |
| | | ISCRSSKSLLHSNGNTYLYW | |
| | | FLQKPGQSPQLLIARMSTLA | |
| | | SGVPDRFSGSGSGTAFTLKI | |
| | | SRVEAEDVGVYYCMQYLEYP | |
| | | LTFGAGTKLEIK | |

The monoclonal antibodies of the present invention have several applications, including the production of diagnostic kits for use in detecting and diagnosing coronavirus infections, as well as for treating or preventing coronavirus infections in patients. In these contexts, one may link such antibodies to diagnostic or therapeutic agents, use them as capture agents or competitors in competitive assays, or use them individually without additional agents being attached thereto. The antibodies may be mutated or modified, as discussed further below. Methods for preparing and characterizing antibodies are well known in the art (see, e.g., Antibodies: A Laboratory Manual, Cold Spring Harbor Laboratory, 1988; U.S. Pat. No. 4,196,265).

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also fragments thereof (such as Fab, Fab′, F(ab′)₂, Fv, Fd, Fd′, single chain antibody (ScFv), diabody, linear antibody), mutants thereof, naturally occurring variants, fusion proteins comprising an antibody portion with an antigen recognition site of the required specificity, humanized antibodies, chimeric antibodies, and any other modified configuration of the immunoglobulin molecule that comprises an antigen recognition site of the required specificity.

An “isolated antibody” is one that has been separated and/or recovered from a component of its natural environment. Contaminant components of its natural environment are materials that would interfere with diagnostic or therapeutic uses for the antibody, and may include enzymes, hormones, and other proteinaceous or non-proteinaceous solutes. In particular instances, the antibody is purified: (1) to greater than 95% by weight of antibody as determined by the Lowry method, and most particularly more than 99% by weight; (2) to a degree sufficient to obtain at least 15 residues of N-terminal or internal amino acid sequence by use of a spinning cup sequenator; or (3) to homogeneity by SDS-PAGE under reducing or non-reducing conditions using Coomassie blue or silver stain. An isolated antibody includes the antibody in situ within recombinant cells since at least one component of the antibody's natural environment will not be present. Ordinarily, however, an isolated antibody will be prepared by at least one purification step.

The basic four-chain antibody unit is a heterotetrameric glycoprotein composed of two identical light (L) chains and two identical heavy (H) chains. The term “heavy chain” as used herein refers to the larger immunoglobulin subunit which associates, through its amino terminal region, with the immunoglobulin light chain. The heavy chain comprises a variable region (V_{H}) and a constant region (C_{H}). The constant region further comprises the C_{H}1, hinge, C_{H}2, and C_{H}3 domains. In the case of IgE, IgM, and IgY, the heavy chain comprises a C_{H}4 domain but does not have a hinge domain. Those skilled in the art will appreciate that heavy chains are classified as gamma, mu, alpha, delta, or epsilon (γ, μ, α, δ, ε), with some subclasses among them (e.g., γ1-γ4, α1-α2). It is the nature of this chain that determines the “class” of the antibody as IgG, IgM, IgA IgD, or IgE, respectively. The immunoglobulin subclasses (isotypes), e.g., IgG1, IgG2, IgG3, IgG4, IgA1, etc. are well characterized and are known to confer functional specialization.

The term “light chain” as used herein refers to the smaller immunoglobulin subunit which associates with the amino terminal region of a heavy chain. As with a heavy chain, a light chain comprises a variable region (V_{L}) and a constant region (C_{L}). Light chains are classified as either kappa or lambda (κ, λ) based on the amino acid sequences of their constant domains (C_{L}). A pair of these can associate with a pair of any of the various heavy chains to form an immunoglobulin molecule. Also encompassed in the meaning of light chain are light chains with a lambda variable region (V-lambda) linked to a kappa constant region (C-kappa) or a kappa variable region (V-kappa) linked to a lambda constant region (C-lambda).

An IgM antibody, for example, consists of 5 basic heterotetramer units along with an additional polypeptide called J chain, and therefore contain 10 antigen binding sites, while secreted IgA antibodies can polymerize to form polyvalent assemblages comprising 2-5 of the basic 4-chain units along with J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable region (V_{H}) followed by three constant domains (C_{H}) for each of the alpha and gamma chains and four C_{H} domains for mu and isotypes. Each L chain has at the N-terminus, a variable region (V_{L}) followed by a constant domain (C_{L}) at its other end. The V_{L} is aligned with the V_{H} and the C_{L} is aligned with the first constant domain of the heavy chain (C_{H}1). Particular amino acid residues are believed to form an interface between the light chain and heavy chain variable regions. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71, and Chapter 6.

A “variable region” of an antibody refers to the variable region of the antibody light chain or the variable region of the antibody heavy chain, either alone or in combination. The term “variable” refers to the fact that certain segments of the variable regions differ extensively in sequence among antibodies. The variable regions of both the light (V_{L}) and heavy (V_{H}) chain portions mediate antigen binding and define the specificity of a particular antibody for its particular antigen. However, the variability is not evenly distributed across the entirety of the variable regions. Instead, the variable regions consist of relatively invariant stretches called framework regions (FRs) separated by shorter regions of extreme variability called complementarity determining regions (CDRs) or hypervariable regions. The variable regions of native heavy and light chains each comprise four FRs, largely adopting a beta-sheet configuration, connected by three CDRs, which form loops connecting, and in some cases forming part of, the beta-sheet structure. The CDRs complement an antigen's shape and determine the antibody's affinity and specificity for the antigen. There are six CDRs in both V_{L} and V_{H}. The CDRs in each chain are held together in close proximity by the FRs and, with the CDRs from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)).

The term “hypervariable region” when used herein refers to the amino acid residues of an antibody that are responsible for antigen binding. The hypervariable region generally comprises amino acid residues from a “complementarity determining region” or “CDR” (e.g., around about residues 24-34 (L1), 50-56 (L2) and 89-97 (L3) in the V_{L}, and around about 31-35 (H1), 50-65 (H2) and 95-102 (H3) in the V_{H} when numbered in accordance with the Kabat numbering system; Kabat et al., Sequences of Proteins of Immunological Interest, 5th Ed. Public Health Service, National Institutes of Health, Bethesda, Md. (1991)); and/or those residues from a “hypervariable loop” (e.g., residues 24-34 (L1), 50-56 (L2) and 89-97 (L3) in the V_{L}, and 26-32 (H1), 52-56 (H2) and 95-101 (H3) in the V_{H} when numbered in accordance with the Chothia numbering system; Chothia and Lesk, J. Mol. Biol. 196:901-917 (1987)); and/or those residues from a “hypervariable loop”/CDR (e.g., residues 27-38 (L1), 56-65 (L2) and 105-120 (L3) in the V_{L}, and 27-38 (H1), 56-65 (H2) and 105-120 (H3) in the V_{H} when numbered in accordance with the IMGT numbering system; Lefranc, M. P. et al. Nucl. Acids Res. 27:209-212 (1999), Ruiz, M. et al. Nucl. Acids Res. 28:219-221 (2000)). Optionally the antibody has symmetrical insertions at one or more of the following points 28, 36 (L1), 63, 74-75 (L2) and 123 (L3) in the V_{L}, and 28, 36 (H1), 63, 74-75 (H2) and 123 (H3) in the V_{sub}H when numbered in accordance with AHo; Honneger, A. and Plunkthun, A. J. Mol. Biol. 309:657-670 (2001)). As used herein, a CDR may refer to CDRs defined by any of these numbering approaches or by a combination of approaches or by other desirable approaches. In addition, a new definition of highly conserved core, boundary and hyper-variable regions can be used.

A “constant region” of an antibody refers to the constant region of the antibody light chain or the constant region of the antibody heavy chain, either alone or in combination. The constant regions of the light chain (CL) and the heavy chain (C_{H}1, C_{H}2 or C_{H}3, or C_{H}4 in the case of IgM and IgE) confer important biological properties such as secretion, transplacental mobility, Fc receptor binding, complement binding, and the like. By convention the numbering of the constant region domains increases as they become more distal from the antigen binding site or amino-terminus of the antibody. The constant regions are not involved directly in binding an antibody to an antigen, but exhibit various effector functions, such as participation of the antibody in antibody dependent cellular cytotoxicity (ADCC), antibody-dependent cellular phagocytosis (ADCP), antibody-dependent neutrophil phagocytosis (ADNP), and antibody-dependent complement deposition (ADCD).

The antibody may be an antibody fragment. “Antibody fragments” comprise only a portion of an intact antibody, generally including an antigen binding site of the intact antibody and thus retaining the ability to bind antigen. Examples of antibody fragments encompassed by the present definition include: (i) the Fab fragment, having V_{L}, C_{L}, V_{H} and C_{H}1 domains; (ii) the Fab′ fragment, which is a Fab fragment having one or more cysteine residues at the C-terminus of the C_{H}1 domain; (iii) the Fd fragment having V_{H} and C_{H}1 domains; (iv) the Fd′ fragment having V_{H} and C_{H}1 domains and one or more cysteine residues at the C-terminus of the C_{H}1 domain; (v) the Fv fragment having the V_{L} and V_{H} domains of a single antibody; (vi) the dAb fragment which consists of a V_{H} domain; (vii) isolated CDR regions; (viii) F(ab′)₂ fragments, a bivalent fragment including two Fab′ fragments linked by a disulfide bridge at the hinge region; (ix) single chain antibody molecules (e.g. single chain Fv; scFv); (x) “diabodies” with two antigen binding sites, comprising a heavy chain variable domain (V_{H}) connected to a light chain variable domain (VL) in the same polypeptide chain; (xi) “linear antibodies” comprising a pair of tandem Fd segments (V_{H}—CH1-V_{H}-C_{H}1) which, together with complementary light chain polypeptides, form a pair of antigen binding regions.

The antibody may be a chimeric antibody. “Chimeric antibodies” refers to those antibodies wherein one portion of each of the amino acid sequences of heavy and light chains is homologous to corresponding sequences in antibodies derived from a particular species or belonging to a particular class, while the remaining segment of the chains is homologous to corresponding sequences in another. For example, a chimeric antibody may be an antibody comprising antigen binding sequences from a non-human donor grafted to a heterologous non-human, human, or humanized sequence (e.g., framework and/or constant domain sequences). Typically, in these chimeric antibodies, the variable region of both light and heavy chains mimics the variable regions of antibodies derived from one species of mammals, while the constant portions are homologous to the sequences in antibodies derived from another. For example, methods have been developed to replace light and heavy chain constant domains of a monoclonal antibody with analogous domains of human origin, leaving the variable regions of the foreign antibody intact. Alternatively, “fully human” monoclonal antibodies are produced in mice transgenic for human immunoglobulin genes. Methods have also been developed to convert variable domains of monoclonal antibodies to more human form by recombinantly constructing antibody variable domains having both rodent, for example, mouse, and human amino acid sequences. In “humanized” monoclonal antibodies, only the hypervariable CDR is derived from mouse monoclonal antibodies, and the framework and constant regions are derived from human amino acid sequences (see U.S. Pat. Nos. 5,091,513 and 6,881,557, incorporated herein by reference). It is thought that replacing amino acid sequences in the antibody that are characteristic of rodents with amino acid sequences found in the corresponding position of human antibodies will reduce the likelihood of adverse immune reaction during therapeutic use. A hybridoma or other cell producing an antibody may also be subject to genetic mutation or other changes, which may or may not alter the binding specificity of antibodies produced by the hybridoma.

Methods for reducing or eliminating the antigenicity of antibodies and antibody fragments are known in the art. When the antibodies are to be administered to a human, the antibodies preferably are “humanized” to reduce or eliminate antigenicity in humans. Preferably, each humanized antibody has the same or substantially the same affinity for the antigen as the non-humanized mouse antibody from which it was derived.

In one humanization approach, chimeric proteins are created in which mouse immunoglobulin constant regions are replaced with human immunoglobulin constant regions. See, e.g., Morrison et al., 1984, PROC. NAT. ACAD. SCI. 81:6851-6855, Neuberger et al., 1984, NATURE 312:604-608; U.S. Pat. No. 6,893,625 (Robinson); U.S. Pat. No. 5,500,362 (Robinson); and U.S. Pat. No. 4,816,567 (Cabilly).

In an approach known as CDR grafting, the CDRs of the light and heavy chain variable regions are grafted into frameworks from another species. For example, murine CDRs can be grafted into human FRs. In some embodiments, the CDRs of the light and heavy chain variable regions of an antibody are grafted into human FRs or consensus human FRs. To create consensus human FRs, FRs from several human heavy chain or light chain amino acid sequences are aligned to identify a consensus amino acid sequence. CDR grafting is described in U.S. Pat. No. 7,022,500 (Queen); U.S. Pat. No. 6,982,321 (Winter); U.S. Pat. No. 6,180,370 (Queen); U.S. Pat. No. 6,054,297 (Carter); U.S. Pat. No. 5,693,762 (Queen); U.S. Pat. No. 5,859,205 (Adair); U.S. Pat. No. 5,693,761 (Queen); U.S. Pat. No. 5,565,332 (Hoogenboom); U.S. Pat. No. 5,585,089 (Queen); U.S. Pat. No. 5,530,101 (Queen); Jones et al. (1986) NATURE 321: 522-525; Riechmann et al. (1988) NATURE 332: 323-327; Verhoeyen et al. (1988) SCIENCE 239: 1534-1536; and Winter (1998) FEBS LETT 430: 92-94.

In an approach called “SUPERHUMANIZATION™” human CDR sequences are chosen from human germline genes, based on the structural similarity of the human CDRs to those of the mouse antibody to be humanized. See, e.g., U.S. Pat. No. 6,881,557 (Foote); and Tan et al., 2002, J. IMMUNOL. 169:1119-1125.

Other methods to reduce immunogenicity include “reshaping,” “hyperchimerization,” and “veneering/resurfacing.” See, e.g., Vaswami et al., 1998, ANNALS OF ALLERGY, ASTHMA, & IMMUNOL. 81:105; Roguska et al., 1996, PROT. ENGINEER 9:895-904; and U.S. Pat. No. 6,072,035 (Hardman). In the veneering/resurfacing approach, the surface accessible amino acid residues in the murine antibody are replaced by amino acid residues more frequently found at the same positions in a human antibody. This type of antibody resurfacing is described, e.g., in U.S. Pat. No. 5,639,641 (Pedersen).

Another approach for converting a mouse antibody into a form suitable for medical use in humans is known as ACTIVMAB™ technology (Vaccinex, Inc., Rochester, NY), which involves a vaccinia virus-based vector to express antibodies in mammalian cells. High levels of combinatorial diversity of IgG heavy and light chains can be produced. See, e.g., U.S. Pat. No. 6,706,477 (Zauderer); U.S. Pat. No. 6,800,442 (Zauderer); and U.S. Pat. No. 6,872,518 (Zauderer). Another approach for converting a mouse antibody into a form suitable for use in humans is technology practiced commercially by KaloBios Pharmaceuticals, Inc. (Palo Alto, CA). This technology involves the use of a proprietary human “acceptor” library to produce an “epitope focused” library for antibody selection. Another approach for modifying a mouse antibody into a form suitable for medical use in humans is HUMAN ENGINEERING™ technology, which is practiced commercially by XOMA (US) LLC. See, e.g., International (PCT) Publication No. WO 93/11794 and U.S. Pat. No. 5,766,886 (Studnicka); U.S. Pat. No. 5,770,196 (Studnicka); U.S. Pat. No. 5,821,123 (Studnicka); and U.S. Pat. No. 5,869,619 (Studnicka).

Any suitable approach, including any of the above approaches, can be used to reduce or eliminate human immunogenicity of an antibody.

### B. Monoclonal Antibodies

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, monoclonal antibodies are advantageous in that they may be synthesized uncontaminated by other antibodies. The modifier “monoclonal” is not to be construed as requiring production of the antibody by any particular method. For example, the monoclonal antibodies useful in the present disclosure may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567) after single cell sorting of an antigen specific B cell, an antigen specific plasmablast responding to an infection or immunization, or capture of linked heavy and light chains from single cells in a bulk sorted antigen specific collection. The monoclonal antibodies may also be isolated from phage antibody libraries using the techniques described in Clackson et al., Nature, 352:624-628 (1991) and Marks et al., J. Mol. Biol., 222:581-597 (1991), for example.

Methods for producing monoclonal antibodies of various types, including humanized, chimeric, and fully human, are well known in the art and highly predictable. For example, the following U.S. patents and patent applications provide enabling descriptions of such methods: U.S. Patent Application Nos. 2004/0126828 and 2002/0172677; and U.S. Pat. Nos. 3,817,837; 3,850,752; 3,939,350; 3,996,345; 4,196,265; 4,275,149; 4,277,437; 4,366,241; 4,469,797; 4,472,509; 4,606,855; 4,703,003; 4,742,159; 4,767,720; 4,816,567; 4,867,973; 4,938,948; 4,946,778; 5,021,236; 5,164,296; 5,196,066; 5,223,409; 5,403,484; 5,420,253; 5,565,332; 5,571,698; 5,627,052; 5,656,434; 5,770,376; 5,789,208; 5,821,337; 5,844,091; 5,858,657; 5,861,155; 5,871,907; 5,969,108; 6,054,297; 6,165,464; 6,365,157; 6,406,867; 6,709,659; 6,709,873; 6,753,407; 6,814,965; 6,849,259; 6,861,572; 6,875,434; and 6,891,024, each incorporated herein by reference.

In the case of human antibodies against natural pathogens, a suitable approach is to identify subjects that have been exposed to the pathogens, such as those who have been diagnosed as having contracted the disease, or those who have been vaccinated to generate protective immunity against the pathogen or to test the safety or efficacy of an experimental vaccine. Circulating anti-pathogen antibodies can be detected, and antibody encoding or producing B cells from the antibody-positive subject may then be obtained.

### C. Single Chain Antibodies

A single chain variable fragment (scFv) is a fusion of the variable regions of the heavy and light chains of immunoglobulins, linked together with a short linker. This chimeric molecule retains the specificity of the original immunoglobulin, despite removal of the constant regions and the introduction of a linker peptide. This modification usually leaves the specificity unaltered. scFv can be created directly from subcloned heavy and light chains derived from a hybridoma or B cell. Single chain variable fragments lack the constant Fc region found in complete antibody molecules, and thus, the common binding sites (e.g., protein A/G) used to purify antibodies. These fragments can often be purified/immobilized using Protein L since Protein L interacts with the variable region of kappa light chains.

Flexible linkers generally are comprised of helix- and turn-promoting amino acid residues such as alanine, serine and glycine. However, other residues can function as well. For example, the linker may have a proline residue two residues after the V_{H} C terminus and an abundance of arginines and prolines at other positions.

A single-chain antibody may also be created by joining receptor light and heavy chains using a non-peptide linker or chemical unit. Generally, the light and heavy chains will be produced in distinct cells, purified, and subsequently linked together in an appropriate fashion (i.e., the N-terminus of the heavy chain being attached to the C-terminus of the light chain via an appropriate chemical bridge).

Cross-linking reagents are used to form molecular bridges that tie functional groups of two different molecules, e.g., a stabilizing and coagulating agent. However, it is contemplated that dimers or multimers of the same analog or heteromeric complexes comprised of different analogs can be created. To link two different compounds in a step-wise manner, hetero-bifunctional cross-linkers can be used that eliminate unwanted homopolymer formation.

An exemplary hetero-bifunctional cross-linker contains two reactive groups: one reacting with primary amine group (e.g., N-hydroxy succinimide) and the other reacting with a thiol group (e.g., pyridyl disulfide, maleimides, halogens, etc.). Through the primary amine reactive group, the cross-linker may react with the lysine residue(s) of one protein (e.g., the selected antibody or fragment) and through the thiol reactive group, the cross-linker, already tied up to the first protein, reacts with the cysteine residue (free sulfhydryl group) of the other protein (e.g., the selective agent).

It is preferred that a cross-linker having reasonable stability in blood will be employed. Numerous types of disulfide-bond containing linkers are known that can be successfully employed to conjugate targeting and therapeutic/preventative agents. Linkers that contain a disulfide bond that is sterically hindered may prove to give greater stability in vivo, preventing release of the targeting peptide prior to reaching the site of action. These linkers are thus one group of linking agents.

For example, SMPT is a bifunctional cross-linker containing a disulfide bond that is “sterically hindered” by an adjacent benzene ring and methyl groups. It is believed that steric hindrance of the disulfide bond serves a function of protecting the bond from attack by thiolate anions such as glutathione which can be present in tissues and blood, and thereby help in preventing decoupling of the conjugate prior to the delivery of the attached agent to the target site. The SMPT cross-linking reagent, as with many other known cross-linking reagents, lends the ability to cross-link functional groups such as the SH of cysteine or primary amines (e.g., the epsilon amino group of lysine). Another possible type of cross-linker includes the hetero-bifunctional photoreactive phenylazides containing a cleavable disulfide bond such as sulfosuccinimidyl-2-(p-azido salicylamido) ethyl-1,3′-dithiopropionate. The N-hydroxy-succinimidyl group reacts with primary amino groups and the phenylazide (upon photolysis) reacts non-selectively with any amino acid residue.

In addition to hindered cross-linkers, non-hindered linkers also can be employed in accordance herewith. Other useful cross-linkers, not considered to contain or generate a protected disulfide, include SATA, SPDP and 2-iminothiolane. The use of such cross-linkers is well understood in the art. Flexible linkers may also be used.

U.S. Pat. No. 4,680,338, describes bifunctional linkers useful for producing conjugates of ligands with amine-containing polymers and/or proteins, especially for forming antibody conjugates with chelators, drugs, enzymes, detectable labels and the like. U.S. Pat. Nos. 5,141,648 and 5,563,250 disclose cleavable conjugates containing a labile bond that is cleavable under a variety of mild conditions. This linker is particularly useful in that the agent of interest may be bonded directly to the linker, with cleavage resulting in release of the active agent. Particular uses include adding a free amino or free sulfhydryl group to a protein, such as an antibody, or a drug.

U.S. Pat. No. 5,856,456 provides peptide linkers for use in connecting polypeptide constituents to make fusion proteins, e.g., single chain antibodies. The linker is up to about 50 amino acids in length, contains at least one occurrence of a charged amino acid (preferably arginine or lysine) followed by a proline, and is characterized by greater stability and reduced aggregation. U.S. Pat. No. 5,880,270 discloses aminooxy-containing linkers useful in a variety of immunodiagnostic and separative techniques.

### D. Bispecific and Multispecific Antibodies

Antibodies may be bispecific or multispecific. “Bispecific antibodies” are antibodies that have binding specificities for at least two different epitopes. Exemplary bispecific antibodies may bind to two different epitopes of a single antigen. Other such antibodies may combine a first antigen binding site with a binding site for a second antigen. Alternatively, an antigen-specific arm may be combined with an arm that binds to a triggering molecule on a leukocyte, such as a T-cell receptor molecule (e.g., CD3), or Fc receptors for IgG (FcγR), such as FcγRI (CD64), FcγRII (CD32) and Fc gamma RIII (CD16), so as to focus and localize cellular defense mechanisms to the infected cell. Bispecific antibodies may also be used to localize cytotoxic agents to infected cells. These antibodies possess an antigen-binding arm and an arm that binds the cytotoxic agent (e.g., saporin, anti-interferon-α, vinca alkaloid, ricin A chain, methotrexate or radioactive isotope hapten). Bispecific antibodies can be prepared as full-length antibodies or antibody fragments (e.g., F(ab′)₂ bispecific antibodies). Taki et al. (2015) describes a bispecific anti-HSP70/anti-CD3 antibody.

Methods for making bispecific antibodies are known in the art. Traditional production of full-length bispecific antibodies is based on the co-expression of two immunoglobulin heavy chain-light chain pairs, where the two chains have different specificities. Because of the random assortment of immunoglobulin heavy and light chains, these hybridomas (quadromas) produce a potential mixture of ten different antibody molecules, of which only one has the correct bispecific structure. Purification of the correct molecule, which is usually done by affinity chromatography steps, is rather cumbersome, and the product yields are low.

According to a different approach, antibody variable regions with the desired binding specificities (antibody-antigen combining sites) are fused to immunoglobulin constant domain sequences. Preferably, the fusion is with an Ig heavy chain constant domain, comprising at least part of the hinge, C_{H2}, and C_{H3} regions. It is preferred to have the first heavy-chain constant region (C_{H1}) containing the site necessary for light chain bonding, present in at least one of the fusions. DNAs encoding the immunoglobulin heavy chain fusions and, if desired, the immunoglobulin light chain, are inserted into separate expression vectors, and are co-transfected into a suitable host cell. This provides for greater flexibility in adjusting the mutual proportions of the three polypeptide fragments when unequal ratios of the three polypeptide chains used in the construction provide the optimum yield of the desired bispecific antibody. It is, however, possible to insert the coding sequences for two or all three polypeptide chains into a single expression vector when the expression of at least two polypeptide chains in equal ratios results in high yields or when the ratios have no significant effect on the yield of the desired chain combination.

The bispecific antibodies may be composed of a hybrid immunoglobulin heavy chain with a first binding specificity in one arm, and a hybrid immunoglobulin heavy chain-light chain pair (providing a second binding specificity) in the other arm. This asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986).

According to another approach described in U.S. Pat. No. 5,731,168, the interface between a pair of antibody molecules can be engineered to maximize the percentage of heterodimers that are recovered from recombinant cell culture. The preferred interface comprises at least a part of the C_{H3} domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science, 229: 81 (1985) describe a procedure wherein intact antibodies are proteolytically cleaved to generate F(ab′)₂ fragments. These fragments are reduced in the presence of the dithiol complexing agent, sodium arsenite, to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Techniques exist that facilitate the direct recovery of Fab′-SH fragments from E. coli, which can be chemically coupled to form bispecific antibodies. Shalaby et al., J. Exp. Med., 175: 217-225 (1992) describe the production of a humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described (Merchant et al., Nat. Biotechnol. 16, 677-681 (1998)). For example, bispecific antibodies have been produced using leucine zippers (Kostelny et al., J. Immunol., 148(5):1547-1553, 1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a V_{H} connected to a V_{L} by a linker that is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

A bispecific or multispecific antibody may be formed as a DOCK-AND-LOCK™ (DNL™) complex (see, e.g., U.S. Pat. Nos. 7,521,056; 7,527,787; 7,534,866; 7,550,143 and 7,666,400). Generally, the technique takes advantage of the specific and high-affinity binding interactions that occur between a dimerization and docking domain (DDD) sequence of the regulatory (R) subunits of cAMP-dependent protein kinase (PKA) and an anchor domain (AD) sequence derived from any of a variety of AKAP proteins (Baillie et al., FEBS Letters. 2005; 579: 3264; Wong and Scott, Nat. Rev. Mol. Cell Biol. 2004; 5: 959). The DDD and AD peptides may be attached to any protein, peptide or other molecule. Because the DDD sequences spontaneously dimerize and bind to the AD sequence, the technique allows the formation of complexes between any selected molecules that may be attached to DDD or AD sequences.

Antibodies with more than two valencies are contemplated. For example, trispecific antibodies can be prepared (Tutt et al., J. Immunol. 147: 60, 1991; Xu et al., Science, 358(6359):85-90, 2017). The antibodies may also involve sequences or moieties that permit dimerization or multimerization of the receptors. Such sequences include those derived from IgA, which permit formation of multimers in conjunction with the J-chain. Another multimerization domain is the Gal4 dimerization domain.

A multivalent antibody may be internalized (and/or catabolized) faster than a bivalent antibody by a cell expressing an antigen to which the antibody binds. The antibodies of the present disclosure can be multivalent antibodies with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises (or consists of) an Fc region or a hinge region. In this scenario, the antibody will comprise an Fc region and three or more antigen binding sites amino-terminal to the Fc region. Multivalent antibodies may comprise (or consist of) three to about eight, for example four, antigen binding sites. The multivalent antibody comprises at least one polypeptide chain (and preferably two polypeptide chains), wherein the polypeptide chain(s) comprise two or more variable regions. For instance, the polypeptide chain(s) may comprise VD1-(X1).sub.n-VD2-(X2)ₙ-Fc, wherein VD1 is a first variable region, VD2 is a second variable region, Fc is one polypeptide chain of an Fc region, X1 and X2 represent an amino acid or polypeptide, and n is 0 or 1. For instance, the polypeptide chain(s) may comprise: VH-CH1-flexible linker-VH-CH1-Fc region chain; or VH-CH1-VH-CH1-Fc region chain. The multivalent antibody herein may further comprise at least two (and preferably four) light chain variable region polypeptides. The multivalent antibody herein may, for instance, comprise from about two to about eight light chain variable region polypeptides. The light chain variable region polypeptides contemplated here comprise a light chain variable region and, optionally, further comprise a C_{L} domain.

Charge modifications are particularly useful in the context of a multispecific antibody, where amino acid substitutions in Fab molecules result in reducing the mispairing of light chains with non-matching heavy chains (Bence-Jones-type side products), which can occur in the production of Fab-based bi-/multispecific antigen binding molecules with a VH/VL exchange in one (or more, in case of molecules comprising more than two antigen-binding Fab molecules) of their binding arms (see also PCT publication no. WO 2015/150447, particularly the examples therein, incorporated herein by reference in its entirety).

### E. BiTES

A bi-specific T-cell engagers (BiTE®) is an artificial bispecific monoclonal antibody that directs a host's immune system, more specifically the T cells' cytotoxic activity, to target diseased cells. BiTEs are fusion proteins consisting of two single-chain variable fragments (scFvs) of different antibodies, or amino acid sequences from four different genes, on a single peptide chain of about 55 kilodaltons. One of the scFvs binds to T cells via the CD3 receptor, and the other to an infected cell via a specific molecule.

Like other bispecific antibodies, and unlike ordinary monoclonal antibodies, BiTEs form a link between T cells and target cells. This causes T cells to exert cytotoxic activity on target cells by producing proteins like perforin and granzymes, independently of the presence of MHC I or co-stimulatory molecules. These proteins enter the target cells and initiate apoptosis. This action mimics physiological processes observed during T cell attacks against infected cells.

### F. Antibody Conjugates

Antibodies of the present disclosure may be linked to at least one agent to form an antibody conjugate. The conjugate can be, for example, an antibody conjugated to another proteinaceous, carbohydrate, lipid, or mixed moiety molecule(s). Such antibody conjugates include, but are not limited to, modifications that include linking the antibody to one or more polymers. For example, an antibody may be linked to one or more water-soluble polymers. Linkage to a water-soluble polymer reduces the likelihood that the antibody will precipitate in an aqueous environment, such as a physiological environment. One skilled in the art can select a suitable water-soluble polymer based on considerations including, but not limited to, whether the polymer/antibody conjugate will be used in the treatment of a patient and, if so, the pharmacological profile of the antibody (e.g., half-life, dosage, activity, antigenicity, and/or other factors).

In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules which have been attached to antibodies include toxins, anti-tumor agents, therapeutic enzymes, radionuclides, antiviral agents, chelating agents, cytokines, growth factors, and oligo- or polynucleotides. By contrast, a reporter molecule is defined as any moiety which may be detected using an assay. Non-limiting examples of reporter molecules which have been conjugated to antibodies include enzymes, radiolabels, haptens, fluorescent labels, phosphorescent molecules, chemiluminescent molecules, chromophores, photoaffinity molecules, colored particles or ligands, an enzyme (e.g., that catalyzes a colorimetric or fluorometric reaction), a substrate, a solid matrix, such as biotin. An antibody may comprise one, two, or more of any of these labels.

Antibody conjugates may be used to deliver cytotoxic agents to target cells. Cytotoxic agents of this type may improve antibody-mediated cytotoxicity, and include such moieties as cytokines that directly or indirectly stimulate cell death, radioisotopes, chemotherapeutic drugs (including prodrugs), bacterial toxins (e.g., pseudomonas exotoxin, diphtheria toxin, etc.), plant toxins (e.g., ricin, gelonin, etc.), chemical conjugates (e.g., maytansinoid toxins, auristatins, α-amanitin, anthracyclines, calechaemicin, etc.), radioconjugates, enzyme conjugates (e.g., RNase conjugates, granzyme antibody-directed enzyme/prodrug therapy), and the like.

Antibody conjugates are also used as diagnostic agents. Antibody diagnostics generally fall within two classes, those for use in in vitro diagnostics, such as in a variety of immunoassays, and those for use in vivo diagnostic protocols, generally known as “antibody-directed imaging.” Many appropriate imaging agents are known in the art, as are methods for their attachment to antibodies (see, for e.g., U.S. Pat. Nos. 5,021,236, 4,938,948, and 4,472,509). The imaging moieties used can be paramagnetic ions, radioactive isotopes, fluorochromes, NMR-detectable substances, and X-ray imaging agents.

The paramagnetic ions contemplated for use as conjugates include chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and bismuth (III).

The radioactive isotopes contemplated for use as conjugated include astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} and/or yttrium⁹⁰. ¹²⁵I is often being preferred. Technicium^{99m} and/or indium¹¹¹ are also often preferred due to their low energy and suitability for long range detection. Radioactively labeled monoclonal antibodies of the present disclosure may be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies according to the disclosure may be labeled with technetium^{99m} by ligand exchange process, for example, by reducing pertechnate with stannous solution, chelating the reduced technetium onto a Sephadex column and applying the antibody to this column. Alternatively, direct labeling techniques may be used, e.g., by incubating pertechnate, a reducing agent such as SNCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) or ethylene diaminetetracetic acid (EDTA).

The fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, tetramethylrhodamine, and/or Texas Red.

Additional types of antibodies contemplated in the present disclosure are those intended primarily for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase or glucose oxidase. Preferred secondary binding ligands are biotin and avidin and streptavidin compounds.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to the antibody (U.S. Pat. Nos. 4,472,509 and 4,938,948). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate.

Another known method of site-specific attachment of molecules to antibodies comprises the reaction of antibodies with hapten-based affinity labels. Essentially, hapten-based affinity labels react with amino acids in the antigen binding site, thereby destroying this site and blocking specific antigen reaction. However, this may not be advantageous since it results in loss of antigen binding by the antibody conjugate.

Molecules containing azido groups may also be used to form covalent bonds to proteins through reactive nitrene intermediates that are generated by low intensity ultraviolet light. In particular, 2- and 8-azido analogues of purine nucleotides have been used as site-directed photoprobes to identify nucleotide binding proteins in crude cell extracts. The 2- and 8-azido nucleotides have also been used to map nucleotide binding domains of purified proteins and may be used as antibody binding agents.

Derivatization of immunoglobulins by selectively introducing sulfhydryl groups in the Fc region of an immunoglobulin, using reaction conditions that do not alter the antibody combining site are also contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fc region have also been disclosed in the literature. This approach has been reported to produce diagnostically and therapeutically promising antibodies which are currently in clinical evaluation.

### G. Antibody Drug Conjugates

Antibody drug conjugates, or ADCs, are a class of highly potent biopharmaceutical drugs designed as a targeted therapy for the treatment of people with disease. ADCs are complex molecules composed of an antibody (a whole mAb or an antibody fragment, such as a scFv) linked, via a stable chemical linker with labile bonds, to a biological active cytotoxic/anti-viral payload or drug. Antibody drug conjugates are examples of bioconjugates and immunoconjugates.

By combining the unique targeting capabilities of monoclonal antibodies with the cancer-killing ability of cytotoxic drugs, antibody-drug conjugates allow sensitive discrimination between healthy and diseased tissue. This means that, in contrast to traditional systemic approaches, antibody-drug conjugates target and attack the diseased cell so that healthy cells are less severely affected.

In the development ADC-based anti-tumor therapies, an anticancer drug (e.g., a cell toxin or cytotoxin) is coupled to an antibody that specifically targets a certain cell marker (e.g., a protein that, ideally, is only to be found in or on diseased cells). Antibodies track these proteins down in the body and attach themselves to the surface of the diseased cells. The biochemical reaction between the antibody and the target protein (antigen) triggers a signal in the targeted cell, which then absorbs or internalizes the antibody together with the cytotoxin. After the ADC is internalized, the cytotoxic drug is released and kills the cell or impairs cellular replication. Due to this targeting, ideally the drug has lower side effects and gives a wider therapeutic window than other agents.

A stable link between the antibody and cytotoxic agent is a crucial aspect of an ADC. Linkers are based on chemical motifs including disulfides, hydrazones or peptides (cleavable), or thioethers (noncleavable) and control the distribution and delivery of the cytotoxic agent to the target cell. Cleavable and noncleavable types of linkers have been proven to be safe in preclinical and clinical trials. Brentuximab vedotin includes an enzyme-sensitive cleavable linker that delivers the potent and highly toxic antimicrotubule agent Monomethyl auristatin E or MMAE, a synthetic antineoplastic agent, to human specific CD30-positive malignant cells. Because of its high toxicity MMAE, which inhibits cell division by blocking the polymerization of tubulin, cannot be used as a single-agent chemotherapeutic drug. However, the combination of MMAE linked to an anti-CD30 monoclonal antibody (cAC10, a cell membrane protein of the tumor necrosis factor or TNF receptor) proved to be stable in extracellular fluid, cleavable by cathepsin and safe for therapy. Trastuzumab emtansine, the other approved ADC, is a combination of the microtubule-formation inhibitor mertansine (DM-1), a derivative of the Maytansine, and antibody trastuzumab (Herceptin®/Genentech/Roche) attached by a stable, non-cleavable linker.

The availability of better and more stable linkers has changed the function of the chemical bond. The type of linker, cleavable or noncleavable, lends specific properties to the cytotoxic (e.g., anti-cancer) drug. For example, a non-cleavable linker keeps the drug within the cell. As a result, the entire antibody, linker, and cytotoxic agent enter the targeted cell where the antibody is degraded to the level of amino acids. The resulting complex—amino acid, linker and cytotoxic agent—now becomes the active drug. In contrast, cleavable linkers are catalyzed by enzymes in the host cell, thereby releasing the cytotoxic agent.

Another type of cleavable linker adds an extra molecule between the cytotoxic drug and the cleavage site. This linker technology allows researchers to create ADCs with more flexibility without worrying about changing cleavage kinetics. Researchers are also developing a new method of peptide cleavage based on Edman degradation. Future direction in the development of ADCs also include the development of site-specific conjugation (TDCs) to further improve stability and therapeutic index and α-emitting immunoconjugates and antibody-conjugated nanoparticles.

### H. Intrabodies

In a particular embodiment, the antibody is a recombinant antibody that is suitable for action inside of a cell—such antibodies are known as “intrabodies.” These antibodies may interfere with target function by a variety of mechanisms, such as by altering intracellular protein trafficking, interfering with enzymatic function, and blocking protein-protein or protein-DNA interactions. In many ways, their structures mimic or parallel those of single chain and single domain antibodies, discussed above. Indeed, single-transcript/single-chain is an important feature that permits intracellular expression in a target cell, and also makes protein transit across cell membranes more feasible. However, additional features are required. An additional feature that intrabodies may require is a signal for intracellular targeting. Vectors that can target intrabodies (or other proteins) to subcellular regions such as the cytoplasm, nucleus, mitochondria and ER have been designed and are commercially available (Invitrogen Corp.).

The two major issues impacting the implementation of intrabody therapeutics are delivery, including cell/tissue targeting, and stability. With respect to delivery, a variety of approaches have been employed, such as tissue-directed delivery, use of cell-type specific promoters, viral-based delivery, use of cell-permeability/membrane translocating peptides, and delivery using exosomes. One means of delivery comprises the use of lipid-based nanoparticles, or exosomes, as taught in U.S. Pat. Appln. Publn. 2018/0177727, which is incorporated by reference here in its entirety. With respect to stability, the approach is generally to either screen by brute force, including methods that involve phage display and may include sequence maturation or development of consensus sequences, or more directed modifications such as insertion stabilizing sequences (e.g., Fc regions, chaperone protein sequences, leucine zippers) and disulfide replacement/modification.

### I. Production and Purification of Antibodies

The methods for generating monoclonal antibodies generally begin along the same lines as those for preparing polyclonal antibodies. The first step for both of these methods is immunization of an appropriate host. As is well known in the art, a given composition for immunization may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimyde and bis-biazotized benzidine. As also is well known in the art, the immunogenicity of a particular immunogen composition can be enhanced by the use of non-specific stimulators of the immune response, known as adjuvants. Exemplary and preferred adjuvants in animals include complete Freund's adjuvant (a non-specific stimulator of the immune response containing killed Mycobacterium tuberculosis), incomplete Freund's adjuvants and aluminum hydroxide adjuvant and in humans include alum, CpG, MFP59, and combinations of immunostimulatory molecules (“Adjuvant Systems”, such as AS01 or AS03). Additional experimental forms of inoculation to induce antigen-specific B cells are possible, including nanoparticle vaccines, or gene-encoded antigens delivered as DNA or RNA genes in a physical delivery system (such as lipid nanoparticle or on a gold biolistic bead), and delivered with needle, gene gun, or transcutaneous electroporation device. The antigen gene also can be carried as encoded by a replication competent or defective viral vector such as adenovirus, adeno-associated virus, poxvirus, herpesvirus, or alphavirus replicon, or alternatively a virus-like particle.

Methods for generating hybrids of antibody-producing spleen or lymph node cells and myeloma cells usually comprise mixing somatic cells with myeloma cells in a 2:1 proportion, though the proportion may vary from about 20:1 to about 1:1, in the presence of an agent or agents (chemical or electrical) that promote the fusion of cell membranes. In some cases, transformation of human B cells with Epstein Barr virus (EBV) as an initial step increases the size of the B cells, enhancing fusion with the relatively large-sized myeloma cells. Transformation efficiency by EBV is enhanced by using CpG and a Chk2 inhibitor drug in the transforming medium. Alternatively, human B cells can be activated by co-culture with transfected cell lines expressing CD40 Ligand (CD154) in medium containing additional soluble factors, such as IL-21 and human B cell Activating Factor (BAFF), a Type II member of the TNF superfamily. Fusion methods using Sendai virus or polyethylene glycol (PEG) are also known. The use of electrically induced fusion methods is also appropriate. Fusion procedures usually produce viable hybrids at low frequencies, about 1×10⁻⁶ to 1×10⁻⁸, but with optimized procedures one can achieve fusion efficiencies close to 1 in 200. However, relatively low efficiency of fusion does not pose a problem, as the viable, fused hybrids are differentiated from the parental, infused cells (particularly the infused myeloma cells that would normally continue to divide indefinitely) by culturing in a selective medium. The selective medium is generally one that contains an agent that blocks the de novo synthesis of nucleotides in the tissue culture medium. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the medium is supplemented with hypoxanthine and thymidine as a source of nucleotides (HAT medium). Where azaserine is used, the medium is supplemented with hypoxanthine. Ouabain is added if the B cell source is an EBV-transformed human B cell line, in order to eliminate EBV-transformed lines that have not fused to the myeloma.

The preferred selection medium is HAT or HAT with ouabain. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells. When the source of B cells used for fusion is a line of EBV-transformed B cells, as here, ouabain may also be used for drug selection of hybrids as EBV-transformed B cells are susceptible to drug killing, whereas the myeloma partner used is chosen to be ouabain resistant.

Culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays dot immunobinding assays, and the like. The selected hybridomas are then serially diluted or single-cell sorted by flow cytometric sorting and cloned into individual antibody-producing cell lines, which clones can then be propagated indefinitely to provide monoclonal antibodies. The cell lines may be exploited for monoclonal antibody production in two basic ways. A sample of the hybridoma can be injected (often into the peritoneal cavity) into an animal (e.g., a mouse). Optionally, the animals are primed with a hydrocarbon, especially oils such as pristane (tetramethylpentadecane) prior to injection. When human hybridomas are used in this way, it is optimal to inject immunocompromised mice, such as SCID mice, to prevent tumor rejection. The injected animal develops tumors secreting the specific monoclonal antibody produced by the fused cell hybrid. The body fluids of the animal, such as serum or ascites fluid, can then be tapped to provide monoclonal antibodies in high concentration. The individual cell lines could also be cultured in vitro, where the monoclonal antibodies are naturally secreted into the culture medium from which they can be readily obtained in high concentrations. Alternatively, human hybridoma cells lines can be used in vitro to produce immunoglobulins in cell supernatant. The cell lines can be adapted for growth in serum-free medium to optimize the ability to recover human monoclonal immunoglobulins of high purity.

Hybridomas may be cultured, then cells lysed, and total RNA extracted. Random hexamers may be used with RT to generate cDNA copies of RNA, and then PCR performed using a multiplex mixture of PCR primers expected to amplify all human variable gene sequences. PCR product can be cloned into pGEM-T Easy vector, then sequenced by automated DNA sequencing using standard vector primers. Assay of binding and neutralization may be performed using antibodies collected from hybridoma supernatants and purified by FPLC, using Protein G columns.

Recombinant full-length IgG antibodies can be generated by subcloning heavy and light chain Fv DNAs from the cloning vector into an IgG plasmid vector, transfected into 293 (e.g., Freestyle) cells or CHO cells, and antibodies can be collected and purified from the 293 or CHO cell supernatant. Other appropriate host cells systems include bacteria, such as E. coli, insect cells (S2, Sf9, Sf29, High Five), plant cells (e.g., tobacco, with or without engineering for human-like glycans), algae, or in a variety of non-human transgenic contexts, such as mice, rats, goats or cows.

Expression of nucleic acids encoding antibodies, both for the purpose of subsequent antibody purification, and for immunization of a host, is also contemplated. Antibody coding sequences can be RNA, such as native RNA or modified RNA. Modified RNA contemplates certain chemical modifications that confer increased stability and low immunogenicity to mRNAs, thereby facilitating expression of therapeutically important proteins. For instance, N1-methyl-pseudouridine (N1mΨ) outperforms several other nucleoside modifications and their combinations in terms of translation capacity. In addition to turning off the immune/eIF2α phosphorylation-dependent inhibition of translation, incorporated N1mΨ nucleotides dramatically alter the dynamics of the translation process by increasing ribosome pausing and density on the mRNA. Increased ribosome loading of modified mRNAs renders them more permissive for initiation by favoring either ribosome recycling on the same mRNA or de novo ribosome recruitment. Such modifications could be used to enhance antibody expression in vivo following inoculation with RNA. The RNA, whether native or modified, may be delivered as naked RNA or in a delivery vehicle, such as a lipid nanoparticle.

Alternatively, DNA encoding the antibody may be employed for the same purposes. The DNA is included in an expression cassette comprising a promoter active in the host cell for which it is designed. The expression cassette is advantageously included in a replicable vector, such as a conventional plasmid or minivector. Vectors include viral vectors, such as poxviruses, adenoviruses, herpesviruses, adeno-associated viruses, and lentiviruses are contemplated. Replicons encoding antibody genes such as alphavirus replicons based on VEE virus or Sindbis virus are also contemplated. Delivery of such vectors can be performed by needle through intramuscular, subcutaneous, or intradermal routes, or by transcutaneous electroporation when in vivo expression is desired.

Alternatively, a molecular cloning approach may be used to generate monoclonal antibodies. Single B cells labeled with the antigen of interest can be sorted physically using paramagnetic bead selection or flow cytometric sorting, then RNA can be isolated from the single cells and antibody genes amplified by RT-PCR. Alternatively, antigen-specific bulk sorted populations of cells can be segregated into microvesicles and the matched heavy and light chain variable genes recovered from single cells using physical linkage of heavy and light chain amplicons, or common barcoding of heavy and light chain genes from a vesicle. Matched heavy and light chain genes form single cells also can be obtained from populations of antigen specific B cells by treating cells with cell-penetrating nanoparticles bearing RT-PCR primers and barcodes for marking transcripts with one barcode per cell. The antibody variable genes also can be isolated by RNA extraction of a hybridoma line and the antibody genes obtained by RT-PCR and cloned into an immunoglobulin expression vector. Alternatively, combinatorial immunoglobulin phagemid libraries are prepared from RNA isolated from the cell lines and phagemids expressing appropriate antibodies are selected by panning using viral antigens. The advantages of this approach over conventional hybridoma techniques are that approximately 10⁴ times as many antibodies can be produced and screened in a single round, and that new specificities are generated by H and L chain combination which further increases the chance of finding appropriate antibodies.

Other U.S. patents, each incorporated herein by reference, that teach the production of antibodies useful in the present disclosure include U.S. Pat. No. 5,565,332, which describes the production of chimeric antibodies using a combinatorial approach; U.S. Pat. No. 4,816,567 which describes recombinant immunoglobulin preparations; and U.S. Pat. No. 4,867,973 which describes antibody-therapeutic agent conjugates.

Monoclonal antibodies produced by any means may be purified, if desired, using filtration, centrifugation, and various chromatographic methods, such as FPLC or affinity chromatography. Fragments of the monoclonal antibodies of the disclosure can be obtained from the purified monoclonal antibodies by methods that include digestion with enzymes, such as pepsin or papain, and/or by cleavage of disulfide bonds by chemical reduction. Alternatively, monoclonal antibody fragments encompassed by the present disclosure can be synthesized using an automated peptide synthesizer.

The antibodies of the present disclosure may be purified. The term “purified,” as used herein, is intended to refer to a composition, isolatable from other components, wherein the protein is purified to any degree relative to its naturally obtainable state. A purified protein therefore also refers to a protein, free from the environment in which it may naturally occur. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. Other methods for protein purification include, precipitation with ammonium sulfate, PEG, antibodies and the like or by heat denaturation, followed by centrifugation; gel filtration, reverse phase, hydroxylapatite and affinity chromatography; and combinations of such and other techniques.

In purifying an antibody of the present disclosure, it may be desirable to express the polypeptide in a prokaryotic or eukaryotic expression system and extract the protein using denaturing conditions. The polypeptide may be purified from other cellular components using an affinity column, which binds to a tagged portion of the polypeptide. As is generally known in the art, it is believed that the order of conducting the various purification steps may be changed, or that certain steps may be omitted, and still result in a suitable method for the preparation of a substantially purified protein or peptide.

Commonly, complete antibodies are fractionated utilizing agents (i.e., protein A) that bind the Fc portion of the antibody. Alternatively, antigens may be used to simultaneously purify and select appropriate antibodies. Such methods often utilize the selection agent bound to a support, such as a column, filter or bead. The antibodies are bound to a support, contaminants removed (e.g., washed away), and the antibodies released by applying conditions (salt, heat, etc.).

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. Another method for assessing the purity of a fraction is to calculate the specific activity of the fraction, to compare it to the specific activity of the initial extract, and to thus calculate the degree of purity. The actual units used to represent the amount of activity will, of course, be dependent upon the particular assay technique chosen to follow the purification and whether or not the expressed protein or peptide exhibits a detectable activity.

It is known that the migration of a polypeptide can vary, sometimes significantly, with different conditions of SDS/PAGE. It will therefore be appreciated that under differing electrophoresis conditions, the apparent molecular weights of purified or partially purified expression products may vary.

### J. Modification of Antibodies

The sequences of antibodies may be modified for a variety of reasons, such as improved expression, improved cross-reactivity, or diminished off-target binding. Modified antibodies may be made by any technique known to those of skill in the art, including expression through standard molecular biological techniques, or the chemical synthesis of polypeptides.

For example, one may wish to make modifications, such as introducing conservative changes into an antibody molecule. In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

The substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: basic amino acids: arginine (+3.0), lysine (+3.0), and histidine (−0.5); acidic amino acids: aspartate (+3.0±1), glutamate (+3.0±1), asparagine (+0.2), and glutamine (+0.2); hydrophilic, nonionic amino acids: serine (+0.3), asparagine (+0.2), glutamine (+0.2), and threonine (−0.4), sulfur containing amino acids: cysteine (−1.0) and methionine (−1.3); hydrophobic, nonaromatic amino acids: valine (−1.5), leucine (−1.8), isoleucine (−1.8), proline (−0.5±1), alanine (−0.5), and glycine (0); hydrophobic, aromatic amino acids: tryptophan (−3.4), phenylalanine (−2.5), and tyrosine (−2.3).

An amino acid can be substituted for another having a similar hydrophilicity and produce a biologically or immunologically modified protein. In such changes, the substitution of amino acids whose hydrophilicity values are within ±2 is preferred, those that are within ±1 are particularly preferred, and those within ±0.5 are even more particularly preferred.

Amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

The present disclosure also contemplates isotype modification. By modifying the Fc region to have a different isotype, different functionalities can be achieved. For example, changing to IgG₁ can increase antibody dependent cell cytotoxicity, switching to class A can improve tissue distribution, and switching to class M can improve valency.

One can design an Fc region of an antibody with altered effector function, e.g., by modifying C1q binding and/or FcγR binding and thereby changing CDC activity and/or ADCC activity. “Effector functions” are responsible for activating or diminishing a biological activity (e.g., in a subject). Examples of effector functions include, but are not limited to: C1q binding; complement dependent cytotoxicity (CDC); Fc receptor binding; antibody-dependent cell-mediated cytotoxicity (ADCC); phagocytosis; down regulation of cell surface receptors (e.g., B cell receptor; BCR), etc. Such effector functions may require the Fc region to be combined with a binding domain (e.g., an antibody variable domain) and can be assessed using various assays (e.g., Fc binding assays, ADCC assays, CDC assays, etc.).

For example, one can generate a variant Fc region of an antibody with improved C1q binding and improved FcγRIII binding (e.g., having both improved ADCC activity and improved CDC activity). Alternatively, if it is desired that effector function be reduced or ablated, a variant Fc region can be engineered with reduced CDC activity and/or reduced ADCC activity. In other embodiments, only one of these activities may be increased, and, optionally, also the other activity reduced (e.g., to generate an Fc region variant with improved ADCC activity, but reduced CDC activity and vice versa).

An isolated monoclonal antibody, or antigen binding fragment thereof, may contain a substantially homogeneous glycan without sialic acid, galactose, or fucose. The aforementioned substantially homogeneous glycan may be covalently attached to the heavy chain constant region.

A monoclonal antibody may have a novel Fc glycosylation pattern. Glycosylation of an Fc region is typically either N-linked or O-linked. N-linked refers to the attachment of the carbohydrate moiety to the side chain of an asparagine residue. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used. The recognition sequences for enzymatic attachment of the carbohydrate moiety to the asparagine side chain peptide sequences are asparagine-X-serine and asparagine-X-threonine, where X is any amino acid except proline. Thus, the presence of either of these peptide sequences in a polypeptide creates a potential glycosylation site.

The glycosylation pattern may be altered, for example, by deleting one or more glycosylation site(s) found in the polypeptide, and/or adding one or more glycosylation site(s) that are not present in the polypeptide. Addition of glycosylation sites to the Fc region of an antibody is conveniently accomplished by altering the amino acid sequence such that it contains one or more of the above-described tripeptide sequences (for N-linked glycosylation sites). An exemplary glycosylation variant has an amino acid substitution of residue Asn 297 of the heavy chain. The alteration may also be made by the addition of, or substitution by, one or more serine or threonine residues to the sequence of the original polypeptide (for O-linked glycosylation sites). Additionally, a change of Asn 297 to Ala can remove one of the glycosylation sites.

The isolated monoclonal antibody, or antigen binding fragment thereof, may be present in a substantially homogenous composition represented by the GNGN or G1/G2 glycoform, which exhibits increased binding affinity for Fc gamma RI and Fc gamma RIII compared to the same antibody without the substantially homogeneous GNGN glycoform and with G0, G1F, G2F, GNF, GNGNF or GNGNFX containing glycoforms. Fc glycosylation plays a significant role in anti-viral and anti-cancer properties of therapeutic mAbs. Elimination of core fucose dramatically improves the ADCC activity of mAbs mediated by natural killer (NK) cells but appears to have the opposite effect on the ADCC activity of polymorphonuclear cells (PMNs).

The isolated monoclonal antibody, or antigen binding fragment thereof, may be expressed in cells that express beta (1,4)-N-acetylglucosaminyltransferase III (GnT III), such that GnT III adds GlcNAc to the antibody. Methods for producing antibodies in such a fashion are provided in WO/9954342 and WO/03011878. Cell lines can be altered to enhance or reduce or eliminate certain post-translational modifications, such as glycosylation, using genome editing technology such as Clustered Regularly Interspaced Short Palindromic Repeats (CRISPR). For example, CRISPR technology can be used to eliminate genes encoding glycosylating enzymes in 293 or CHO cells used to express monoclonal antibodies.

It is possible to engineer the antibody variable gene sequences obtained from human B cells to enhance their manufacturability and safety. Potential protein sequence liabilities can be identified by searching for sequence motifs associated with sites containing:
1) Unpaired Cys residues,
2) N-linked glycosylation,
3) Asn deamidation,
4) Asp isomerization,
5) SYE truncation,
6) Met oxidation,
7) Trp oxidation,
8) N-terminal glutamate,
9) Integrin binding,
10) CD11c/CD18 binding, or
11) Fragmentation
   Such motifs can be eliminated by altering the synthetic gene comprising the cDNA encoding the antibodies.

Antibodies can be engineered to enhance solubility. For example, some hydrophilic residues such as aspartic acid, glutamic acid, and serine contribute significantly more favorably to protein solubility than other hydrophilic residues, such as asparagine, glutamine, threonine, lysine, and arginine.

B cell repertoire deep sequencing of human B cells from blood donors has been performed on a wide scale. Sequence information about a significant portion of the human antibody repertoire facilitates statistical assessment of antibody sequence features common in healthy humans. With knowledge about the antibody sequence features in a human recombined antibody variable gene reference database, the position specific degree of “Human Likeness” (HL) of an antibody sequence can be estimated. HL has been shown to be useful for the development of antibodies in clinical use, like therapeutic antibodies or antibodies as vaccines. The goal is to increase the human likeness of antibodies to reduce potential adverse effects and anti-antibody immune responses that will lead to significantly decreased efficacy of the antibody drug or can induce serious health implications. One can assess antibody characteristics of the combined antibody repertoire of three healthy human blood donors of about 400 million sequences in total and created a novel “relative Human Likeness” (rHL) score that focuses on the hypervariable region of the antibody. The rHL score allows one to easily distinguish between human (positive score) and non-human sequences (negative score). Antibodies can be engineered to eliminate residues that are not common in human repertoires.

### K. Characterization of Antibodies

Antibodies according to the present disclosure may be defined, in the first instance, by their binding specificity. Those of skill in the art, by assessing the binding specificity/affinity of a given antibody using techniques well known to those of skill in the art, can determine whether such antibodies fall within the scope of the instant claims. For example, the epitope to which a given antibody binds may consist of a single contiguous sequence of 3 or more (e.g. 3, 4, 5, 6, 7, 8, 9, 10, 11, 12, 13, 14, 15, 16, 17, 18, 19, 20) amino acids located within the antigen molecule (e.g., a linear epitope in a domain). Alternatively, the epitope may consist of a plurality of non-contiguous amino acids (or amino acid sequences) located within the antigen molecule (e.g., a conformational epitope).

Various techniques known to persons of ordinary skill in the art can be used to determine whether an antibody “interacts with one or more amino acids” within a polypeptide or protein. Exemplary techniques include, for example, routine cross-blocking assays, such as that described in Antibodies, Harlow and Lane (Cold Spring Harbor Press, Cold Spring Harbor, N.Y.). Cross-blocking can be measured in various binding assays such as ELISA, biolayer interferometry, or surface plasmon resonance. Other methods include alanine scanning mutational analysis, peptide blot analysis (Reineke (2004) Methods Mol. Biol. 248: 443-63), peptide cleavage analysis, high-resolution electron microscopy techniques using single particle reconstruction, cryoEM, or tomography, crystallographic studies and NMR analysis. In addition, methods such as epitope excision, epitope extraction and chemical modification of antigens can be employed (Tomer (2000) Prot. Sci. 9: 487-496). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water and exchangeable protons within amino acids that are protected by the antibody complex undergo deuterium-to-hydrogen back-exchange at a slower rate than exchangeable protons within amino acids that are not part of the interface. As a result, amino acids that form part of the protein/antibody interface may retain deuterium and therefore exhibit relatively higher mass compared to amino acids not included in the interface. After dissociation of the antibody, the target protein is subjected to protease cleavage and mass spectrometry analysis, thereby revealing the deuterium-labeled residues which correspond to the specific amino acids with which the antibody interacts. See, e.g., Ehring (1999) Analytical Biochemistry 267: 252-259; Engen and Smith (2001) Anal. Chem. 73: 256A-265A.

The term “epitope” refers to a site on an antigen to which B and/or T cells respond. B-cell epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5 or 8-10 amino acids in a unique spatial conformation.

Modification-Assisted Profiling (MAP), also known as Antigen Structure-based Antibody Profiling (ASAP) is a method that categorizes large numbers of monoclonal antibodies directed against the same antigen according to the similarities of the binding profile of each antibody to chemically or enzymatically modified antigen surfaces (see US 2004/0101920, herein specifically incorporated by reference in its entirety). Each category may reflect a unique epitope either distinctly different from or partially overlapping with epitope represented by another category. This technology allows rapid filtering of genetically identical antibodies, such that characterization can be focused on genetically distinct antibodies. When applied to hybridoma screening, MAP may facilitate identification of rare hybridoma clones that produce monoclonal antibodies having the desired characteristics. MAP may be used to sort the antibodies of the disclosure into groups of antibodies binding different epitopes.

The present disclosure includes antibodies that may bind to the same epitope, or a portion of the same epitope. One can easily determine whether an antibody binds to the same epitope as, or competes for binding with, a reference antibody by using routine methods known in the art. For example, to determine if a test antibody binds to the same epitope as a reference antibody, the reference antibody is allowed to bind to the target molecule under saturating conditions. Next, the ability of a test antibody to bind to the target molecule is assessed. If the test antibody is able to bind to the target molecule following saturation binding with the reference antibody, it can be concluded that the test antibody binds to a different epitope than the reference antibody. On the other hand, if the test antibody is not able to bind to the target molecule following saturation binding with the reference antibody, then the test antibody may bind to the same epitope as the epitope bound by the reference antibody.

To determine if a test antibody competes for binding with a disclosed antibody, the above-described binding methodology is performed in two orientations: In a first orientation, the disclosed antibody is allowed to bind to an coronavirus spike protein under saturating conditions followed by assessment of binding of the test antibody to the coronavirus spike protein. In a second orientation, the test antibody is allowed to bind to a coronavirus spike protein under saturating conditions followed by assessment of binding of the disclosed antibody to the coronavirus spike protein. If, in both orientations, only the first (saturating) antibody is capable of binding to the coronavirus spike protein, then it is concluded that the test antibody and the disclosed antibody compete for binding to the coronavirus spike protein. As will be appreciated by a person of ordinary skill in the art, a test antibody that competes for binding with a disclosed antibody may not necessarily bind to the identical epitope as the disclosed antibody, but may sterically block binding of the disclosed antibody by binding an overlapping or adjacent epitope.

Two antibodies bind to the same or overlapping epitope if each competitively inhibits (blocks) binding of the other to the antigen. That is, a 1-, 5-, 10-, 20- or 100-fold excess of one antibody inhibits binding of the other by at least 50% but preferably 75%, 90%, or even 99% as measured in a competitive binding assay (see, e.g., Junghans et al., Cancer Res. 1990 50:1495-1502). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, surface plasmon resonance, flow cytometry or any other quantitative or qualitative antibody-binding assay available in the art.

In another aspect, the antibodies may be defined by their variable sequence, which include additional “framework” regions. These are provided in Table 4 that represent full variable regions. Furthermore, the antibodies sequences may vary from these sequences, optionally using methods discussed in greater detail below. For example, nucleic acid sequences may vary from those set out above in that (a) the variable regions may be segregated away from the constant domains of the light and heavy chains, (b) the nucleic acids may vary from those set out above while not affecting the residues encoded thereby, (c) the nucleic acids may vary from those set out above by a given percentage, e.g., 70%, 75%, 80%, 85%, 90%, 91%, 92%, 93%, 94%, 95%, 96%, 97%, 98% or 99% homology, (d) the nucleic acids may vary from those set out above by virtue of the ability to hybridize under high stringency conditions, as exemplified by low salt and/or high temperature conditions, such as provided by about 0.02 M to about 0.15 M NaCl at temperatures of about 50° C. to about 70° C., (e) the amino acids may vary from those set out above by a given percentage, e.g., 80%, 85%, 90%, 91%, 92%, 93%, 94%, 95%, 96%, 97%, 98% or 99% homology, or (f) the amino acids may vary from those set out above by permitting conservative substitutions. Each of the foregoing applies to the amino acid sequences of Tables 4 and 5.

When comparing polynucleotide and polypeptide sequences, two sequences are said to be “identical” if the sequence of nucleotides or amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One particular example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides and polypeptides of the disclosure. Software for performing BLAST analyses is publicly available through the National Center for Biotechnology Information. The rearranged nature of an antibody sequence and the variable length of each gene requires multiple rounds of BLAST searches for a single antibody sequence. Also, manual assembly of different genes is difficult and error-prone. The sequence analysis tool IgBLAST (world-wide-web at ncbi.nlm.nih.gov/igblast/) identifies matches to the germline V, D and J genes, details at rearrangement junctions, the delineation of Ig V domain framework regions and complementarity determining regions. IgBLAST can analyze nucleotide or protein sequences and can process sequences in batches and allows searches against the germline gene databases and other sequence databases simultaneously to minimize the chance of missing possibly the best matching germline V gene.

By “germline nucleic acid residue” is meant the nucleic acid residue that naturally occurs in a germline gene encoding a constant or variable region. “Germline gene” is the DNA found in a germ cell (i.e., a cell destined to become an egg or in the sperm). A “germline mutation” refers to a heritable change in a particular DNA that has occurred in a germ cell or the zygote at the single-cell stage, and when transmitted to offspring, such a mutation is incorporated in every cell of the body. A germline mutation is in contrast to a somatic mutation which is acquired in a single body cell. In some cases, nucleotides in a germline DNA sequence encoding for a variable region are mutated (i.e., a somatic mutation) and replaced with a different nucleotide.

In one approach, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residues occur in both sequences to yield the number of matched positions, dividing the number of matched positions by the total number of positions in the reference sequence (i.e., the window size) and multiplying the results by 100 to yield the percentage of sequence identity.

Yet another way of defining an antibody is as a “derivative” of any of the antibodies provided herein and their antigen-binding fragments. A derivative antibody or antibody fragment may be modified by chemical modifications using techniques known to those of skill in the art, including, but not limited to, specific chemical cleavage, acetylation, formulation, metabolic synthesis of tunicamycin, etc. In one embodiment, an antibody derivative will possess a similar or identical function as the parental antibody. In another embodiment, an antibody derivative will exhibit an altered activity relative to the parental antibody. For example, a derivative antibody (or fragment thereof) can bind to its epitope more tightly or be more resistant to proteolysis than the parental antibody.

The term “derivative” refers to an antibody or antigen-binding fragment thereof that immunospecifically binds to an antigen but which comprises, one, two, three, four, five or more amino acid substitutions, additions, deletions or modifications relative to a “parental” (or wild-type) molecule. Such amino acid substitutions or additions may introduce naturally occurring (i.e., DNA-encoded) or non-naturally occurring amino acid residues. The term “derivative” encompasses, for example, as variants having altered CH1, hinge, CH2, CH3 or CH4 regions, so as to form, for example antibodies, etc., having variant Fc regions that exhibit enhanced or impaired effector or binding characteristics. The term “derivative” additionally encompasses non-amino acid modifications, for example, amino acids that may be glycosylated (e.g., have altered mannose, 2-N-acetylglucosamine, galactose, fucose, glucose, sialic acid, 5-N-acetylneuraminic acid, 5-glycolneuraminic acid, etc. content), acetylated, pegylated, phosphorylated, amidated, derivatized by known protecting/blocking groups, proteolytic cleavage, linked to a cellular ligand or other protein, etc. In some embodiments, the altered carbohydrate modifications modulate one or more of the following: solubilization of the antibody, facilitation of subcellular transport and secretion of the antibody, promotion of antibody assembly, conformational integrity, and antibody-mediated effector function. In a specific embodiment, the altered carbohydrate modifications enhance antibody mediated effector function relative to the antibody lacking the carbohydrate modification. Carbohydrate modifications that lead to altered antibody mediated effector function are well known in the art.

A derivative antibody or antibody fragment can be generated with an engineered sequence or glycosylation state to confer preferred levels of activity in antibody dependent cellular cytotoxicity (ADCC), antibody-dependent cellular phagocytosis (ADCP), antibody-dependent neutrophil phagocytosis (ADNP), or antibody-dependent complement deposition (ADCD) functions as measured by bead-based or cell-based assays or in vivo studies in animal models.

A derivative antibody or antibody fragment may be modified by chemical modifications using techniques known to those of skill in the art, including, but not limited to, specific chemical cleavage, acetylation, formulation, metabolic synthesis of tunicamycin, etc. In one embodiment, an antibody derivative will possess a similar or identical function as the parental antibody. In another embodiment, an antibody derivative will exhibit an altered activity relative to the parental antibody. For example, a derivative antibody (or fragment thereof) can bind to its epitope more tightly or be more resistant to proteolysis than the parental antibody.

One can determine the biophysical properties of antibodies. One can use elevated temperature to unfold antibodies to determine relative stability, using average apparent melting temperatures. Differential Scanning Calorimetry (DSC) measures the heat capacity, Cₚ, of a molecule (the heat required to warm it, per degree) as a function of temperature. One can use DSC to study the thermal stability of antibodies. DSC data for mAbs is particularly interesting because it sometimes resolves the unfolding of individual domains within the mAb structure, producing up to three peaks in the thermogram (from unfolding of the Fab, C_{H}2, and C_{H}3 domains). Typically unfolding of the Fab domain produces the strongest peak. The DSC profiles and relative stability of the Fc portion show characteristic differences for the human IgG₁, IgG₂, IgG₃, and IgG₄ subclasses (Garber and Demarest, Biochem. Biophys. Res. Commun. 355, 751-757, 2007). One also can determine average apparent melting temperature using circular dichroism (CD), performed with a CD spectrometer. Far-UV CD spectra will be measured for antibodies in the range of 200 to 260 nm at increments of 0.5 nm. The final spectra can be determined as averages of 20 accumulations. Residue ellipticity values can be calculated after background subtraction. Thermal unfolding of antibodies (0.1 mg/mL) can be monitored at 235 nm from 25-95° C. and a heating rate of 1° C./min. One can use dynamic light scattering (DLS) to assess for propensity for aggregation. DLS is used to characterize size of various particles including proteins. If the system is not disperse in size, the mean effective diameter of the particles can be determined. This measurement depends on the size of the particle core, the size of surface structures, and particle concentration. Since DLS essentially measures fluctuations in scattered light intensity due to particles, the diffusion coefficient of the particles can be determined. DLS software in commercial DLA instruments displays the particle population at different diameters. Stability studies can be done conveniently using DLS. DLS measurements of a sample can show whether the particles aggregate over time or with temperature variation by determining whether the hydrodynamic radius of the particle increases. If particles aggregate, one can see a larger population of particles with a larger radius. Stability depending on temperature can be analyzed by controlling the temperature in situ. Capillary electrophoresis (CE) techniques include proven methodologies for determining features of antibody stability. One can use an iCE approach to resolve antibody protein charge variants due to deamidation, C-terminal lysines, sialylation, oxidation, glycosylation, and any other change to the protein that can result in a change in pI of the protein. Each of the expressed antibody proteins can be evaluated by high throughput, free solution isoelectric focusing (IEF) in a capillary column (cIEF), using a Protein Simple Maurice instrument. Whole-column UV absorption detection can be performed every 30 seconds for real time monitoring of molecules focusing at the isoelectric points (pIs). This approach combines the high resolution of traditional gel IEF with the advantages of quantitation and automation found in column-based separations while eliminating the need for a mobilization step. The technique yields reproducible, quantitative analysis of identity, purity, and heterogeneity profiles for the expressed antibodies. The results identify charge heterogeneity and molecular sizing on the antibodies, with both absorbance and native fluorescence detection modes and with sensitivity of detection down to 0.7 μg/mL.

One can determine the intrinsic solubility score of antibody sequences. The intrinsic solubility scores can be calculated using CamSol Intrinsic (Sormanni et al., J Mol Biol 427, 478-490, 2015). The amino acid sequences for residues 95-102 (Kabat numbering) in HCDR3 of each antibody fragment such as a scFv can be evaluated via the online program to calculate the solubility scores. One also can determine solubility using laboratory techniques. Various techniques exist, including addition of lyophilized protein to a solution until the solution becomes saturated and the solubility limit is reached, or concentration by ultrafiltration in a microconcentrator with a suitable molecular weight cut-off. The most straightforward method is induction of amorphous precipitation, which measures protein solubility using a method involving protein precipitation using ammonium sulfate (Trevino et al., J Mol Biol, 366: 449-460, 2007). Ammonium sulfate precipitation gives quick and accurate information on relative solubility values. Ammonium sulfate precipitation produces precipitated solutions with well-defined aqueous and solid phases and requires relatively small amounts of protein. Solubility measurements performed using induction of amorphous precipitation by ammonium sulfate also can be done easily at different pH values. Protein solubility is highly pH dependent, and pH is considered the most important extrinsic factor that affects solubility.

Generally, it is thought that autoreactive clones should be eliminated during ontogeny by negative selection; however, it has become clear that many human naturally occurring antibodies with autoreactive properties persist in adult mature repertoires, and the autoreactivity may enhance the antiviral function of many antibodies to pathogens. It has been noted that HCDR3 loops in antibodies during early B cell development are often rich in positive charge and exhibit autoreactive patterns (Wardemann et al., Science 301, 1374-1377, 2003). One can test a given antibody for autoreactivity by assessing the level of binding to human origin cells in microscopy (using adherent HeLa or HEp-2 epithelial cells) and flow cytometric cell surface staining (using suspension Jurkat T cells and 293S human embryonic kidney cells). Autoreactivity also can be surveyed using assessment of binding to tissues in tissue arrays.

### IV. Chimeric Antigen Receptors

Chimeric antigen receptor (CAR) molecules are recombinant fusion protein and are distinguished by their ability to both bind antigen and transduce activation signals via immunoreceptor activation motifs (ITAMs) present in their cytoplasmic tails in order to activate genetically modified immune effector cells for killing, proliferation, and cytokine production. Receptor constructs utilizing an antigen-binding moiety (for example, generated from single chain antibodies (scFv)) afford the additional advantage of being “universal” in that they bind native antigen on the target cell surface in an HLA-independent fashion.

Embodiments of the CARs described herein include nucleic acids encoding an antigen-specific CAR polypeptide comprising an intracellular signaling domain, a transmembrane domain, and an extracellular domain comprising an antigen-binding domain. A CAR may recognize an epitope comprised of the shared space between one or more antigens. Optionally, a CAR can comprise a hinge domain positioned between the transmembrane domain and the antigen binding domain. A CAR may further comprise a signal peptide that directs expression of the CAR to the cell surface. For example, a CAR may comprise a signal peptide from GM-CSF. A CAR may also be co-expressed with a membrane-bound cytokine to improve persistence. For example, a CAR may be co-expressed with membrane-bound IL-15.

Depending on the arrangement of the domains of the CAR and the specific sequences used in the domains, immune effector cells expressing the CAR may have different levels activity against target cells. Different CAR sequences may be introduced into immune effector cells to generate engineered cells, the engineered cells selected for elevated SRC, and the selected cells tested for activity to identify the CAR constructs predicted to have the greatest therapeutic efficacy.

A chimeric antigen receptor can be produced by any means known in the art, though preferably it is produced using recombinant DNA techniques. A nucleic acid sequence encoding the several regions of the chimeric antigen receptor can be prepared and assembled into a complete coding sequence by standard techniques of molecular cloning (genomic library screening, PCR, primer-assisted ligation, scFv libraries from yeast and bacteria, site-directed mutagenesis, etc.). The resulting coding region can be inserted into an expression vector and used to transform a suitable expression host allogeneic or autologous immune effector cells, such as a T cell or an NK cell.

The chimeric construct may be introduced into immune effector cells as naked DNA or in a suitable vector. Methods of stably transfecting cells by electroporation using naked DNA are known in the art. See, e.g., U.S. Pat. No. 6,410,319. Naked DNA generally refers to the DNA encoding a chimeric receptor contained in a plasmid expression vector in proper orientation for expression. Alternatively, a viral vector (e.g., a retroviral vector, adenoviral vector, adeno-associated viral vector, or lentiviral vector) can be used to introduce the chimeric construct into immune effector cells. Suitable vectors for use in accordance with the method of the present invention are non-replicating in the immune effector cells. A large number of vectors are known that are based on viruses, where the copy number of the virus maintained in the cell is low enough to maintain the viability of the cell, such as, for example, vectors based on HIV, SV40, EBV, HSV, or BPV.

### A. Antigen Binding Domains

An antigen binding domain may comprise complementary determining regions of a monoclonal antibody, variable regions of a monoclonal antibody, and/or antigen binding fragments thereof. The antigen binding regions or domains may comprise a fragment of the VH and VL chains of a single-chain variable fragment (scFv) derived from a particular mouse, humanized, or human monoclonal antibody. The fragment can also be any number of different antigen binding domains of an antigen-specific antibody. The fragment may be an antigen-specific scFv encoded by a sequence that is optimized for human codon usage for expression in human cells. In certain aspects, VH and VL domains of a CAR are separated by a linker sequence, such as a Whitlow linker.

The prototypical CAR encodes a scFv comprising VH and VL domains derived from one monoclonal antibody (mAb), coupled to a transmembrane domain and one or more cytoplasmic signaling domains (e.g. costimulatory domains and signaling domains). Thus, a CAR may comprise the LCDR1-3 sequences and the HCDR1-3 sequences of an antibody that binds to coronavirus spike protein. In further aspects, however, two of more antibodies that bind to an antigen of interest are identified and a CAR is constructed that comprises: (1) the HCDR1-3 sequences of a first antibody that binds to the antigen; and (2) the LCDR1-3 sequences of a second antibody that binds to the antigen. Such a CAR that comprises HCDR and LCDR sequences from two different antigen binding antibodies may have the advantage of preferential binding to particular conformations of an antigen (e.g., conformations preferentially associated with cancer cells versus normal tissue).

Alternatively, a CAR may be engineered using VH and VL chains derived from different mAbs to generate a panel of CAR+ immune effector cells. The antigen binding domain of a CAR may contain any combination of the LCDR1-3 sequences of a first antibody and the HCDR1-3 sequences of a second antibody.

### B. Hinge Domains

A CAR polypeptide may include a hinge domain positioned between the antigen binding domain and the transmembrane domain. In some cases, a hinge domain may be included in CAR polypeptides to provide adequate distance between the antigen binding domain and the cell surface or to alleviate possible steric hindrance that could adversely affect antigen binding or effector function of CAR-modified immune effector cells. The hinge domain may comprise a sequence that binds to an Fc receptor, such as FcγR2a or FcγR1a. For example, the hinge sequence may comprise an Fc domain from a human immunoglobulin (e.g., IgG1, IgG2, IgG3, IgG4, IgA1, IgA2, IgM, IgD or IgE) that binds to an Fc receptor.

A CAR hinge domain may be derived from human immunoglobulin (Ig) constant region or a portion thereof including the Ig hinge, or from human CD8 α transmembrane domain and CD8a-hinge region. A CAR hinge domain may comprise a hinge-CH2-CH3 region of antibody isotype IgG4. The hinge domain (and/or the CAR) may not comprise a wild type human IgG4 CH2 and CH3 sequence. Point mutations may be introduced in antibody heavy chain CH₂ domain to reduce glycosylation and non-specific Fc gamma receptor binding of CAR-modified immune effector cells.

A CAR hinge domain may comprise an Ig Fc domain that comprises at least one mutation relative to wild type Ig Fc domain that reduces Fc-receptor binding. For example, the CAR hinge domain can comprise an IgG4-Fc domain that comprises at least one mutation relative to wild type IgG4-Fc domain that reduces Fc-receptor binding. A CAR hinge domain may comprise an IgG4-Fc domain having a mutation (such as an amino acid deletion or substitution) at a position corresponding to L235 and/or N297 relative to the wild type IgG4-Fc sequence. For example, a CAR hinge domain can comprise an IgG4-Fc domain having a L235E and/or a N297Q mutation relative to the wild type IgG4-Fc sequence. A CAR hinge domain may comprise an IgG4-Fc domain having an amino acid substitution at position L235 for an amino acid that is hydrophilic, such as R, H, K, D, E, S, T, N or Q, or that has similar properties to an “E,” such as D. A CAR hinge domain may comprise an IgG4-Fc domain having an amino acid substitution at position N297 for an amino acid that has similar properties to a “Q,” such as S or T.

The hinge domain may comprise a sequence that is about 85%, 90%, 91%, 92%, 93%, 94%, 95%, 96%, 97%, 98%, 99% or 100% identical to an IgG4 hinge domain, a CD8a hinge domain, a CD28 hinge domain, or an engineered hinge domain.

### C. Transmembrane Domains

The antigen-specific extracellular domain and the intracellular signaling-domain may be linked by a transmembrane domain. Polypeptide sequences that can be used as part of transmembrane domain include, without limitation, the human CD4 transmembrane domain, the human CD28 transmembrane domain, the transmembrane human CD3Q domain, a cysteine mutated human CD3Q domain, or other transmembrane domains from other human transmembrane signaling proteins, such as CD16, CD8, and erythropoietin receptor. For example, the transmembrane domain may comprise a sequence at least 85%, 90%, 91%, 92%, 93%, 94%, 95%, 96%, 97%, 98%, 99% or 100% identical to one of those provided in U.S. Patent Publication No. 2014/0274909 (e.g. a CD8 and/or a CD28 transmembrane domain) or U.S. Pat. No. 8,906,682 (e.g. a CD8u transmembrane domain), both incorporated herein by reference. Transmembrane regions may be derived from (i.e. comprise at least the transmembrane region(s) of) the alpha, beta or zeta chain of the T-cell receptor, CD3 epsilon, CD45, CD4, CD5, CD8, CD9, CD16, CD22, CD33, CD37, CD64, CD80, CD86, CD134, CD137, CD154. In certain specific aspects, the transmembrane domain can be 85%, 90%, 91%, 92%, 93%, 94%, 95%, 96%, 97%, 98%, 99% or 100% identical to a CD8a transmembrane domain or a CD28 transmembrane domain.

### D. Intracellular Signaling Domains

The intracellular signaling domain of a CAR is responsible for activation of at least one of the normal effector functions of the immune cell engineered to express the CAR. The term “effector function” refers to a specialized function of a differentiated cell. Effector function of a T cell, for example, may be cytolytic activity or helper activity including the secretion of cytokines. Effector function in a naive, memory, or memory-type T cell includes antigen-dependent proliferation. Thus the term “intracellular signaling domain” refers to the portion of a protein that transduces the effector function signal and directs the cell to perform a specialized function. The intracellular signaling domain may be derived from the intracellular signaling domain of a native receptor. Examples of such native receptors include the zeta chain of the T-cell receptor or any of its homologs (e.g., eta, delta, gamma, or epsilon), MB1 chain, B29, Fc RIII, Fc RI, and combinations of signaling molecules, such as CD3ζ and CD28, CD27, 4-1BB/CD137, ICOS/CD278, IL-2Rβ/CD122, IL-2Rα/CD132, DAP10, DAP12, CD40, OX40/CD134, and combinations thereof, as well as other similar molecules and fragments. Intracellular signaling portions of other members of the families of activating proteins can be used.

While the entire intracellular signaling domain may be employed, in many cases it will not be necessary to use the entire intracellular polypeptide. To the extent that a truncated portion of the intracellular signaling domain may find use, such truncated portion may be used in place of the intact chain as long as it still transduces the effector function signal. The term “intracellular signaling domain” is thus meant to include a truncated portion of the intracellular signaling domain sufficient to transduce the effector function signal, upon CAR binding to a target. One or multiple cytoplasmic domains may be employed, as so-called third generation CARs have at least two or three signaling domains fused together for additive or synergistic effect, for example the CD28 and 4-1BB can be combined in a CAR construct. In certain specific aspects, the intracellular signaling domain comprises a sequence 85%, 90%, 91%, 92%, 93%, 94%, 95%, 96%, 97%, 98%, 99% or 100% identical to a CD3Q intracellular domain, a CD28 intracellular domain, a CD137 intracellular domain, or a domain comprising a CD28 intracellular domain fused to the 4-1BB intracellular domain.

### E. Immune Effector Cells

Immune effectors cells may be T cells (e.g., regulatory T cells, CD4+ T cells, CD8+ T cells, or gamma-delta T cells), natural killer (NK) cells, invariant NK cells, or NKT cells. Also provided herein are methods of producing and engineering the immune effector cells as well as methods of using and administering the cells for adoptive cell therapy, in which case the cells may be autologous or allogeneic. Thus, the immune effector cells may be used as immunotherapy, such as to target cancer cells.

The immune effector cells may be isolated from subjects, particularly human subjects. The immune effector cells can be obtained from a subject of interest, such as a subject suspected of having a particular disease or condition, a subject suspected of having a predisposition to a particular disease or condition, a subject who is undergoing therapy for a particular disease or condition, a subject who is a healthy volunteer or healthy donor, or from a blood bank. Immune effector cells can be collected, enriched, and/or purified from any tissue or organ in which they reside in the subject including, but not limited to, blood, cord blood, spleen, thymus, lymph nodes, bone marrow, tissues removed and/or exposed during surgical procedures, and tissues obtained via biopsy procedures. The isolated immune effector cells may be used directly, or they can be stored for a period of time, such as by freezing.

Tissues/organs from which the immune effector cells are enriched, isolated, and/or purified may be isolated from both living and non-living subjects, wherein the non-living subjects are organ donors. Immune effector cells isolated from cord blood may have enhanced immunomodulation capacity, such as measured by CD4- or CD8-positive T cell suppression. The immune effector cells may be isolated from pooled blood, particularly pooled cord blood, for enhanced immunomodulation capacity. The pooled blood may be from 2 or more sources, such as 3, 4, 5, 6, 7, 8, 9, 10 or more sources (e.g., donor subjects).

The population of immune cells can be obtained from a subject in need of therapy or suffering from a disease associated with reduced immune effector cell activity. Thus, the cells will be autologous to the subject in need of therapy. Alternatively, the population of immune effector cells can be obtained from a donor, preferably an allogeneic donor. Allogeneic donor cells may or may not be human-leukocyte-antigen (HLA)-compatible. To be rendered subject-compatible, allogeneic cells can be treated to reduce immunogenicity.

### 2. T Cells

The immune effector cells may be T cells. The T cells may be derived from the blood, bone marrow, lymph, umbilical cord, or lymphoid organs. The T cells may be human T cells. The T cells typically are primary cells, such as those isolated directly from a subject and/or isolated from a subject and frozen. The cells may include one or more subsets of T cells or other cell types, such as whole T cell populations, CD4⁺ cells, CD8⁺ cells, and subpopulations thereof, such as those defined by function, activation state, maturity, potential for differentiation, expansion, recirculation, localization, persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. For off-the-shelf technologies, the cells may be derived from pluripotent and/or multipotent cells, such as stem cells, such as induced pluripotent stem cells (iPSCs).

Among the sub-types and subpopulations of T cells (e.g., CD4⁺ and/or CD8⁺ T cells) are naive T (T_{N}) cells, effector T cells (T_{EFF}), memory T cells and sub-types thereof, such as stem cell memory T (TSC_{M}), central memory T (TC_{M}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

One or more of the T cell populations may be enriched for or depleted of cells that are positive for a specific marker, such as surface markers, or that are negative for a specific marker. In some cases, such markers are those that are absent or expressed at relatively low levels on certain populations of T cells (e.g., non-memory cells) but are present or expressed at relatively higher levels on certain other populations of T cells (e.g., memory cells).

T cells may be separated from a PBMC sample by negative selection of markers expressed on non-T cells, such as B cells, monocytes, or other white blood cells, such as CD14. In some aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations.

CD8⁺ T cells may be further enriched for or depleted of naive, central memory, effector memory, and/or central memory stem cells, such as by positive or negative selection based on surface antigens associated with the respective subpopulation. Enrichment for central memory T (T_{CM}) cells may be carried out to increase efficacy, such as to improve long-term survival, expansion, and/or engraftment following administration, which in some aspects is particularly robust in such sub-populations.

The T cells may be autologous T cells. In this method, tumor samples are obtained from patients and a single cell suspension is obtained. The single cell suspension can be obtained in any suitable manner, e.g., mechanically (disaggregating the tumor using, e.g., a gentleMACS™ Dissociator, Miltenyi Biotec, Auburn, Calif.) or enzymatically (e.g., collagenase or DNase). Single-cell suspensions of tumor enzymatic digests are cultured in interleukin-2 (IL-2). The cells are cultured until confluence (e.g., about 2×10⁶ lymphocytes), e.g., from about 5 to about 21 days, preferably from about 10 to about 14 days.

The cultured T cells can be pooled and rapidly expanded. Rapid expansion provides an increase in the number of antigen-specific T cells of at least about 50-fold (e.g., 50-, 60-, 70-, 80-, 90-, or 100-fold, or greater) over a period of about 10 to about 14 days. More preferably, rapid expansion provides an increase of at least about 200-fold (e.g., 200-, 300-, 400-, 500-, 600-, 700-, 800-, 900-, or greater) over a period of about 10 to about 14 days.

Expansion can be accomplished by any of a number of methods as are known in the art. For example, T cells can be rapidly expanded using non-specific T-cell receptor stimulation in the presence of feeder lymphocytes and either interleukin-2 (IL-2) or interleukin-15 (IL-15), with IL-2 being preferred. The non-specific T-cell receptor stimulus can include around 30 ng/ml of OKT3, a mouse monoclonal anti-CD3 antibody (available from Ortho-McNeil®, Raritan, N.J.). Alternatively, T cells can be rapidly expanded by stimulation of peripheral blood mononuclear cells (PBMC) in vitro with one or more antigens (including antigenic portions thereof, such as epitope(s), or a cell) of the cancer, which can be optionally expressed from a vector, such as a human leukocyte antigen A2 (HLA-A2) binding peptide, in the presence of a T-cell growth factor, such as 300 IU/ml IL-2 or IL-15, with IL-2 being preferred. The in vitro-induced T-cells are rapidly expanded by re-stimulation with the same antigen(s) of the cancer pulsed onto HLA-A2-expressing antigen-presenting cells. Alternatively, the T-cells can be re-stimulated with irradiated, autologous lymphocytes or with irradiated HLA-A2+ allogeneic lymphocytes and IL-2, for example.

The autologous T-cells can be modified to express a T-cell growth factor that promotes the growth and activation of the autologous T-cells. Suitable T-cell growth factors include, for example, interleukin (IL)-2, IL-7, IL-15, and IL-12. Suitable methods of modification are known in the art. See, for instance, Sambrook et al., MOLECULAR CLONING: A LABORATORY MANUAL, 3^{rd} ed., Cold Spring Harbor Press, Cold Spring Harbor, N.Y. 2001; and Ausubel et al., CURRENT PROTOCOLS IN MOLECULAR BIOLOGY, Greene Publishing Associates and John Wiley & Sons, N Y, 1994. In particular aspects, modified autologous T-cells express the T-cell growth factor at high levels. T-cell growth factor coding sequences, such as that of IL-12, are readily available in the art, as are promoters, the operable linkage of which to a T-cell growth factor coding sequence promote high-level expression.

### 3. NK Cells

The immune effector cells may be natural killer (NK) cells. Natural killer (NK) cells are a subpopulation of lymphocytes that have spontaneous cytotoxicity against a variety of tumor cells, virus-infected cells, and some normal cells in the bone marrow and thymus. NK cells are critical effectors of the early innate immune response toward transformed and virus-infected cells. NK cells constitute about 10% of the lymphocytes in human peripheral blood. When lymphocytes are cultured in the presence of interleukin 2 (IL-2), strong cytotoxic reactivity develops. NK cells are effector cells known as large granular lymphocytes because of their larger size and the presence of characteristic azurophilic granules in their cytoplasm. NK cells differentiate and mature in the bone marrow, lymph nodes, spleen, tonsils, and thymus. NK cells can be detected by specific surface markers, such as CD16, CD56, and CD8 in humans. NK cells do not express T-cell antigen receptors, the pan T marker CD3, or surface immunoglobulin B cell receptors.

Stimulation of NK cells is achieved through a cross-talk of signals derived from cell surface activating and inhibitory receptors. The activation status of NK cells is regulated by a balance of intracellular signals received from an array of germ-line-encoded activating and inhibitory receptors. When NK cells encounter an abnormal cell (e.g., tumor or virus-infected cell) and activating signals predominate, the NK cells can rapidly induce apoptosis of the target cell through directed secretion of cytolytic granules containing perforin and granzymes or engagement of death domain-containing receptors. Activated NK cells can also secrete type I cytokines, such as interferon-γ, tumor necrosis factor-α and granulocyte-macrophage colony-stimulating factor (GM-CSF), which activate both innate and adaptive immune cells as well as other cytokines and. Production of these soluble factors by NK cells in early innate immune responses significantly influences the recruitment and function of other hematopoietic cells. Also, through physical contacts and production of cytokines, NK cells are central players in a regulatory crosstalk network with dendritic cells and neutrophils to promote or restrain immune responses.

NK cells may be derived from human peripheral blood mononuclear cells (PBMC), unstimulated leukapheresis products (PBSC), human embryonic stem cells (hESCs), induced pluripotent stem cells (iPSCs), bone marrow, or umbilical cord blood by methods well known in the art. In certain aspects, the NK cells are isolated and expanded ex vivo. For example, CB mononuclear cells may be isolated by ficoll density gradient centrifugation and cultured in a bioreactor with IL-2 and artificial antigen presenting cells (aAPCs). After 7 days, the cell culture may be depleted of any cells expressing CD3 and re-cultured for an additional 7 days. The cells may be again CD3-depleted and characterized to determine the percentage of CD56⁺/CD3⁻ cells or NK cells. In other methods, umbilical CB may be used to derive NK cells by the isolation of CD34⁺ cells and differentiation into CD56⁺/CD3⁻ cells by culturing in medium contain SCF, IL-7, IL-15, and IL-2.

### F. Engineering of Immune Effector Cells

The immune effectors cells (e.g., autologous or allogeneic T cells (e.g., regulatory T cells, CD4+ T cells, CD8+ T cells, or gamma-delta T cells), NK cells, invariant NK cells, or NKT cells) may be genetically engineered to express antigen receptors such as chimeric antigen receptors (CARs). For example, the host cells (e.g., autologous or allogeneic T-cells) may be modified to express a CAR having antigenic specificity for coronavirus spike protein. In particular embodiments, NK cells are engineered to express a CAR. Multiple CARs, such as to different antigens, may be added to a single cell type, such as T cells or NK cells.

The cells may comprise one or more nucleic acids introduced via genetic engineering that encode one or more antigen receptors, and genetically engineered products of such nucleic acids. The nucleic acids may be heterologous, i.e., normally not present in a cell or sample obtained from the cell, such as one obtained from another organism or cell, which for example, is not ordinarily found in the cell being engineered and/or an organism from which such cell is derived. The nucleic acids may not be naturally occurring, such as a nucleic acid not found in nature (e.g., chimeric).

### V. Pharmaceutical Formulations

The present disclosure provides pharmaceutical compositions comprising antibodies that selectively target coronavirus spike protein. Such compositions comprise a prophylactically or therapeutically effective amount of an antibody or a fragment thereof and a pharmaceutically acceptable carrier. Also provided herein are pharmaceutical compositions and formulations comprising immune cells (e.g., T cells or NK cells) expressing a CAR and a pharmaceutically acceptable carrier.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. Water is a particular carrier when the pharmaceutical composition is administered intravenously. Saline solutions and aqueous dextrose and glycerol solutions can also be employed as liquid carriers, particularly for injectable solutions. The pH and exact concentration of the various components in a pharmaceutical composition are adjusted according to well-known parameters.

The active ingredients can be formulated for parenteral administration, e.g., formulated for injection via the intravenous, intramuscular, sub-cutaneous, or even intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The therapeutic compositions of the present embodiments are advantageously administered in the form of injectable compositions either as liquid solutions or suspensions; solid forms suitable for solution in, or suspension in, liquid prior to injection may also be prepared. These preparations also may be emulsified.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaceous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulations can include standard carriers such as pharmaceutical grades of mannitol, lactose, starch, magnesium stearate, sodium saccharine, cellulose, magnesium carbonate, etc. Examples of suitable pharmaceutical agents are described in Remington's Pharmaceutical Sciences. Such compositions will contain a prophylactically or therapeutically effective amount of the antibody or fragment thereof, preferably in purified form, together with a suitable amount of carrier so as to provide the form for proper administration to the patient.

Passive transfer of antibodies generally will involve the use of intravenous or intramuscular injections. The forms of antibody can be as monoclonal antibodies. Such immunity generally lasts for only a short period of time, and there is also a potential risk for hypersensitivity reactions, and serum sickness, especially from gamma globulin of non-human origin. The antibodies will be formulated in a carrier suitable for injection, i.e., sterile and syringeable.

Generally, the ingredients of compositions of the disclosure are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

In certain embodiments, pharmaceutical compositions may comprise, for example, at least about 0.1% of an active ingredient. In other embodiments, an active ingredient may comprise between about 2% to about 75% of the weight of the unit, or between about 25% to about 60%, for example, and any range derivable therein.

The term “unit dose” or “dosage” refers to physically discrete units suitable for use in a subject, each unit containing a predetermined quantity of the therapeutic composition calculated to produce the desired responses discussed above in association with its administration, i.e., the appropriate route and treatment regimen. The quantity to be administered, both according to number of treatments and unit dose, depends on the effect desired. The actual dosage amount of a composition of the present embodiments administered to a patient or subject can be determined by physical and physiological factors, such as body weight, the age, health, and sex of the subject, the type of disease being treated, the extent of disease penetration, previous or concurrent therapeutic interventions, idiopathy of the patient, the route of administration, and the potency, stability, and toxicity of the particular therapeutic substance. For example, a dose may also comprise from about 1 g/kg/body weight to about 1000 mg/kg/body weight (this such range includes intervening doses) or more per administration, and any range derivable therein. In non-limiting examples of a derivable range from the numbers listed herein, a range of about 5 μg/kg/body weight to about 100 mg/kg/body weight, about 5 μg/kg/body weight to about 500 mg/kg/body weight, etc., can be administered. The practitioner responsible for administration will, in any event, determine the concentration of active ingredient(s) in a composition and appropriate dose(s) for the individual subject.

### VI. Methods of Treatment

Certain aspects of the present embodiments can be used to prevent or treat a disease or disorder associated with a coronavirus infection, such as a coronavirus infection or COVID-19. In certain embodiments, the compositions and methods of the present embodiments involve administering an antibody or an antibody fragment against coronavirus spike protein, optionally in combination with a second or additional therapy.

“Treatment” and “treating” refer to administration or application of a therapeutic agent to a subject or performance of a procedure or modality on a subject for the purpose of obtaining a therapeutic benefit of a disease or health-related condition. For example, a treatment may include administration of a pharmaceutically effective amount of at least one antibody that targets coronavirus spike protein, either alone or in combination with other therapies.

The term “subject” as used herein refers to any individual or patient to which the subject methods are performed. Generally, the subject is human, although as will be appreciated by those in the art, the subject may be an animal. Thus, other animals, including mammals, such as rodents (including mice, rats, hamsters, and guinea pigs), cats, dogs, rabbits, farm animals (including cows, horses, goats, sheep, pigs, etc.), and primates (including monkeys, chimpanzees, orangutans, and gorillas) are included within the definition of subject.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of a coronavirus infection may involve, for example, a reduction in viral load. Treatment of coronavirus may also refer to increasing the likely hood of survival of a subject with a severe coronavirus infection.

In addition to being used as a monotherapy, the antibodies of the present invention may also find use in combination therapies. Effective combination therapy may be achieved with a single composition or pharmacological formulation that includes both agents, or with two distinct compositions or formulations, administered at the same time, wherein one composition includes at least one antibody of this invention, and the other includes the second agent(s). Alternatively, the antibody therapy may precede or follow the other agent treatment by intervals ranging from minutes to months.

Various combinations may be employed, such as when an antibody of the present invention is “A” and “B” represents a secondary agent, non-limiting examples of which are described below:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

It is contemplated that other therapeutic agents may be used in conjunction with the treatments of the current invention. In some embodiments, the present invention contemplates the use of one or more other therapies for the treatment of COVID-19 include the use of a coronavirus protease inhibitor, anti-platelet drugs, an anti-coagulation agent, a human type I interferon, a corticosteroid, or remdesivir.

In some embodiments, the anti-platelet drug is aspirin, an ADP receptor antagonist (e.g., ticlopidine, clopidogrel, cangrelor, prasugrel, ticagrelor, thienopyridine), or a glycoprotein IIb/IIIa receptor inhibitor (e.g., abciximab, eptifibatide, ticofiban). In some embodiment, the anti-coagulation agent is rivaroxaban, apixaban, dipyridamole, cilostazol, atromentin, edoxaban, fondaprinux, betrixaban, letaxaban, eribaxaban, hirudin, a thrombin inhibitor (e.g., lepirudin, desirudin, dabigatran, bivalirudin, ximelagatran), argatroban, batroxobin, hementin, low molecular weight heparin, unfractionated heparin, vitamin E, or a vitamin K antagonist (e.g., warfarin (Coumadin), acenocoumarol, phenprocoumon, phenindione).

Human type I interferons (IFNs) are a large subgroup of interferon proteins that help regulate the activity of the immune system. The mammalian types are designated IFN-α (alpha), IFN-β (beta), IFN-κ (kappa), IFN-δ (delta), IFN-ε (epsilon), IFN-τ (tau), IFN-ω (omega), and IFN-ζ (zeta, also known as limitin). Type I interferons have shown efficacy against the replication of various viruses, included Zika virus, chikungunya virus, flaviviruses, and hepatitis C virus. “Interferon compounds” include interferon-alpha, interferon-alpha analogues, interferon-alpha derivatives, interferon-alpha conjugates, interferon beta, interferon-beta analogues, interferon-beta derivatives, interferon-beta conjugates and mixtures thereof. The whole protein or its fragments can be fused with other peptides and proteins such as immunoglobulins and other cytokines. Interferon-alpha and interferon-beta conjugates may represent, for example, a composition comprising interferon-beta coupled to a non-naturally occurring polymer comprising a polyalkylene glycol moiety. Preferred interferon compounds include Roferon®, Intron®, Alferon®, Infergen®, Omniferon®, Alfacon-1, interferon-alpha, interferon-alpha analogues, pegylated interferon-alpha, polymerized interferon-alpha, dimerized interferon-alpha, interferon-alpha conjugated to carriers, interferon-alpha as oral inhalant, interferon-alpha as injectable compositions, interferon-alpha as a topical composition, Roferon® analogues, Intron® analogues, Alferon® analogues, and Infergen® analogues, Omniferon® analogues, Alfacon-1 analogues, interferon beta, Avonex™, Betaseron™, Betaferon™, Rebif™, interferon-beta analogues, pegylated interferon-beta, polymerized interferon-beta, dimerized interferon-beta, interferon-beta conjugated to carriers, interferon-beta as oral inhalant, interferon-beta as an injectable composition, interferon-beta as a topical composition, Avonex™ analogues, Betaseron™ Betaferon™ analogues, and Rebif™ analogues. Alternatively, agents that induce interferon-alpha or interferon-beta production or mimic the action of interferon-alpha or interferon-beta may also be employed. Interferon inducers include tilorone, poly(I)-poly(C), imiquimod, cridanimod, bropirimine.

It is contemplated that other agents may be used in combination with certain aspects of the present invention to improve the therapeutic efficacy of treatment. These additional agents include anti-virals, corticosteroids (e.g., dexamethasone), chloroquine, hydroxychloroquine, remdesivir, favipiravir, lopinavir, and ritonavir.

### VII. The Coronavirus Spike Protein

The spike protein of SARS-CoV-2 plays an essential role in virus entry into host cells and thus a primary target by neutralizing antibodies. The spike protein comprises an N-terminal S1 subunit and a C-terminal S2 subunit, which are responsible for receptor binding and membrane fusion. The S1 subunit is further divided into the N-terminal domain, the receptor-binding domain (RBD), the subdomain 1 (SD1) and subdomain 2 (SD2), and the S2 subunit is further divided into the fusion peptide (FP), the heptad repeat 1 (HR1) and heptad repeat 2 (HR2). The spike binds to a cellular receptor through its RBD, which triggers a conformational change of the spike. The activated spike is cleaved by a protease (such as TMPRSS2 for SARS-CoV and SARS-CoV-2) at S1/S2 site to release the S1 subunit and expose the FP on S2 subunit. The HR1 and HR2 refold to the post-fusion conformation to drive membrane fusion 35. Due to the functionality and a higher immunogenicity of the S1, most neutralizing antibodies characterized for coronavirus to date target the S1 subunit. A major challenge is that the S2 conformation is highly dynamic during membrane fusion, making it difficult to prepare the spike protein antigen and generate effective immune responses against spike (e.g., produce neutralizing antibodies). Spike protein stabilizing strategies have been demonstrated herein by mutation of the spike protein coding sequence. Mutant proteins were expressed as detailed in the Examples.

TABLE A
SARS-CoV-2 Variant Classification and Definitions.

| Variant | |
|---|---|
| Name | |
| (Pango | |
| lineage) | Spike Protein Substitutions (see SEQ ID NO: 76) |
| B.1.525 | Q52R, A67V, V70I, Y144V, E484K, D614G, Q677H, F888L |
| B.1.526 | L5F, T95I, D253G, S477N, E484K, D614G, A701V |
| B.1.617.1 | T95I, E154K, L452R, E484Q, D614G, P681R |
| B.1.617.2 | T19R, L452R, T478K, D614G, P681R, D950N |
| P.2 | E484K, D614G, V1176F |
| B.1.1.7 | H69del, V70del, Y144del, N501Y, A570D, D614G, P681H, |
| | T716I, S982A, D1118H |
| B.1.351 | D80A, D215G, L242del, A243del, L244del, K417N, E484K, |
| | N501Y, D614G, A701V |
| B.1.427 | S13I, W152C, W258L, L452R, D614G |
| B.1.429 | S13I, P26S, W152C, L452R, D614G |
| P.1 | L18F, T20N, P26S, D138Y, R190S, K417T, E484K, N501Y, |
| | D614G, H655Y, T1027I, V1176F |
| B.1.1.529 | A67V, H69del, V70del, T95I, G142D, V143del, Y144del, |
| | Y145del, N211del, L212I, ins214EPE, G339D, S371L, |
| | S373P, S375F, K417N, N440K, G446S, S477N, T478K, |
| | E484A, Q493R, G496S, Q498R, N501Y, Y505H, T547K, |
| | D614G, H655Y, N679K, P681H, N764K, D796Y, N856K, |
| | Q954H, N969K, L981F |

### VIII. Methods of Detection

In some aspects, the present disclosure concerns immunodetection methods for detecting the presence of a coronavirus spike protein. A wide variety of assay formats are contemplated for detecting protein products, including immunohistochemistry, enzyme linked immunosorbent assay (ELISA), radioimmunoassay (RIA), immunoradiometric assay, fluoroimmunoassay, chemiluminescent assay, bioluminescent assay, dot blotting, FACS analyses, and Western blot to mention a few. The steps of various useful immunodetection methods have been described in the scientific literature. In general, the immunobinding methods include obtaining a sample, and contacting the sample with an antibody specific for the protein to be detected, as the case may be, under conditions effective to allow the formation of immunocomplexes. In general, the detection of immunocomplex formation is well known in the art and may be achieved through the application of numerous approaches. These methods are generally based upon the detection of a label or marker, such as any of those radioactive, fluorescent, biological and enzymatic tags. Of course, one may find additional advantages through the use of a secondary binding ligand such as a second antibody and/or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

As used herein, the term “sample” refers to any sample suitable for the detection methods provided by the present invention. The sample may be any sample that includes material suitable for detection or isolation. Sources of samples include blood, pleural fluid, peritoneal fluid, urine, saliva, malignant ascites, broncho-alveolar lavage fluid, synovial fluid, and bronchial washes. In one aspect, the sample is a blood sample, including, for example, whole blood or any fraction or component thereof. A blood sample suitable for use with the present invention may be extracted from any source known that includes blood cells or components thereof, such as venous, arterial, peripheral, tissue, cord, and the like. For example, a sample may be obtained and processed using well-known and routine clinical methods (e.g., procedures for drawing and processing whole blood). In one aspect, an exemplary sample may be peripheral blood drawn from a subject with cancer. In some aspects, the biological sample comprises a plurality of cells. In certain aspects, the biological sample comprises fresh or frozen tissue. In specific aspects, the biological sample comprises formalin fixed, paraffin embedded tissue. In some aspects, the biological sample is a tissue biopsy, fine needle aspirate, blood, serum, plasma, cerebral spinal fluid, urine, stool, saliva, circulating tumor cells, exosomes, or aspirates and bodily secretions, such as sweat. In some aspects, the biological sample contains cell-free DNA.

In still further embodiments, the present disclosure concerns immunodetection methods for binding, purifying, removing, quantifying and otherwise generally detecting Coronavirus S protein. While such methods can be applied in a traditional sense, another use will be in quality control and monitoring of vaccine stocks, where antibodies according to the present disclosure can be used to assess the amount or integrity (i.e., long term stability) of antigens. Alternatively, the methods may be used to screen various antibodies for appropriate/desired reactivity profiles.

Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. In particular, a competitive assay for the detection and quantitation of Coronavirus S protein also is provided. The steps of various useful immunodetection methods have been described in the scientific literature, such as, e.g., Doolittle and Ben-Zeev (1999), Gulbis and Galand (1993), De Jager et al. (1993), and Nakamura et al. (1987). In general, the immunobinding methods include obtaining a sample suspected of containing Coronavirus S protein, and contacting the sample with a first antibody in accordance with the present disclosure, as the case may be, under conditions effective to allow the formation of immunocomplexes.

These methods include methods for detecting or purifying Coronavirus S protein or Coronavirus S protein from a sample. The antibody will preferably be linked to a solid support, such as in the form of a column matrix, and the sample suspected of containing the Coronavirus S protein will be applied to the immobilized antibody. The unwanted components will be washed from the column, leaving the Coronavirus S protein-expressing cells immunocomplexed to the immobilized antibody, which is then collected by removing the organism or antigen from the column.

The immunobinding methods also include methods for detecting and quantifying the amount of Coronavirus S protein or related components in a sample and the detection and quantification of any immune complexes formed during the binding process. Here, one would obtain a sample suspected of containing Coronavirus S protein and contact the sample with an antibody that binds Coronavirus S protein or components thereof, followed by detecting and quantifying the amount of immune complexes formed under the specific conditions. In terms of antigen detection, the biological sample analyzed may be any sample that is suspected of containing Coronavirus S protein, such as a tissue section or specimen, a homogenized tissue extract, a biological fluid (e.g., a nasal swab), including blood and serum, or a secretion, such as feces or urine.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to Coronavirus S protein. After this time, the sample-antibody composition, such as a tissue section, ELISA plate, dot blot or Western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. Patents concerning the use of such labels include U.S. Pat. Nos. 3,817,837, 3,850,752, 3,939,350, 3,996,345, 4,277,437, 4,275,149 and 4,366,241. Of course, one may find additional advantages through the use of a secondary binding ligand such as a second antibody and/or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two-step approach. A second binding ligand, such as an antibody that has binding affinity for the antibody, is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection uses two different antibodies. A first biotinylated antibody is used to detect the target antigen, and a second antibody is then used to detect the biotin attached to the complexed biotin. In that method, the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

### A. ELISAs

Immunoassays, in their most simple and direct sense, are binding assays. Certain preferred immunoassays are the various types of enzyme linked immunosorbent assays (ELISAs) and radioimmunoassays (RIA) known in the art. Immunohistochemical detection using tissue sections is also particularly useful. However, it will be readily appreciated that detection is not limited to such techniques, and western blotting, dot blotting, FACS analyses, and the like may also be used.

In one exemplary ELISA, the antibodies of the disclosure are immobilized onto a selected surface exhibiting protein affinity, such as a well in a polystyrene microtiter plate. Then, a test composition suspected of containing the Coronavirus S protein is added to the wells. After binding and washing to remove non-specifically bound immune complexes, the bound antigen may be detected. Detection may be achieved by the addition of another anti-Coronavirus S protein antibody that is linked to a detectable label. This type of ELISA is a simple “sandwich ELISA.” Detection may also be achieved by the addition of a second anti-Coronavirus S protein antibody, followed by the addition of a third antibody that has binding affinity for the second antibody, with the third antibody being linked to a detectable label.

In another exemplary ELISA, the samples suspected of containing the Coronavirus S protein (e.g., potentially infected cells) are immobilized onto the well surface and then contacted with the anti-Coronavirus S protein antibodies of the disclosure. After binding and washing to remove non-specifically bound immune complexes, the bound anti-Coronavirus S protein antibodies are detected. Where the initial anti-Coronavirus S protein antibodies are linked to a detectable label, the immune complexes may be detected directly. Again, the immune complexes may be detected using a second antibody that has binding affinity for the first anti-Coronavirus S protein antibody, with the second antibody being linked to a detectable label.

Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. Preferably, this will be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated, e.g., using a visible spectra spectrophotometer.

### B. Western Blot

The Western blot (alternatively, protein immunoblot) is an analytical technique used to detect specific proteins in a given sample of tissue homogenate or extract. It uses gel electrophoresis to separate native or denatured proteins by the length of the polypeptide (denaturing conditions) or by the 3-D structure of the protein (native/non-denaturing conditions). The proteins are then transferred to a membrane (typically nitrocellulose or PVDF), where they are probed (detected) using antibodies specific to the target protein.

Samples may be taken from whole tissue or from cell culture. In most cases, solid tissues are first broken down mechanically using a blender (for larger sample volumes), using a homogenizer (smaller volumes), or by sonication. Cells may also be broken open by one of the above mechanical methods. Assorted detergents, salts, and buffers may be employed to encourage lysis of cells and to solubilize proteins. Protease and phosphatase inhibitors are often added to prevent the digestion of the sample by its own enzymes. Tissue preparation is often done at cold temperatures to avoid protein denaturing.

The proteins of the sample are separated using gel electrophoresis. Separation of proteins may be by isoelectric point (pI), molecular weight, electric charge, or a combination of these factors. The nature of the separation depends on the treatment of the sample and the nature of the gel. This is a very useful way to determine a protein. It is also possible to use a two-dimensional (2-D) gel which spreads the proteins from a single sample out in two dimensions. Proteins are separated according to isoelectric point (pH at which they have neutral net charge) in the first dimension, and according to their molecular weight in the second dimension.

In order to make the proteins accessible to antibody detection, they are moved from within the gel onto a membrane made of nitrocellulose or polyvinylidene difluoride (PVDF). The membrane is placed on top of the gel, and a stack of filter papers placed on top of that. The entire stack is placed in a buffer solution which moves up the paper by capillary action, bringing the proteins with it. Another method for transferring the proteins is called electroblotting and uses an electric current to pull proteins from the gel into the PVDF or nitrocellulose membrane. The proteins move from within the gel onto the membrane while maintaining the organization they had within the gel. As a result of this blotting process, the proteins are exposed on a thin surface layer for detection (see below). Both varieties of membrane are chosen for their non-specific protein binding properties (i.e., binds all proteins equally well). Protein binding is based upon hydrophobic interactions, as well as charged interactions between the membrane and protein. Nitrocellulose membranes are cheaper than PVDF, but are far more fragile and do not stand up well to repeated probings. The uniformity and overall effectiveness of transfer of protein from the gel to the membrane can be checked by staining the membrane with Coomassie Brilliant Blue or Ponceau S dyes. Once transferred, proteins are detected using labeled primary antibodies, or unlabeled primary antibodies followed by indirect detection using labeled protein A or secondary labeled antibodies binding to the Fc region of the primary antibodies.

### C. Immunohistochemistry

The antibodies of the present disclosure may also be used in conjunction with both fresh-frozen and/or formalin-fixed, paraffin-embedded tissue blocks prepared for study by immunohistochemistry (IHC). The method of preparing tissue blocks from these particulate specimens has been successfully used in previous IHC studies of various prognostic factors, and is well known to those of skill in the art (Brown et al., 1990; Abbondanzo et al., 1990; Allred et al., 1990).

Briefly, frozen-sections may be prepared by rehydrating 50 ng of frozen “pulverized” tissue at room temperature in phosphate buffered saline (PBS) in small plastic capsules; pelleting the particles by centrifugation; resuspending them in a viscous embedding medium (OCT); inverting the capsule and/or pelleting again by centrifugation; snap-freezing in −70° C. isopentane; cutting the plastic capsule and/or removing the frozen cylinder of tissue; securing the tissue cylinder on a cryostat microtome chuck; and/or cutting 25-50 serial sections from the capsule. Alternatively, whole frozen tissue samples may be used for serial section cuttings.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections. Again, whole tissue samples may be substituted.

### D. Immunodetection Kits

In still further embodiments, the present disclosure concerns immunodetection kits for use with the immunodetection methods described above. As the antibodies may be used to detect Coronavirus S protein, the antibodies may be included in the kit. The immunodetection kits will thus comprise, in suitable container means, a first antibody that binds to an Coronavirus S protein, and optionally an immunodetection reagent.

In certain embodiments, the antibody may be pre-bound to a solid support, such as a column matrix and/or well of a microtiter plate. The immunodetection reagents of the kit may take any one of a variety of forms, including those detectable labels that are associated with or linked to the given antibody. Detectable labels that are associated with or attached to a secondary binding ligand are also contemplated. Exemplary secondary ligands are those secondary antibodies that have binding affinity for the first antibody.

Further suitable immunodetection reagents for use in the present kits include the two-component reagent that comprises a secondary antibody that has binding affinity for the first antibody, along with a third antibody that has binding affinity for the second antibody, the third antibody being linked to a detectable label. As noted above, a number of exemplary labels are known in the art and all such labels may be employed in connection with the present disclosure.

The kits may further comprise a suitably aliquoted composition of Coronavirus S protein, whether labeled or unlabeled, as may be used to prepare a standard curve for a detection assay. The kits may contain antibody-label conjugates either in fully conjugated form, in the form of intermediates, or as separate moieties to be conjugated by the user of the kit. The components of the kits may be packaged either in aqueous media or in lyophilized form.

The container means of the kits will generally include at least one vial, test tube, flask, bottle, syringe or other container means, into which the antibody may be placed, or preferably, suitably aliquoted. The kits of the present disclosure will also typically include a means for containing the antibody, antigen, and any other reagent containers in close confinement for commercial sale. Such containers may include injection or blow-molded plastic containers into which the desired vials are retained.

### E. Flow Cytometry and FACS

The antibodies of the present disclosure may also be used in flow cytometry or FACS. Flow cytometry is a laser- or impedance-based technology employed in many detection assays, including cell counting, cell sorting, biomarker detection and protein engineering. The technology suspends cells in a stream of fluid and passing them through an electronic detection apparatus, which allows simultaneous multiparametric analysis of the physical and chemical characteristics of up to thousands of particles per second. Flow cytometry is routinely used in the diagnosis disorders, especially blood cancers, but has many other applications in basic research, clinical practice and clinical trials.

Fluorescence-activated cell sorting (FACS) is a specialized type of cytometry. It provides a method for sorting a heterogenous mixture of biological cells into two or more containers, one cell at a time, based on the specific light scattering and fluorescent characteristics of each cell. In general, the technology involves a cell suspension entrained in the center of a narrow, rapidly flowing stream of liquid. The flow is arranged so that there is a large separation between cells relative to their diameter. A vibrating mechanism causes the stream of cells to break into individual droplets. Just before the stream breaks into droplets, the flow passes through a fluorescence measuring station where the fluorescence of each cell is measured. An electrical charging ring is placed just at the point where the stream breaks into droplets. A charge is placed on the ring based immediately prior to fluorescence intensity being measured, and the opposite charge is trapped on the droplet as it breaks form the stream. The charged droplets then fall through an electrostatic deflection system that diverts droplets into containers based upon their charge.

In certain embodiments, to be used in flow cytometry or FACS, the antibodies of the present disclosure are labeled with fluorophores and then allowed to bind to the cells of interest, which are analyzed in a flow cytometer or sorted by a FACS machine.

### IX. Kits

In various aspects of the embodiments, a kit is envisioned containing therapeutic agents and/or other therapeutic and delivery agents. In some embodiments, a kit is provided for preparing and/or administering a therapy of the embodiments. The kit may comprise one or more sealed vials containing any of the pharmaceutical compositions of the present embodiments. The kit may include, for example, at least one coronavirus spike protein-specific antibody or coronavirus spike protein-specific CAR construct, as well as reagents to prepare, formulate, and/or administer the components of the embodiments or perform one or more steps of the inventive methods. In some embodiments, the kit may also comprise a suitable container, which is a container that will not react with components of the kit, such as an eppendorf tube, an assay plate, a syringe, a bottle, or a tube. The container may be made from sterilizable materials such as plastic or glass.

In still further embodiments, the present disclosure concerns immunodetection kits for use with the immunodetection methods described above. As the antibodies may be used to detect a coronavirus or a coronavirus antigen, the antibodies may be included in the kit. The immunodetection kits will thus comprise, in suitable container means, a first antibody that binds to a coronavirus or a coronavirus antigen, and optionally an immunodetection reagent.

In certain embodiments, the coronavirus spike protein-specific antibody may be pre-bound to a solid support, such as a column matrix and/or well of a microtiter plate. The immunodetection reagents of the kit may take any one of a variety of forms, including those detectable labels that are associated with or linked to the given antibody. Detectable labels that are associated with or attached to a secondary binding ligand are also contemplated. Exemplary secondary ligands are those secondary antibodies that have binding affinity for the first antibody.

Further suitable immunodetection reagents for use in the present kits include the two-component reagent that comprises a secondary antibody that has binding affinity for the first antibody, along with a third antibody that has binding affinity for the second antibody, the third antibody being linked to a detectable label. As noted above, a number of exemplary labels are known in the art and all such labels may be employed in connection with the present disclosure.

The kits may further comprise a suitably aliquoted composition of the coronavirus or the coronavirus antigens, whether labeled or unlabeled, as may be used to prepare a standard curve for a detection assay. The kits may contain antibody-label conjugates either in fully conjugated form, in the form of intermediates, or as separate moieties to be conjugated by the user of the kit. The components of the kits may be packaged either in aqueous media or in lyophilized form.

The kit may further include an instruction sheet that outlines the procedural steps of the methods set forth herein, and will follow substantially the same procedures as described herein or are known to those of ordinary skill in the art. The instruction information may be in a computer readable media containing machine-readable instructions that, when executed using a computer, cause the display of a real or virtual procedure of delivering a pharmaceutically effective amount of a therapeutic agent.

### X. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Materials & Methods

Spike expression. Soluble coronavirus spikes and spike variants were expressed and purified as previously described (Wrapp et al., 2020; Hsieh et al., 2020). SARS-CoV-2 (Wrapp et al., 2020), SARS-CoV (Kirchdoerfer et al., 2018), and SARS-CoV-2 HexaPro (Hsieh et al., 2020) spikes and epitope variants, HexaPro E1031R, and monoHP were expressed in ExpiCHO cells (ThermoFisher Scientific). The monoHP variant was produced by adding a short linker and a HRV3C cleavage site between the C-terminus of SARS-CoV-2 HexaPro and the T4 fibritin domain. The purified protein was incubated at 4° C. for ˜5 days and incubated overnight with HRV3C protease. The monoHP spike was purified by SEC from remaining trimer, HRV3C protease and tags. MERS-CoV (Pallesen et al., 2017), HKU1 (Pallesen et al., 2017), and the SARS-CoV-2 variants HexaPro S2 (residues 697-1208 of the SARS-CoV-2 spike with an artificial signal peptide, proline substitutions at positions 817, 892, 899, 942, 986 and 987 and a C-terminal T4 fibritin domain, HRV3C cleavage site, 8×HisTag and TwinStrepTag), HexaPro RBD-locked-down (HexaPro with S383C-D985C substitutions), and aglycosylated HexaPro (HexaPro treated with Endo H overnight at 4° C. leaving only one N-acetylglucosamine attached to N-glycosylation site) as well as MERS-CoV S2-only (residues 763-1291 of MERS-2P with 8 additional stabilizing substitutions), MERS-CoV S2-apex-less (MERS-CoV S2-only construct with residues 811-824 replaced with GGSGGS and residues 1042-1073 replaced with a flexible linker; the 3A3 epitope spans residues 1052-1080 in MERS) were expressed in Freestyle 293-F cells (ThermoFisher Scientific).

Murine immunization. Three BALB/c mice were immunized sub-cutaneously with 5 μg pre-fusion stabilized MERS-CoV S2 and 20 μg of ODN1826+100 μl of 2× Sigma Adjuvant System (SAS; Sigma) containing monophosphoryl lipid A and trehalose dimycolate in squalene oil. Four weeks later, the mice were boosted with the same dose of the same mixture. Three weeks after boosting, the mice were sacrificed and spleens were collected in RNALater (ThermoFisher). Mouse protocols were approved by the University of Texas at Austin IACUC (AUP-2018-00092).

Phage display antibody library construction. RNA was isolated from the aqueous phase of homogenized spleens mixed with 1-bromo-3-chloropropane and purified with the PureLink RNA kit (Invitrogen) separately. The Superscript IV kit (Invitrogen) was used to synthesize cDNA, the V_{H} and V_{L} sequences from each immunized mouse were amplified with mouse-specific primers described by Krebber et al (1997). Maintaining separate reactions for each mouse, the V_{L} and V_{H} regions were joined by overlap extension PCR for each immunized mouse spleen to generate VL-linker-VH fragments (scFv), in which the linker region encodes the amino acids (Gly₄Ser)₄ and SfiI sites flanked the scFv sequence. The scFv PCR products were pooled cloned into pMopac24 (Hayhurst et al., 2003) via SfiI cut sites to encode the M13 phage pIII protein fused to an scFv with a c-terminal myc tag. This library was then transformed to XL1-Blue (Agilent Technologies) E. coli. The total number of transformants was 3.1×10⁸ with <0.01% background based on plating.

Phage display and panning. The E. coli containing the library were expanded in growth media (2×YT with 1% glucose, 200 μg/mL ampicillin, 10 μg/mL tetracycline) at 37° C. to an OD₆₀₀ of 0.5, then infected with 1×10¹¹ pfu/ml M13K07 helper phage (NEB) and induced with 1 mM isopropyl β-d-1-thiogalactopyranoside. After two hours of shaking at room temperature, 12.5 μg/mL of kanamycin was added for phage expression overnight. Phage were precipitated in 20% PEG-8000 in 2.5 M NaCl, titered by infection of XL1-Blue and plating, and used for Round 1 panning. This process was repeated for each round of panning starting from overnight growth of the output phage from each round.

Four rounds of panning were used to isolate scFvs binding both MERS-CoV S2 and SARS-CoV-2 spike using the following solutions coated on high binding plates: 2 μg/mL anti-c-myc tag antibody (Invitrogen) to eliminate phage expressing no or truncated scFv (Round 1), 2 μg/mL MERS-CoV S2 (Round 2), 2 μg/mL SARS-CoV-2 spike (Round 3), and 0.4 μg/mL SARS-CoV-2 spike (Round 4). In each round of panning, the plates were blocked with 5% non-fat milk in phosphate buffered saline (PBS) with 0.05% Tween-20 (PBS-T), and phage were preincubated with 5% non-fat milk in PBS-T for 30 minutes prior to incubation on the plate for 1.5 h at room temperature. After thorough washing with PBS-T, output phage were eluted using 0.1 M HCl at pH 2.2, neutralized with ˜1:20 2M Tris base, and allowed to infect XL1-Blue cells for overnight amplification.

Random clones isolated after Round 3 and Round 4 of panning were sequenced and unique clones were tested by monoclonal phage enzyme-linked immunosorbent assay (ELISA) on plates coated with SARS-CoV-2 spike or RSV F foldon at 2 μg/mL in PBS. Briefly, plates were coated overnight at 4° C., washed with PBS-T, then blocked with PBS-T and 5% milk. Phage were allowed to bind for one hour at room temperature, thoroughly washed with PBS-T, then incubated with 1:2000 anti-M13 pVIII-HRP (GE Healthcare) in PBS-T and 5% milk for another hour. After washing, the plate was developed with the TMB Substrate Kit (Thermo Scientific), quenched with an equal volume of 1 M HCl and evaluated by absorbance at 450 nm (FIG. 9).

Antibody expression, purification, and quality control. Full-length antibody versions of 3A3, 4A5, 4H2, and 3E11 were cloned as previously described (Nguyen et al., 2015) as mouse variable region-human IgG1 constant region chimeras. Antibodies were expressed in ExpiCHO (ThermoFisher Scientific) cells according to the high titer protocol provided, and purified on a Protein A HiTrap column (GE Healthcare) with the ACTA Pure FPLC system (GE Healthcare), and buffer exchanged to PBS. Each purified antibody was analyzed by SDS-PAGE (3 μg antibody per well) under reducing and non-reducing conditions (FIG. 10A), and by analytical size exclusion chromatography on a Superdex S200 column (GE Healthcare) (FIG. 10B).

Mouse Fab fragments of each sequence were generated by cloning the V_{H} regions into a plasmid containing the constant regions with a 3C protease site in the hinge and the V_{L} regions into a mouse kappa chain expression cassette in the pAbVec background (Wagner et al. 2019). After expression, protein A purified protein was digested with human rhinovirus 3C protease, and the flow-through from a protein A HiTrap column was collected. Excess 3C protease was removed by incubation with Ni Sepharose 6Fast Flow beads (GE Healthcare). Fully murine antibodies were produced by cloning the V_{H} regions into a mouse IgG2 expression cassette in the pAbVec background, co-transfected with the appropriate mouse IgK plasmid (Wagner et al., 2019), and purified as described above.

According to the kit instructions, the thermal unfolding temperatures of the chimeric antibodies (0.3 mg/ml) were assessed in triplicate using the Protein Thermal Shift Dye Kit (ThermoFisher Scientific). Continuous fluorescence measurements (λₑₓ=580 nm, λₑₘ=623 nm) were performed using a ThermoFisher ViiA 7 Real-Time PCR System, with a temperature ramp rate of 0.05° C./sec increasing from 25° C. to 99° C. (Tables 9 and 10, FIG. 10C).

ELISA evaluation of antibody cross-reactivity and binding to stressed spike. ELISAs were performed as described above throughout the work. For testing each antibody's specificity, plates were coated with 1 μg/mL of purified spike proteins (SARS-CoV, SARS-CoV-2, SARS-CoV-2 HexaPro, MERS-CoV, HKU1, and RSV F foldon) in PBS. Duplicate serial dilutions of each full-length antibody were allowed to bind each coat, and the secondary antibody solution was a 1:1200 dilution of goat-anti-human IgG Fc-HRP (SouthernBiotech). ELISA curves were fit to a 4 parameter logistic curve (FIGS. 1A and 12).

To stress the spike proteins, fresh aliquots of SARS-CoV-2 and SARS-CoV-2 HexaPro spikes were thawed and split. One half of the aliquot was stressed by incubation at −20° C. for 5 min, then 50° C. for 2 min for a total of three temperature cycles. The freshly thawed and stressed spikes were serially diluted and captured on ELISA plates coated with each full-length antibody at 1 μg/mL or nothing (no coat). The ELISA was carried out as above with 3% w/v BSA in place of milk in the diluent and blocking buffer and Strep-Tactin-HRP (IBA) as the secondary reagent (FIGS. 1C, 1D). For each fresh and stressed spike, 8 μg was analyzed by SDS-PAGE under non-reducing conditions (FIG. 13).

Western blot of antibody binding to coronavirus spike proteins. Purified coronavirus spike proteins (SARS-CoV-2 HexaPro, SARS-CoV-2, MERS-CoV, and HKU1) were reduced and boiled, and 50 ng of each was subjected to SDS-PAGE and transfer to PVDF membranes in quadruplicate. After blocking with PBS-T with 5% milk, the membranes were probed with 0.2 μg/mL 3A3, 1 μg/mL 4A5, 1 μg/mL 4H2 or 0.2 μg/mL 3E11 for 1 h at room temperature. After washing with PBST, the membranes were incubated with 1:4000 goat anti-human IgG Fc-HRP for 45 min at room temperature, then developed with the SuperSignal West Pico Chemiluminescent Substrate (Thermo Scientific) and imaged (FIG. 1B).

Surface plasmon resonance (SPR) and biolayer interferometry (BLI) measurements. SPR was used to determine the binding kinetics and affinity of the 3A3 Fab and HexaPro S2 interaction. An anti-StrepTagII Fab was covalently coupled to a CM5 sensor chip, which was then used to capture purified HexaPro S2 by the c-terminal twin StrepTag to ˜80 response units (RU) in each cycle using a Biacore X100 (GE Healthcare). The binding surface was regenerated between cycles using 0.1% SDS followed by 10 mM Glycine at pH 2. The 3A3 Fab was serially diluted from 12.5 nM to 1.56 nM and injected over the blank reference flow cell and then HexaPro S2-coated flow cell in HBS-P+ buffer. Buffer was also injected through both flow cells as a reference. The data were double-reference subtracted and fit to a 1:1 binding model using BIAevaluation software.

To determine the affinity of 3A3 Fab by BLI, anti-human IgG Fc (AHC) (ForteBio) sensors were coated with the anti-foldon antibody identified in this work (3E11) at 10 nM in the kinetics buffer (0.01% BSA and 0.002% Tween-20 in PBS) until a response of 0.6 nm. MAb-coated sensors were then incubated with HexaPro S2 at 60 nM until a response of 0.6 nm. Association of 3A3 Fab was recorded for 5 minutes in kinetics buffer, starting at 100 nM followed by 1:2 dilutions. The dissociation was recorded for 10 minutes in the kinetics buffer. K_{d} values were obtained using a 1:1 global fit model using the Octet instrument software. 3A3 Fab kinetics measurement was repeated once (FIG. 11B, Tables 9 and 10).

Apparent K_{d}s of IgGs were measured using the Octet Red96 (ForteBio) instrument. Anti-Human IgG Fc (AHC) (ForteBio) sensors were loaded with mAbs in kinetic buffer (0.01% BSA and 0.002% Tween-20 in PBS) at 10 nM until a response of 0.6 nm was reached. Association curves were recorded for 5-30 min by incubating the sensors in different concentrations of SARS-CoV-2 HexaPro, SARS-CoV-2, aglycosylated HexaPro, starting from 100 nM, and serial 1:2 dilutions. Dissociation step was recorded for 10-20 min in the kinetic buffer. Steady state K_{d} values were determined using the response values obtained at the five-minute mark of the association step using the Octet analysis software (Tables 9 and 10, FIGS. 11C and 11D). To evaluate spikes incubated at different temperatures, the spikes were stored for ˜1-3 weeks at 4° C. or 24 hours at 37° C., then diluted into room temperature buffer immediately before measurements.

To compare 3A3 and mAb 2-4 binding to HexaPro and “Down” HexaPro, anti-Human IgG Fc (AHC) (ForteBio) sensors were loaded with 3A3 mAb or mAb 2-4 in kinetic buffer at 10 nM until a response of 0.6 nm was reached. After a baseline step, the sensors were briefly incubated with either HexaPro or “Down” HexaPro, both at 60 nM. Short dissociation step recorded in kinetic buffer (FIG. 6A).

To determine the affinity of 3A3 Fab, Anti-human IgG Fc sensors were coated with anti-fold on antibody (3E11) at 20 nM in kinetic buffer. MAb coated sensors were then incubated with HexaPro S2 at 60 nM until a response of 0.6 nm was obtained. Association of 3A3 Fab was recorded for 5 minutes in kinetics buffer, starting at 100 nM followed by 1:2 dilutions. Dissociation was recorded for 10 minutes in kinetics buffer. K_{d} values were obtained using a 1:1 global fit model using the Octet instrument software. 3A3 Fab kinetics measurement was repeated once.

To evaluate ACE2 binding to HexaPro captured by 3A3, Anti-Human Fc Sensors were used to pick up 3A3 (10 nM) to a response of 0.6 nm. Then mAb coated tips were dipped into wells containing HexaPro (50 nM) to a response of 0.6 nm and then dipped into wells containing ACE2 (50 nM), irrelevant murine mAb (50 nM), or buffer. Association of mu3A3/irrelevant mAb was measured for 5 min and dissociation for 10 min. (FIG. 7B). Octet Red96 (ForteBio) instrument was used. Between every loading step, sensors were washed with kinetics buffer for 1 min. Before use, sensors were hydrated in kinetics buffer for 10 minutes. After each assay, the sensors were regenerated using 10 mM Glycine, pH 1.5.

Confocal cell fusion assay. On day 0, the CHO-T cells (Acyte Biotech) were transfected with either pPyEGFP (Nguyen et al., 2018), 1:4 pWT-SARS-CoV-2-spike:pPyEGFP, and 1:4 pD614G-SARS-CoV-2-spike:pPyEGFP using Lipofectamine 2000 (Life Technologies), and media was replaced on day 1. On day 2 after transfection, HEK-293T-hACE2 cells (BEI, NR-52511), which stably expresses human ACE2, were stained with 1 μM CellTrace Far Red dye (Invitrogen, Ex/Em: 630/661 nm) in PBS for 20 min at room temperature, then quenched with DMEM with 10% heat-inactivated FBS for 5 min, and resuspended in fresh media. CHO-T cells expressing EGFP or EGFP and surface spike were preincubated with antibody for one hour at 37° C., then mixed with HEK-hACE2 cells at a ratio of 5:1 in 24-well plates with a coverslip on the bottom of each well. On day 3, after 20 h of coincubation, the coverslip with bound cells was washed once with PBS and fixed with 4% paraformaldehyde for 20 min at room temperature, washed again, and mounted on slides with DAPI-fluoromount-G (Southern Biotech). Images were collected with Zeiss LSM 710 confocal microscope (Carl Zeiss, Inc) and processed using ImageJ software (available at rsbweb.nih.gov/ij) (FIG. 2).

The cell fusion level was determined by two different statistical analysis methods. The first statistical analysis was based on the percentage of HEK-ACE2 pixels (red) colocalizing with spike expressing CHO pixels (green), which was determined by the following equation within the JACoP plugin for ImageJ (Bolte & Cordelieres, 2006):

$$HEK - ACE 2 colocalization \% = \frac{( \begin{matrix}summed intensities at 633 nm wavelength of \\ HEK - ACE 2 pixels colocalizaing with CHO pixels\end{matrix} )}{( \begin{matrix}summed intensities at 633 nm wavelength \\ of HEK - ACE 2 pixels\end{matrix} )}$$

The colocalization percentage for each independent image was determined using the Manders' coefficient. The second statistical analysis was based on the average HEK-ACE2 cell size after the coincubation with CHO cells using the ImageJ software. The image of HEK-ACE2 (red fluorescence color at 633 nm wavelength) was converted into 16-bit in greyscale and adjusted the threshold to highlight the cell structure. The average cell size was automatically counted with the “Analyze Particles” tab with a size threshold (50-infinity) to exclude the background noise. The cell on the edge was excluded. The statistical significance of either HEK-ACE2 colocalization percentage or average cell size between different conditions was calculated with ANOVA using GraphPad Prism 7 (GraphPad Software). Values represent the mean and standard deviation of at least 160 cells.

Lentiviral plasmids. Plasmids required for lentiviral production were obtained from BEI Resources. Plasmids expressing the HIV virion under the CMV promotor (HDM-Hgpm2, pRC-CMV-Rev1b, and HDM-tat1b) were provided under the following catalog numbers NR-52516, NR-52519, and NR-52518, respectively (Crawford et al., 2020). Plasmids for lentiviral backbone expressing a luciferase reporter under the CMV promotor followed by an IRES and ZsGreen (pHAGE-CMV-Luc2-IRES-ZsGreen-W) or human ACE2 gene (GenBank ID NM_021804) under an EFla promoter (pHAGE2-EFlaInt-ACE2-WT) were provided as NR-52520 and NR52516, respectively (Crawford et al., 2020). The envelop vector expressing a codon-optimized WT SARS-CoV-2 spike protein (Genbank ID NC_045512) under a CMV promoter was obtained from BEI resources (HDM-IDTSpike-fixK, NR-52514) (Crawford et al., 2020), while the plasmid expressing VSV-G (vesicular stomatitis virus glycoprotein) was purchased from Cell Biolabs (pCMV-VSV-G, Part No. RV-110). The HDM-IDTSpike-fixK plasmid was employed as a template for site-directed mutagenesis to generate the expression plasmid for the D614G mutant of SARS-CoV-2 spike protein. SARS-CoV and B.1.1.7 spikes were cloned into the same HDM-IDTSpike-fixK plasmid for pseudovirus production.

Generation of HEK293T-ACE2 target cells, stably expressing human ACE2. A lentiviral vector (pHAGE2-EFlalnt-ACE2-WT) expressing human ACE2 under an EFla promoter was used to transduce HEK293T cells. Clonal selection depended on the susceptibility to infection by the pseudotyped lentiviral particles; selected clones were validated using western blotting.

SARS-CoV-2 spike-mediated pseudovirus entry assay. HIV particles pseudotyped with wild type or D614G mutant of SARS-CoV-2 spike were generated in HEK293T cells. A detailed protocol for generating these particles was reported by Crawford et al. (2020). HEK293T cells were co-transfected with plasmids for (1) HIV virion-formation proteins (HDM-Hgpm2, pRC-CMV-Rev1b, and HDM-tat1b; (2) lentiviral backbone expressing luciferase reporter (pHAGE-CMV-Luc2-IRES-ZsGreen-W), and (3) a plasmid encoding one of the envelope proteins (2019-nCoV Spike-WT, D614G mutant or VSV G as a positive control). 72 hours post-transfection, media containing the pseudovirus particles were collected, filtered, fractionated, and stored at −80° C. The particles were used directly in cell entry experiments or after pre-incubation with each antibody for one hour at room temperature. After 60-72 hours, a total number of cells per well were estimated using an lncuCyte® ZOOM equipment with a ×10 objective. Then cells were treated with the Bright-Glo Luciferase Assay reagent (Promega, E2610) to detect a luciferase signal (relative luciferase units or RLU) following the manufacturer's protocol. The percentage of entry was estimated as the ratio of the relative luciferase units recorded in the presence and absence of the tested antibody and a half-maximal inhibitory concentrations (IC₅₀) calculated using a 3-parameter logistic regression equation (GraphPad Prism v9.0) (FIG. 2).

Flow cytometry. On day 0, Expi-293 cells (ThermoFisher) were mock transfected or transfected with pWT-SARS-CoV-2-spike or pD614G-SARS-CoV-2-spike from the pACGGS expression vector (Sauer et al., 2020). On day 2, 30 nM full-length 3A3 was added to ˜5×10⁵ transfected cells for 1 h on ice. All cells were collected, washed with PBS with 1% FBS, then incubated with 1:100 goat-anti-human Fc-AF647 for one hour on ice. Cells were washed again, then scanned for AF647 (640 nm excitation, 670/30 bandpass emission) fluorescence on a BD Fortessa flow cytometer and analyzed with FlowJo (FIG. 6F).

Hydrogen-Deuterium Exchange Mass Spectrometry. Hydrogen-deuterium exchange was performed on 0.50 μM SARS-CoV-2 HexaPro spike protein alone (FIGS. 15 and 16) or in the presence of 0.55 μM 3A3 IgG or Fab (FIGS. 4 and 17), 0.75 μM 4A5, or 0.75 μM 4H2 (FIG. 18). Complexes were incubated for 10 min at 25° C. before exchange in 90% deuterium and 20 mM Tris pD 8.0, 200 mM NaCl. Exchange was quenched after 101, 10², 10³ and 10⁴ s, by mixing samples 1:1 with cooled 0.2% (v/v) Formic acid, 200 mM TCEP, 8 M Urea, pH 2.3. Samples were immediately flash-frozen in liquid N₂ and stored at −80° C.

Samples were thawed and LC/MS performed using a Waters HDX manager and SYNAPT G2-Si Q-Tof. Three or four technical replicates of each sample were analyzed in a random order. Samples were digested on-line by Sus scrofa Pepsin A (Waters Enzymate™ BEH Pepsin column) at 15° C. and peptides trapped on a C18 pre-column (Waters ACQUITY UPLC BEH C18 VanGuard pre-column) at 1° C. for 3 min at 100 μL/min. Peptides were separated over a C18 column (Waters ACQUITY UPLC BEH C18 column) and eluted with a linear 3-40% (v/v) Acetonitrile gradient for 7 min at 30 uL/min at 1° C. and 0.1% (v/v) Formic Acid as the basic LC buffer.

MS data were acquired using positive ion mode and either HDMS or HDMS^{E}. HDMS^{E} mode was used to collect both low (6 V) and high (ramping 22-44 V) energy fragmentation data for peptide identification in water-only samples. HDMS mode was used to collect low energy ion data for all deuterated samples. All samples were acquired in resolution mode. Capillary voltage was set to 2.8 kV for the sample sprayer. Desolvation gas was set to 650 L/hour at 175° C. The source temperature was set to 80° C. Cone and nebulizer gas was flowed at 90 L/hour and 6.5 bar, respectively. The sampling cone and source offset were both set to 30 V. Data were acquired at a scan time of 0.4 s with a range of 100-2000 m/z. Mass correction was done using [Glu1]-fibrinopeptide B as a reference mass.

Water-only control samples were processed by Protein Lynx Global Server v.3.0.2 with a “minimum fragment ion matches per peptide” of 3 and allowing methionine oxidation. The low and elevated energy thresholds were 250 and 50 counts, respectively, and overall intensity threshold was 750 counts. Resulting peptide lists were then used to search data from deuterated samples using DynaX v.3.0. Peptide filters of 0.3 products per amino acid and 1 consecutive product were used. Spectra were manually assessed, and figures prepared using HD-eXplosion (Zhang et al., 2020) and PyMOL (DeLano, 2002). The HDX data summary are provided in Table 8.

TABLE 8
HDX summary table

| | Data Set | | | | | |
|---|---|---|---|---|---|---|
| | | SARS- | SARS- | | SARS- | SARS- |
| | SARS- | CoV-2 | CoV-2 | SARS- | CoV-2 | CoV-2 |
| | CoV-2 | HexaPro | HexaPro | CoV-2 | HexaPro | HexaPro |
| | HexaPro | spike + | spike + | HexaPro | spike + | spike + |
| | spike | 3A3 IgG | 3A3 Fab | spike | 4A5 IgG | 4H2 IgG |
| | HDX reaction details | | | | | |
| | 200 mM NaCl, 20 mM Tris | | | 200 mM NaCl, 20 mM Tris | | |
| | | 0.5 uM S2 + | 0.5 uM S2 + | | 0.5 uM S2 + | 0.5 uM S2 + |
| | 0.5 uM | 0.55 uM | 0.55 uM | 0.5 uM | 0.75 uM | 0.75 uM |
| | S2 | 3A3 IgG | 3A3 Fab | S2 | 4A5 IgG | 4H2 IgG |
| | pHread = 7.6 | | | pHread = 7.6 | | |
| HDX time | 10, 100, 1000, 10000 at 25° C. | | | 10, 100, 1000, 10000 at 25° C. | | |
| course (s) | | | | | | |
| HDX control | Unlabeled S2 | | | Unlabeled S2 | | |
| samples | | | | | | |
| Back-exchange | ~40% | | | ~40% | | |
| (mean) | | | | | | |
| # of peptides | 192 | | | 188 | | |
| Sequence | 56.30% | | | 60.00% | | |
| coverage | | | | | | |
| Average | 13/3.34 | | | 13/3.21 | | |
| Peptide length/ | | | | | | |
| Redundancy | | | | | | |
| Replicates | 4 (technical) | | | 4 (technical) | | |
| (biological or | | | | | | |
| technical) | | | | | | |

| | | |
|---|---|---|
| Repeatability | 0.10 Da (average standard | 0.087 Da (average standard |
| | deviation) | deviation) |
| Significant | Average ΔHDX greater than 0.2 Da | Average ΔHDX greater than 0.2 Da |
| difference | and p-value less than 0.01 | and p-value less than 0.01 |

Statistical analyses. The means±SD were determined for all appropriate data. For the mammalian cell fusion experiments, pseudovirus neutralization experiments and epitope variant analysis, a one-way analysis of variance (ANOVA) with Tukey's simultaneous test with P values was used to determine statistical significance between groups. Welch's t-test was used to determine the significance of deuterium uptake differences.

### Example 1—Isolation of Antibodies Binding the S2 Domain of SARS-CoV, SARS-CoV-2, and MERS-CoV Spikes

These experiments primarily used two-proline stabilized spikes (S-2P). In this work, “spike” refers to the extracellular domains from SARS-CoV, SARS-CoV-2, MERS-CoV, and HKU1 containing the homologous 2P mutations (residues 986 and 987 (Wrapp et al., 2020) in SARS-CoV-2) and C-terminally fused to a foldon domain, unless otherwise noted. To generate S2-specific antibodies, three Balb/c mice were immunized with stabilized MERS-CoV S2 protein and boosted four weeks later, resulting in strong serum antibody with titers detectable at >1:10,000 dilution. The MERS-CoV S2 protein includes amino acid residues 763-1291 of MERS-S-2P of the MERS-CoV spike protein with a C-terminal T4 phage fibritin (foldon) domain that assembles into a pre-fusion trimer. To generate immune antibody libraries, total mRNA was isolated from the mouse spleens and reverse transcribed; the antibody variable regions were then amplified (Krebber et al., 1997) and inserted into the pMopac24 M13 bacteriophage display vector (Hayhurst et al., 2003) to express pIII-scFv-c-myc tag fusion proteins. The phage display library (−3.1×10⁸ individual clones) was subjected to four rounds of panning with immobilized antigen: anti-c-myc antibody to deplete truncated or non-expressing clones, then MERS-CoV S2, and finally SARS-CoV-2 spike at a high concentration followed by moderate coating concentrations.

Phage clones isolated after rounds 3 and 4 were confirmed to bind MERS-CoV S2 and SARS-CoV-2 spike by ELISA and evaluated for binding to uncoated plates and the foldon domain. While no clones exhibited plate or milk binding, ˜85% of round 3 and 4 clones bound the shared foldon domain fused to the RSV F protein (McLellan et al., 2013) (FIG. 9). One of these foldon binders, 3E11, was carried forward as a control antibody. Three unique clone families were identified from the remaining clones specific to the spike protein: 3A3, 4A5, and 4H2. Antibody 3A3 and close relatives appeared in round 3 and were enriched to ˜10% of the population after round 4, while 4A5 and 4H2 were unique sequences isolated after round 4 of panning.

### Example 2—Cross-Reactive Antibodies Bind the Spike S2 Domain with High Affinity and Specificity

After expression in ExpiCHO cells and purification of 3A3, 4A5, 4H2, and 3E11 as full-length antibodies with human Fc and kappa domains, the antibodies were biophysically characterized to measure spike binding kinetics by biolayer interferometry (BLI), thermal unfolding by thermal shift, polydispersity by size exclusion chromatography (SEC), and purity by SDS-PAGE (Tables 9 and 10, FIGS. 10 and 11). The antibodies all appeared as expected for intact immunoglobulins, with 4A5 exhibiting a slightly delayed SEC retention volume. All four antibodies exhibited low- to mid-nanomolar equilibrium affinities for the SARS-CoV-2 spike (Wrapp et al., 2020) as measured by BLI. Additional measurements with SARS-CoV-2 HexaPro, an ultra-stable SARS-CoV-2 spike variant with six proline substitutions relative to wild-type spike (Hsieh et al., 2020), confirmed these affinities. The binding of 3A3 Fab to the isolated S2 domain of SARS-CoV-2 HexaPro revealed similar ˜2.5 nM K_{d} values by both SPR and BLI. The on- and off-rate constants for HexaPro S2 binding in the absence of S1 were ˜2×10⁶ M⁻¹s⁻¹ and 5×10⁻³ s⁻¹, respectively (FIG. 11). Although 4A5 and 4H2 had equilibrium affinities in the range of 3A3, their relatively fast off-rates (FIG. 11) were reflected in lower ELISA EC₅₀ values (FIG. 1).

To assess the phylogenetic range of spike recognition by these three antibodies, binding to SARS-CoV, SARS-CoV-2, MERS-CoV, and HKU1 as well as SARS-CoV-2 HexaPro spikes, and RSV F-foldon control was assessed by ELISA (FIG. 1A) and validated with BLI K_{d} measurements (Tables 6 and 7, FIG. 11D). Antibodies 3A3, 4A5, and 4H2 were able to bind the SARS-CoV spike, MERS-CoV spike, and MERS-CoV S2 domain, but exhibited significantly reduced binding to HKU1 spike and no detectable binding to RSV F-foldon. Specifically, 3A3 exhibited an affinity of 20 nM for SARS-CoV and 23 nM for MERS-CoV intact spikes. The foldon binding antibody 3E11 showed relatively high binding affinity ˜3-11 nM across all spikes and the RSV F-foldon as expected since these proteins all include foldon domains for stabilization.

TABLE 9
Binding affinities for SARS-2 spike variants and antibody thermal stabilities*

| | | BLI | | BLI | | |
|---|---|---|---|---|---|---|
| | SPR | HexaPro | BLI | aglyc | BLI | |
| | HexaPro S2 | S2 | HexaPro | HexaPro | 2P | |
| | Fab K_{d} | Fab K_{d} | IgG K_{d} | IgG K_{d} | IgG K_{d} | IgG1 Tₘ |
| antibody | (nM) | (nM)† | (nM)† | (nM)† | (nM)† | (° C.) |
| 3A3 | 2.2 | 3.2 ± 0.3 | 12.0 ± 0.3 | 8.4 ± 0.3 | 2.5 ± 0.1 | 76.9 ± 0.8 |
| 4A5 | ND | 11.0 ± 0.1 | 3.2 ± 0.3 | 10 ± 2 | 14 ± 2 | 77.6 ± 0.3 |
| 4H2 | ND | 63 ± 7 | 6.4 ± 0.6 | 9 ± 2 | 26 ± 2 | 76.9 ± 0.1 |
| 3E11 | ND | 10 ± 1 | 6.7 ± 0.3 | ND | 5.0 ± 0.2 | 81.6 ± 0.2 |

*error is range of replicate measurements for K_{d} and standard deviation of triplicate measurements for the second transition melting temperature, Tₘ.
†IgG K_{d} values are equilibrium affinity of immobilized full-length IgGs on anti-Fc sensors capturing the indicated SARS-2 spike or spike domain.
ND = no data.

TABLE 10
Antibody binding affinities for spike variants†

| | Spike variant K_{d} (nM) | | | | | | |
|---|---|---|---|---|---|---|---|
| | | HexaPro | | HexaPro | | | MERS |
| | SARS-2 | S2 | HexaPro | aglyc | SARS-1 | MERS | S2 |
| 3A3 | 2.7 ± 0.3 | 3.2 ± 0.3 | 8 ± 3 | 7.9 ± 0.8 | 20 ± 2 | 23 ± 1.2 | 5.9 ± 0.6 |
| 4A5 | 17 ± 4 | 11.0 ± 0.1 | 4 ± 1 | 10 ± 2 | 4 ± 0.9 | ND | ND |
| 4H2 | 29 ± 4 | 63 ± 7 | 5.8 ± 0.9 | 9 ± 2 | 20 ± 2 | ND | ND |
| 3E11 | 5.5 ± 0.7 | 8 ± 4 | 3.5 ± 0.6 | 11 ± 2 | 5.7 ± 0.5 | ND | ND |

†K_{d} values represent the equilibrium affinity for full-length IgGs immobilized on anti-Fc tips capturing the indicated SARS-2 spike or spike domain, as measured by BLI with standard error shown. All fits had R² > 0.90.
ND = no data.

### Example 3—Antibody 3A3 Binds a Conformationally Sensitive S2 Epitope

To further investigate the basis for spike protein recognition, the SARS-CoV-2, SARS-CoV-2 HexaPro, MERS-CoV, and HKU1 spikes were fully denatured and reduced and subjected to western blotting. Quadruplicate blots were incubated with each full-length antibody and binding to spike polypeptide was detected with anti-human-Fc-HRP and chemiluminescent peroxidase substrate (FIG. 1B). Antibodies 4A5, 4H2, and 3E11 detected linearized SARS-CoV-2, SARS-CoV-2 HexaPro, and MERS-CoV spike proteins, with undetectable binding to HKU1 spike. In contrast, 3A3 did not bind any spike protein in western blot, indicating that the 3A3 antibody binds a purely conformational epitope, while the other three antibodies recognize epitopes with significant linear components.

To better understand the 3A3 conformational epitope, 3A3 binding to fresh versus stressed SARS-CoV-2 spike was evaluated by ELISA (FIGS. 1C and 1D). Soluble SARS-CoV-2 spike has low stability, with lower production yields than SARS-1 and MERS, despite sharing the homologous “2P” proline mutations (˜0.5 mg/L versus ˜6 mg/L and ˜20 mg/L, respectively) (Wrapp et al., 2020; Pallesen et al., 2017; Hsieh et al., 2020), and is sensitive to freeze-thaw stresses. By contrast, SARS-CoV-2 HexaPro includes four additional proline substitutions and exhibits resistance to freeze-thaw stresses (Hsieh et al., 2020). Spike was stressed by three freeze/thaw cycles, which produced SDS-PAGE-detectable aggregates in the SARS-CoV-2 spike but not SARS-CoV-2 HexaPro (FIG. 13) and captured on antibody-coated ELISA plates. Antibodies 4A5, 4H2, and 3E11 bound fresh and stressed SARS-CoV-2 spike proteins similarly, with 4A5 and 4H2 binding stressed spike slightly better (FIG. 1D). However, 3A3 bound stressed 2P spike with a ˜150-fold worse EC₅₀, while binding for stressed SARS-CoV-2 HexaPro was unaffected, indicating a reduced capacity to bind misfolded and/or aggregated spike.

### Example 4—Antibody 3A3 Neutralizes Spike in In Vitro Cellular Fusion and Pseudovirus Infection Assays

To investigate the abilities of 3A3, 4A5, and 4H2 to impact spike function, a mammalian cell fusion assay was employed (FIG. 2). A CHO cell line expressing wild-type SARS-CoV-2 spike and GFP was incubated with ACE2-expressing HEK 293 cells dyed with the red fluorescent Cell Trace Far Red stain. After 24 hours, large syncytia formed with green CHO cell fluorescence overlapping ˜70% of red HEK 293 cell fluorescence, indicating fusion of the CHO and HEK 293 membranes in the presence of no antibody (FIG. 2A) or 100 μg/mL (670 nM) irrelevant human IgG1 (FIG. 2D), 4A5, or 4H2 antibodies (FIG. 14). Minimal fluorescence colocalization occurred if either the CHO cells did not express SARS-CoV-2 spike (FIG. 2B) or the HEK 293 cells did not express ACE2 (FIG. 2C). Incubation with 100 μg/mL (670 nM) (FIG. 2G) or 10 μg/mL (67 nM) (FIG. 2F) of 3A3 significantly reduced colocalization to ˜50% (p<0.0001; FIG. 2H). While 1 μg/mL (6.7 nM) 3A3 had minimal impact on fluorescence colocalization (FIG. 2E), analysis of average cell size showed significantly reduced syncytia size in the presence of 1 μg/mL (6.7 nM) 3A3 (FIG. 2I).

Next, 3A3 neutralization was assessed in an in vitro pseudovirus neutralization assay. Antibody 3A3 or isotype control was preincubated for 1 hour with pseudotyped VSV virus expressing either wild-type or D614G SARS-2 spike, both without stabilizing modifications, then added to HEK293 cells expressing ACE2. The pseudovirus induced chemiluminescence in infected cells and the extent of infection was tracked over 72 hours (FIG. 3A). With one hour of preincubation, antibody 3A3 blocked infection of wild-type spike pseudovirus with an IC₅₀ of ˜26 μg/mL (173 nM), and D614G spike pseudovirus with an IC₅₀ of ˜2.1 μg/mL (14 nM).

In addition, 3A3 neutralization was assessed in another in vitro pseudovirus neutralization assay. Antibody 3A3, potently neutralizing antibody S309 (Pinto et al., 2020), or an isotype control antibody was preincubated for 1 hour with pseudotyped lentivirus expressing either SARS-CoV spike, wild-type SARS-CoV-2 spike, SARS-CoV-2 D614G spike, or SARS-CoV-2 B.1.1.7 (variant Alpha) spike, all without stabilizing modifications, then added to HEK293 cells expressing ACE2. The pseudovirus induced luciferase expression in infected cells and the extent of infection was tracked over 72 hours (FIG. 3B). With one hour of preincubation, the positive control S309 had an IC₅₀ of −1 nM against SARS-CoV and SARS-CoV-2 spike, similar to values found with MLV (Pinto et al., 2020) pseudovirus. Antibody S309 was less potent against the B.1.1.7 pseudotyped-virus than wild-type or D614G, as previously reported (Wang et al., 2021), while the isotype control had no effect on any pseudoviruses. By contrast, 3A3 blocked infection of wild-type spike pseudovirus with an IC₅₀ of 212 nM, D614G spike pseudovirus with an IC₅₀ of 94 nM, and B.1.1.7 spike pseudovirus with an IC₅₀ of 119 nM (Table 11). SARS-CoV spike pseudotyped virus was neutralized by 3A3 with an IC₅₀ of 188 nM. Consistent with pseudovirus neutralization, 3A3 bound D614G spike displayed on mammalian cells more readily than wild-type spike by flow cytometry (FIG. 6F).

TABLE 11
Antibody neutralization of pseudovirus IC₅₀ values

| | Pseudovirus IC₅₀ | | | | |
|---|---|---|---|---|---|
| | (95% confidence interval), nM | | | | |
| | SARS-2 | D614G | B.1.1.7 | SARS-1 | VSV-G |
| 3A3 | 212 | 94 | 119 | 188 | 924 |
| | (167-273) | (73-122) | (103-138) | (127-283) | (668-1380) |
| S309 | 1.0 | 0.8 | 48 | 0.5 | >5000 |
| | (0.8-1.2) | (0.6-0.9) | (39-59) | (0.5-0.6) | |
| Isotype | >5000 | >5000 | >5000 | >5000 | >5000 |
| control | | | | | |

### Example 5—the Neutralizing 3A3 Epitope is Located on the S2 Hinge

To identify the specific epitope recognized by 3A3, hydrogen-deuterium exchange mass spectrometry (HDX-MS) was used. Deuterium uptake of the SARS-CoV-2 HexaPro spike alone, as well as bound by the 3A3 IgG or 3A3 Fab, was measured (Table 8). Complexes were formed with excess antibodies such that SARS-CoV-2 HexaPro spike was ˜90% bound in both cases. 192 unmodified peptides were tracked through the deuteration time course (101, 10², 10′, and 104 s), covering over half of the protein sequence (FIG. 15). Glycosylated peptides were not searched for, as de-glycosylation had a small effect on 3A3 affinity (Tables 9 and 10). Analysis of the raw deuterium uptake in the SARS-2 HexaPro spike alone shows maintenance of the trimer during the HDX reaction. There was relatively low deuterium uptake in the helix at the center of the trimer, and high deuterium uptake in the HR1 helix at the surface of the trimer (FIG. 16).

Antibody epitopes were identified by looking at the difference in deuterium uptake between SARS-CoV-2 HexaPro spike in the free and antibody-bound states. A significant difference was defined as having a change in deuterium uptake greater than 0.2 Da with a p-value less than 0.01 (FIGS. 4A and 4B). Binding of 3A3 IgG caused a significant decrease to occur in 12 peptides that redundantly span residues 980 to 1006 of the SARS-CoV-2 HexaPro spike (FIGS. 4C and 17A). These peptides have reduced deuterium uptake with 3A3 IgG at several timepoints during the exchange reaction. An identical result was observed when the 3A3 Fab was used in place of the IgG, consistent with identical binding (FIGS. 4B, 4C, and 17B). In contrast to 3A3 IgG and Fab, similar experiments with 4A5 and 4H2 antibodies showed no difference in deuterium uptake upon antibody addition (FIGS. 18A-18C). The epitopes recognized by 4A5 and 4H2 are clearly distinct from 3A3 and possibly lie in regions where peptides were lacking. Taken altogether, these data clearly suggest that the 3A3 epitope lies within residues 980 to 1006 of the SARS-CoV-2 HexaPro spike.

Mapping the difference in deuterium uptake between free and 3A3-bound states onto the structure localized the epitope to the apex of the S2 domain, distal to the viral envelope (FIG. 4D). It covers the end of the HR1 helix, the two stabilizing proline mutations (residues 986 and 987) and the beginning of the C_{H} helix. This region is highly conserved in both sequence and structure across all β-coronaviruses known to infect humans (FIGS. 6B-6E). The RMSD of Cu atoms ranges from 0.6 Å for HKU1 to 3.1 Å for MERS-CoV. To confirm the epitope and show its validity to varied spike proteins, BLI was used to assess 3A3 binding to the MERS-CoV S2 domain, either wild-type or with the apex deleted. 3A3 was able to bind the MERS-CoV S2 domain, but lost binding when the apex was deleted (FIG. 19). This confirms that 3A3 directly binds the apex of the S2 domain of the spike proteins from varied coronaviruses.

Fourteen of the solvent exposed residues within the 3A3 epitope identified by HDX-MS were altered to assess the impact on 3A3 binding. Three, S982A (present in the B.1.1.7 SARS-CoV-2 Alpha variant), L984V and R995A improved 3A3 binding by ELISA, while five (D985L, E988Q or I, D994A, L1001A and Q1002A) significantly reduced 3A3 binding (FIGS. 6G, 6H, and 21). The most substantial impact was the near ablation of binding by the substitutions at positions D985 and E988. These residues form a negatively charged patch adjacent to the 2P changes, P986 and P987. Since E988Q is present in the spike proteins of the α-coronaviruses NL63 and 229E, these data suggest 3A3 neutralization may be limited to β-coronaviruses. In addition to the clear role of residues near the S2 hinge, residues D994, L1001 and Q1002 lie deeper in the S2 core and are hidden in prefusion trimer structures (Wrapp et al., 2020; Hsieh et al., 2020), yet appear to contribute to 3A3 spike recognition. Substitutions that improve binding are at the protomer interfaces and may destabilize the closed trimer conformation.

### Example 6—HD Results, RBD Position Influences Access to the 3A3 Epitope

Mapping the HDX data onto the SARS-2 HexaPro spike structure (FIG. 3); highlights: This 3A3 epitope spanning residues 986-1006 is located at the apex of the S2 domain, distal to the viral membrane. The location of the 3A3 epitope ultimately suggests its accessibility is regulated by the position of the RBDs in the S1 domain. The epitope is completely hidden in closed spike with all three RBDs in the down position, and becomes increasingly exposed as one, two, or three of the RBDs adopt an up position or bind the ACE2 receptor (FIG. 5). Consistent with this, 3A3 did not bind a SARS-2 HexaPro spike that was locked into the closed conformation with disulfide bonds, although the control 2-4 antibody bound well (FIG. 6A).

3A3 binding to several engineered spike proteins was evaluated to provide biochemical support for the identified epitope. First, an apex-less MERS S2 domain with amino acids with residues 811-824 replaced with GGSGGS and residues 1042-1073 replaced with a flexible linker was evaluated for binding to 3A3 by BLI. In the MERS spike, the 3A3 epitope is amino acids 1052-1080, so much of it is removed in apex-less MERS S2. While 3A3 was able to bind MERS S2, it exhibited no binding to the apex-less variant (FIG. 19). Second, since this epitope is completely hidden in closed spike, but becomes increasingly exposed in structures with one, two, or three RBDs up (FIG. 5), it was hypothesized that 3A3 would be unable to bind a HexaPro variant locked in the closed state due to an introduced di-sulfide bond through S383C-D985C substitutions. Binding by BLI confirmed that 3A3 cannot bind this closed SARS-2 HexaPro spike, while mAb 2-4 which is specific for RBD-down spike, bound both (FIG. 6A).

Since the 3A3 epitope sequence and structure are highly conserved across coronaviruses known to infect humans, with 41% identity and 62% sequence similarity (FIG. 6B) and rmsd values ranging from 0.8 Å (HKU1) to 3.5 Å (MERS) versus to SARS-2 structure (FIG. 6D), it was hypothesized that the major determinant in 3A3 binding is epitope access. The inventors therefore evaluated 3A3 binding to naturally occurring spike variants with different open versus closed propensities, namely the wild-type versus D614G SARS-CoV-2 variants, which are observed in the closed state in ˜90% versus 5% of particles, respectively (Cai et al., 2020; Yurkovetskiy et al., 2020). Minimal 3A3 binding to wild-type spike (without stabilizing mutations) expressed on the surface of mammalian cells was observed after one hour of staining. By contrast, 3A3 strongly bound surface-expressed D614G spike (also without 2P stabilization) (FIG. 6F), consistent with this variant providing more frequent access to the 3A3 epitope.

In the same flow cytometry assay, antibody CR3022 (Yuan et al., 2020), a rare SARS-CoV/SARS-CoV-2 cross-reactive RBD-binding antibody that binds a cryptic epitope requiring RBDs up (Yuan et al., 2020), exhibited reduced binding to D614G than wild-type spike (FIG. 6F), suggesting it is less relevant than 3A3 for binding a range of circulating SARS-2 strains. Finally, to evaluate the possibility that 3A3 binds immediately following S1 dissociation, capturing the S2 domain in a transient but still prefusion state, binding of 3A3-bound HexaPro to the RBD-binding antibody S309 (Pinto et al., 2020) was evaluated using BLI. Since strong S309 binding was observed, 3A3 can bind when at least some S1 monomers are still present (FIG. 20).

### Example 7—S2 Opening Provides Access to the 3A3 Epitope

The HDX epitope mapping and point mutagenesis data indicate that 3A3 binds near the trimer interface of S2, which is poorly accessible in published spike ectodomain structures. Costello et al. carried out an independent HDX experiment which confirmed the 3A3 epitope and revealed that spike undergoes reversible protomer separation, exposing the S2 core and the 3A3 epitope (Costello et al., 2021). With this knowledge in hand, we revisited our spectra and observed bimodal spectra. These spectra were less obvious in the present work because of deuterium and peptide length. Moreover, the HP used in these experiments would have a small amounts of the S2-open conformation. The S2-open state is similar to those transiently found in other viral fusion proteins such as the respiratory syncytial virus class I fusion protein, RSV F (Gilman et al., 2019), and the vesicular stomatitis virus class III fusion protein, VSV-G (Abou-Hamdan et al., 2018; Albertini et al., 2012). HDX carried out under conditions that favor the S2-open or S2-closed conformations demonstrated that 3A3 selectively binds the S2-open state. Consistent with this, 3A3 did not bind a SARS-CoV-2 HexaPro spike that was locked into the closed conformation by engineered disulfide bonds (Henderson et al., 2020) (FIG. 12, bottom) although this constrained spike was recognized by the control 2-4 antibody that binds the RBDs in the down state (FIG. 12, top).

Analysis of 3A3 binding to various SARS-CoV-2 and SARS-CoV-2 HexaPro spikes with identical 3A3 epitope sequences resulted in inconsistent on-rates and K_{d}values by BLI, likely reflecting epitope accessibility as opposed to the affinity of the epitope/paratope interaction (FIGS. 7A and 22). Preincubating spike at 4° C. for over one week or 37° C. for 24 hours biases spike into the S2-open versus S2-closed conformation, respectively, and correspondingly alters the fraction of spikes with accessible epitope and the apparent 3A3 binding kinetics. The SARS-CoV-2 spike is slightly unstable at 37° C., which alters association of the control antibody S309 (Pinto et al., 2020) for spike by ˜2-fold (BLI data; FIG. 7A) and EC₅₀ by ˜3-fold (ELISA data; FIG. 23) relative to spike incubated at 4° C. However, 3A3-SARS-CoV-2 spike binding was markedly reduced after 37° C. incubation (˜5.5-fold reduced on-rate constant by BLI and ˜30-fold increase in ELISA EC₅₀). This was also evident in 3A3 binding to SARS-CoV-2 HexaPro after 4° C. or 37° C. incubation (FIG. 7A). In general, SARS-CoV-2 spike had faster 3A3 on-rate kinetics than SARS-CoV-2 HexaPro, perhaps due to the bias toward the S2-open state in SARS-CoV-2 spike relative to SARS-CoV-2 HexaPro (Costello et al., 2021) (FIG. 23).

Two additional SARS-CoV-2 HexaPro variants were produced to confirm binding to the S2-open spike state. The E1031R substitution was introduced into SARS-CoV-2 HexaPro, which is outside of the 3A3 epitope but disrupts an electrostatic interaction between E1031 and R1039 on adjacent protomers deep in the S2 base. When evaluated by HDX-MS, this substitution promoted the formation of the S2-open state relative to unmodified HexaPro (FIG. 24A). To generate a monomeric HexaPro (monoHP) in which the 3A3 epitope is always exposed, a protease cleavage site was introduced between the C-terminus of the SARS-CoV-2 HexaPro spike and the foldon domain. After 5 days of 4° C. incubation, the foldon trimerization motif was cleaved and monoHP isolated by SEC (FIG. 24B). When either E1031R or monoHP was assessed for 3A3 binding by BLI, the on-rate was faster than that of fresh, 4° C. stored, or 37° C. stored unmodified SARS-CoV-2 HexaPro, likely due to improved epitope accessibility (FIG. 7A).

Although ACE2 can bind spike in both the S2-open and S2-closed spike states due to rapid sampling of the RBD up/down positions, SARS-CoV-2 HexaPro spike shifts toward the S2-open state upon ACE2 binding (Costello et al., 2021). It is possible that in the context of unmodified spike on the viral envelope, as in the mammalian cell fusion and pseudovirus neutralization assays (FIG. 3), 3A3 primarily binds when the spike attaches to ACE2. BLI was used to confirm that spike can simultaneously bind ACE2 and 3A3. Immobilized 3A3 captured SARS-CoV-2 HexaPro and then soluble ACE2 (FIG. 7B, right), while mAb 2-4, which binds the RBD down state across protomers, could bind SARS-CoV-2 HexaPro but could not simultaneously bind ACE2 (FIG. 7B, left).

### Example 8—Humanization of Antibody 3A3

Humanized 3A3 light and heavy chain sequences were designed based on the mu3A3VL and VH sequences isolated from the phage library of the mouse immunization library. The following humanization protocols were used:

(a) CDR grafting [according to Jones et al. (1986) “Replacing the complementarity-determining regions in a human antibody with those from a mouse” Nature 321:522-525; Verhoeyen et al. (1988) “Reshaping human antibodies: grafting an antilysozyme activity” Science 239:1534-1536]. The CDRs, as defined by Kabat et al. (1991) “Sequences of Proteins of Immunological Interest” 5th ed. US Department of Health and Human Services, Public Health Service, National Institutes of Health (NIH Publication No 91-3242), were grafted onto a human scaffold. The critical framework residues, e.g., the buried residues which probably play a significant role in maintaining the combining site structure, the residues which have been found to be involved in the VL:VH contact, and the non-CDR residues which have been found to be involved in the contact with antigen (see, for example, Padlan (1994) “Anatomy of the Antibody Molecule” Mol Immunol 1994; 31(3):169-217; Narciso et al. (2012) “Anatomy of the antibody molecule: a continuing analysis based on high-resolution crystallographic structures” Phil Sci Letts 5(1):63-89), were preserved. The resulting heavy chain and light chain are termed “cdr3A3VH” and “cdr3A3VL”, respectively, which are represented by SEQ ID NOS: 70 and 71, respectively.

(b) Grafting of abbreviated CDRs [according to Padlan et al. (1995) “Identification of specificity-determining residues in antibodies” FASEB J 9:133-139]. The resulting heavy chain is termed “abb3A3VH”, which is represented by SEQ ID NO: 74.

(c) SDR-transfer [according to Padlan et al. (1995) “Identification of specificity-determining residues in antibodies” FASEB J 9:133-139]. The resulting heavy chain and light chain are termed “sdr3A3VH” and “sdr3A3VL”, respectively, which are represented by SEQ ID NOS: 72 and 73, respectively.

(d) Veneering [according to Padlan (1991) “A possible procedure for reducing the immunogenicity of antibody variable domains while preserving their ligand-binding properties” Mol Immunol 28:489-498]. The resulting heavy chain and light chain are termed “ven3A3VH” and “ven3A3VL”, respectively, which are represented by SEQ ID NOS: 68 and 69, respectively.

The humanized sequences (see Table 6) were cloned into full length human antibody IgG1 and IgK plasmids identical to those used for chimeric 3A3 (mouse V regions, human C regions) expression. Each heavy chain made by the different protocols was paired with each of the light chains. After expression in ExpiCHO cells, ELISAs were used to measure both expression level (coat with anti-huFc, detect with anti-huK-HRP) and binding to HexaPro (coat with HexaPro, detect with anti-huFc-HRP) compared to purified ch3A3 (FIGS. 25-27).

Antibodies were purified by protein A binding on an FPLC and concentrations determined based on their calculated extinction coefficients and molecular weights. Purified antibodies were then compared to purified ch3A3 on a HexaPro ELISA. The antibodies performed similarly, however, the only antibody without the venVH sequence had the highest EC₅₀ (weakest binding) (FIG. 28). Antibodies with the cdrVL, sdrVL, or venVL and venVH performed equivalently to ch3A3 (FIG. 28).

### Example 9—Affinity Maturation of Humanized Antibody 3A3

Though 3A3 has an affinity for SARS-CoV-2 S2P and HexaPro stabilized spikes in the 2-3 nM range, it's binding to spike with the stabilizing 986P and 987P reverted to 986K and 987V is ˜200-fold worse. The relatively low IC₅₀ of neutralization of 3A3 in pseudovirus neutralization assays is reflective of this loss of binding for the authentic spike sequence.

To address this, site directed and error prone (random) libraries of the V_{L} and V_{H} regions were made and yeast Fab display used to screen these libraries. The libraries were screened for binding to soluble SARS-CoV-2 spike protein variant 4PDS, with F817P, A892P, A899P, A942P, 986K, 987V, and a disulfide bond formed by the mutations Q965C and S1003C (FIG. 29). Binding to a spike variant 4P (F817P, A892P, A899P, A942P), which has the reverted prolines at 986 and 987, but lacks the added disulfide, was also screened.

A total of 32 clones were analyzed by flow cytometry with 20 nM 4PDS-AF647 staining. Of these, five antibodies (AM3A3-1, AM3A3-2, AM3A3-3, AM3A3-4, AM3A3-5) were sequenced and cloned for mammalian expression. The heavy and light chains for AM3A3-1 (LC only), AM3A3-2 (HC only), AM3A3-3, AM3A3-4, and AM3A3-5 were combinatorially screened with the other heavy and light chains as well as unmodified hu3A3 heavy and light chains (wild-type or “WT”; ven3A3) for small scale mammalian expression. Those with AM3A3-1 light chain did not express at all, but all others expressed with approximately similar yields. An ELISA capturing antibody from mammalian expression media indicates that AM3A3-4 heavy and light chains and AM3A3-2 heavy chains do not significantly improve binding to 4PDS. However, AM3A3-3 and AM3A3-5 and combinations of the heavy and light chains with each other or WT yield improvements in binding to 4PDS (FIG. 30).

Two antibody sequences, AM3A3-3 and AM3A3-5 (Table 7), were obtained that dramatically improve binding to 4PDS and 4P while retaining binding to HexaPro. The V_{L} and V_{H} regions of these sequences may be swapped (e.g., V_{L}5/V_{H}3) and maintain this binding profile.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Abdelrahman et al., Comparative Review of SARS-CoV-2, SARS-CoV, MERS-CoV, and Influenza A Respiratory Viruses. Front Immunol 11, 552909 (2020).
Abou-Hamdan et al., Monomeric Intermediates Formed by Vesiculovirus Glycoprotein during Its Low-pH-induced Structural Transition. J Mol Biol 430, 1685-1695 (2018).
Albertini et al., Characterization of monomeric intermediates during VSV glycoprotein structural transition. PLoS Pathog 8, e1002556 (2012).
Andreano et al., SARS-CoV-2 escape in vitro from a highly neutralizing COVID-19 convalescent plasma. bioRxiv (2020).
Bangaru et al., A Site of Vulnerability on the Influenza Virus Hemagglutinin Head Domain Trimer Interface. Cell 177, 1136-1152 e1118, doi:10.1016/j.cell.2019.04.011 (2019).
Barnes et al., SARS-CoV-2 neutralizing antibody structures inform therapeutic strategies. Nature 588, 682-687 (2020).
Bolte & Cordelieres, A guided tour into subcellular colocalization analysis in light microscopy. J Microsc 224, 213-232 (2006).
Cai et al., Distinct conformational states of SARS-CoV-2 spike protein. Science 369, 1586-1592 (2020).
Channappanavar et al., Protective Effect of Intranasal Regimens Containing Peptidic Middle East Respiratory Syndrome Coronavirus Fusion Inhibitor Against MERS-CoV Infection. J Infect Dis 212, 1894-1903 (2015).
Chen et al., Human monoclonal antibodies block the binding of SARS-CoV-2 spike protein to angiotensin converting enzyme 2 receptor. Cell Mol Immunol 17, 647-649 (2020).
Chi et al., A neutralizing human antibody binds to the N-terminal domain of the Spike protein of SARS-CoV-2. Science 369, 650-655 (2020).
Corti et al., Tackling influenza with broadly neutralizing antibodies. Curr Opin Virol 24, 60-69 (2017).
Costello, S. M. et al. The SARS-CoV-2 spike reversibly samples an open-trimer conformation exposing novel epitopes. bioRxiv (2021).
Crank et al., A proof of concept for structure-based vaccine design targeting RSV in humans. Science 365, 505-509 (2019).
Crawford et al., Protocol and Reagents for Pseudotyping Lentiviral Particles with SARS-CoV-2 Spike Protein for Neutralization Assays. Viruses 12, doi:10.3390/v12050513 (2020).
Davies et al., Estimated transmissibility and impact of SARS-CoV-2 lineage B.1.1.7 in England. Science 372, doi:10.1126/science.abg3055 (2021).
DeLano. (DeLano Scientific, San Carlos, C A, 2002).
Elshabrawy et al., Human monoclonal antibodies against highly conserved HR1 and HR2 domains of the SARS-CoV spike protein are more broadly neutralizing. PLoS One 7, e50366 (2012).
Gilman et al., Transient opening of trimeric prefusion RSV F proteins. Nat Commun 10, 2105 (2019).
Goike et al., Synthetic repertoires derived from convalescent COVID-19 patients enable discovery of SARS-CoV-2 neutralizing antibodies and a novel quaternary binding modality. bioRxiv, doi:10.1101/2021.04.07.438849 (2021).
Greaney et al., Mapping mutations to the SARS-CoV-2 RBD that escape binding by different classes of antibodies. Nat Commun 12, 4196 (2021).
Hayhurst et al., Isolation and expression of recombinant antibody fragments to the biological warfare pathogen Brucella melitensis. J Immunol Methods 276, 185-196 (2003).
Henderson et al., Controlling the SARS-CoV-2 spike glycoprotein conformation. Nat Struct Mol Biol 27, 925-933 (2020).
Hsieh et al., Structure-based design of prefusion-stabilized SARS-CoV-2 spikes. Science 369, 1501-1505 (2020).
Impagliazzo et al., A stable trimeric influenza hemagglutinin stem as a broadly protective immunogen. Science 349, 1301-1306 (2015).
Jones et al., The neutralizing antibody, LY-CoV555, protects against SARS-CoV-2 infection in nonhuman primates. Sci Transl Med 13, doi:10.1126/scitranslmed.abf1906 (2021).
Kim, et al., Mechanism of membrane fusion induced by vesicular stomatitis virus G protein. Proc Natl Acad Sci USA 114, E28-E36, doi:10.1073/pnas.1618883114 (2017).
Kirchdoerfer et al., Stabilized coronavirus spikes are resistant to conformational changes induced by receptor recognition or proteolysis. Sci Rep 8, 15701 (2018).
Krebber et al., Reliable cloning of functional antibody variable domains from hybridomas and spleen cell repertoires employing a reengineered phage display system. J Immunol Methods 201, 35-55 (1997).
Lee et al., Molecular-level analysis of the serum antibody repertoire in young adults before and after seasonal influenza vaccination. Nat Med 22, 1456-1464, doi:10.1038/nm.4224 (2016).
Lip et al., Monoclonal antibodies targeting the HR2 domain and the region immediately upstream of the HR2 of the S protein neutralize in vitro infection of severe acute respiratory syndrome coronavirus. J Virol 80, 941-950 (2006).
Liu et al., Cross-Neutralization of a SARS-CoV-2 Antibody to a Functionally Conserved Site Is Mediated by Avidity. Immunity 53, 1272-1280 e1275, doi:10.1016/j.immuni.2020.10.023 (2020).
Liu et al., Potent neutralizing antibodies against multiple epitopes on SARS-CoV-2 spike. Nature 584, 450-456 (2020).
Lu et al., Structure-based discovery of Middle East respiratory syndrome coronavirus fusion inhibitor. Nature communications 5, 3067 (2014).
Lu et al., Generation of Chicken IgY against SARS-COV-2 Spike Protein and Epitope Mapping. J Immunol Res 2020, U.S. Pat. No. 9,465,398 (2020).
Lv et al., Cross-reactive Antibody Response between SARS-CoV-2 and SARS-CoV Infections. Cell Rep 31, 107725 (2020).
McLellan et al., Structure-based design of a fusion glycoprotein vaccine for respiratory syncytial virus. Science 342, 592-598 (2013).
Montefiori et al., The high-affinity immunoglobulin receptor FcgammaRI potentiates HIV-1 neutralization via antibodies against the gp41 N-heptad repeat. Proc Natl Acad Sci USA 118, doi:10.1073/pnas.2018027118 (2021).
Ng et al., Preexisting and de novo humoral immunity to SARS-CoV-2 in humans. Science, 370, 1339-1343 (2020).
Nguyen et al., A cocktail of humanized anti-pertussis toxin antibodies limits disease in murine and baboon models of whooping cough. Sci Transl Med 7, 316ra195 (2015).
Nguyen et al., Identification of high affinity HER2 binding antibodies using CHO Fab surface display. Protein Eng Des Sel 31, 91-101 (2018).
Pallesen et al., Immunogenicity and structures of a rationally designed prefusion MERS-CoV spike antigen. Proc Natl Acad Sci USA 114, E7348-E7357 (2017).
Pinto et al., Cross-neutralization of SARS-CoV-2 by a human monoclonal SARS-CoV antibody. Nature 583, 290-295 (2020).
Pinto et al., A human antibody that broadly neutralizes betacoronaviruses protects against SARS-CoV-2 by blocking the fusion machinery. bioRxiv (2021).
Plante et al., Spike mutation D614G alters SARS-CoV-2 fitness. Nature 592, 116-121 (2021).
Poh et al., Two linear epitopes on the SARS-CoV-2 spike protein that elicit neutralising antibodies in COVID-19 patients. Nat Commun 11, 2806 (2020).
Rappazzo et al., Broad and potent activity against SARS-like viruses by an engineered human monoclonal antibody. Science, doi:10.1126/science.abf4830 (2021).
Raybould et al., CoV-AbDab: the coronavirus antibody database. Bioinformatics 37, 734-735, doi:10.1093/bioinformatics/btaa739 (2021).
Renn et al., Fruitful Neutralizing Antibody Pipeline Brings Hope To Defeat SARS-Cov-2. Trends Pharmacol Sci 41, 815-829 (2020).
Rubio et al., A Derivative of the D5 Monoclonal Antibody That Targets the gp41 N-Heptad Repeat of HIV-1 with Broad Tier-2-Neutralizing Activity. J Virol 95, e0235020, doi:10.1128/JVI.02350-20 (2021).
Sauer et al., Structural basis for broad coronavirus neutralization. bioRxiv, 2020.12.29.424482 (2020).
Sauer, M. M. et al. Structural basis for broad coronavirus neutralization. Nat Struct Mol Biol 28, 478-486, doi:10.1038/s41594-021-00596-4 (2021).
Sliepen et al., Immunosilencing a highly immunogenic protein trimerization domain. J Biol Chem 290, 7436-7442 (2015).
Song et al., Cross-reactive serum and memory B cell responses to spike protein in SARS-CoV-2 and endemic coronavirus infection. bioRxiv, 2020.09.22.308965 (2020).
Song et al., Cross-reactive serum and memory B-cell responses to spike protein in SARS-CoV-2 and endemic coronavirus infection. Nat Commun 12, 2938, doi:10.1038/s41467-021-23074-3 (2021).
Starr et al., Prospective mapping of viral mutations that escape antibodies used to treat COVID-19. bioRxiv, 2020.11.30.405472 (2020).
Wagner et al., Human cytomegalovirus-specific T cell receptor engineered for high affinity and soluble expression using mammalian cell display. J Biol Chem, doi:10.1074/jbc.RA118.007187 (2019).
Wang et al., Importance of Neutralizing Monoclonal Antibodies Targeting Multiple Antigenic Sites on the Middle East Respiratory Syndrome Coronavirus Spike Glycoprotein To Avoid Neutralization Escape. J Virol 92, e02002-17 (2018).
Wang et al., Antibody resistance of SARS-CoV-2 variants B.1.351 and B.1.1.7. Nature 593, 130-135, (2021).
Wrapp et al., Cryo-EM structure of the 2019-nCoV spike in the prefusion conformation. Science 367, 1260-1263 (2020).
Wrapp et al., Structural Basis for Potent Neutralization of Betacoronaviruses by Single-Domain Camelid Antibodies. Cell 181, 1436-1441 (2020).
Wu et al., A noncompeting pair of human neutralizing antibodies block COVID-19 virus binding to its receptor ACE2. Science 368, 1274-1278, doi:10.1126/science.abc2241 (2020).
Yuan et al., Recognition of the SARS-CoV-2 receptor binding domain by neutralizing antibodies. Biochem Biophys Res Commun, doi: 10.1016/j.bbrc.2020.10.012 (2020).
Yuan et al., A highly conserved cryptic epitope in the receptor binding domains of SARS-CoV-2 and SARS-CoV. Science 368, 630-633 (2020).
Yurkovetskiy et al., Structural and Functional Analysis of the D614G SARS-CoV-2 Spike Protein Variant. Cell 183, 739-751 e738 (2020).
Wagner et al., Human cytomegalovirus-specific T cell receptor engineered for high affinity and soluble expression using mammalian cell display. J Biol Chem 294, 5790-5804 (2019).
Walls et al., Structure, Function, and Antigenicity of the SARS-CoV-2 Spike Glycoprotein. Cell 181, 281-292 e286 (2020).
Walsh et al., Safety and Immunogenicity of Two RNA-Based Covid-19 Vaccine Candidates. N Engl J Med 38, 2439-2450 (2020).
Watanabe et al., Vulnerabilities in coronavirus glycan shields despite extensive glycosylation. Nat Commun 11, 2688 (2020).
Zhang et al., The D614G mutation in the SARS-CoV-2 spike protein reduces S1 shedding and increases infectivity. bioRxiv 2020.06.12.148726 (2020).
Zhang et al., HD-eXplosion: Visualization of hydrogen-deuterium exchange data as chiclet and volcano plots with statistical filtering. Bioinformatics btaa892 (2020).
Zost et al., Rapid isolation and profiling of a diverse panel of human monoclonal antibodies targeting the SARS-CoV-2 spike protein. Nat Med 26, 1422-1427 (2020).

What is claimed is:

1. A monoclonal antibody or antibody fragment, wherein the antibody or antibody fragment comprises:
(a) a heavy chain variable region (VH) comprising VHCDR1, VHCDR2, and VHCDR3 amino acid sequences from the 3A3 antibody and a light chain variable region (VL) comprising VLCDR1, VLCDR2, and VLCDR3 amino acid sequences from the 3A3 antibody;
(b) a heavy chain variable region (VH) comprising VHCDR1, VHCDR2, and VHCDR3 amino acid sequences from the AM3A3-3 antibody and a light chain variable region (VL) comprising VLCDR1, VLCDR2, and VLCDR3 amino acid sequences from the AM3A3-3 antibody;
(c) a heavy chain variable region (VH) comprising VHCDR1, VHCDR2, and VHCDR3 amino acid sequences from the AM3A3-5 antibody and a light chain variable region (VL) comprising VLCDR1, VLCDR2, and VLCDR3 amino acid sequences from the AM3A3-5 antibody;
(d) a heavy chain variable region (VH) comprising VHCDR1, VHCDR2, and VHCDR3 amino acid sequences from the AM3A3-3 antibody and a light chain variable region (VL) comprising VLCDR1, VLCDR2, and VLCDR3 amino acid sequences from the AM3A3-5 antibody;
(e) a heavy chain variable region (VH) comprising VHCDR1, VHCDR2, and VHCDR3 amino acid sequences from the AM3A3-5 antibody and a light chain variable region (VL) comprising VLCDR1, VLCDR2, and VLCDR3 amino acid sequences from the AM3A3-3 antibody;
(f) a heavy chain variable region (VH) comprising VHCDR1, VHCDR2, and VHCDR3 amino acid sequences from the 4H2 antibody and a light chain variable region (VL) comprising VLCDR1, VLCDR2, and VLCDR3 amino acid sequences from the 4H2 antibody; or
(g) a heavy chain variable region (VH) comprising VHCDR1, VHCDR2, and VHCDR3 amino acid sequences from the 4A5 antibody and a light chain variable region (VL) comprising VLCDR1, VLCDR2, and VLCDR3 amino acid sequences from the 4A5 antibody.

2. The monoclonal antibody or antibody fragment of claim 1, wherein said antibody or antibody fragment comprises:
(a) a heavy chain variable region (VH) comprising VHCDR1, VHCDR2, and VHCDR3 amino acid sequences derived from SEQ ID NO: 55 and a light chain variable region (VL) comprising VLCDR1, VLCDR2, and VLCDR3 amino acid sequences derived from SEQ ID NO: 56;
(b) a heavy chain variable region (VH) comprising VHCDR1, VHCDR2, and VHCDR3 amino acid sequences derived from SEQ ID NO: 77 and a light chain variable region (VL) comprising VLCDR1, VLCDR2, and VLCDR3 amino acid sequences derived from SEQ ID NO: 78;
(c) a heavy chain variable region (VH) comprising VHCDR1, VHCDR2, and VHCDR3 amino acid sequences derived from SEQ ID NO: 79 and a light chain variable region (VL) comprising VLCDR1, VLCDR2, and VLCDR3 amino acid sequences derived from SEQ ID NO: 80;
(d) a heavy chain variable region (VH) comprising VHCDR1, VHCDR2, and VHCDR3 amino acid sequences derived from SEQ ID NO: 77 and a light chain variable region (VL) comprising VLCDR1, VLCDR2, and VLCDR3 amino acid sequences derived from SEQ ID NO: 80;
(e) a heavy chain variable region (VH) comprising VHCDR1, VHCDR2, and VHCDR3 amino acid sequences derived from SEQ ID NO: 79 and a light chain variable region (VL) comprising VLCDR1, VLCDR2, and VLCDR3 amino acid sequences derived from SEQ ID NO: 78;
(f) a heavy chain variable region (VH) comprising VHCDR1, VHCDR2, and VHCDR3 amino acid sequences derived from SEQ ID NO: 57 and a light chain variable region (VL) comprising VLCDR1, VLCDR2, and VLCDR3 amino acid sequences derived from SEQ ID NO: 58; or
(g) a heavy chain variable region (VH) comprising VHCDR1, VHCDR2, and VHCDR3 amino acid sequences derived from SEQ ID NO: 59 and a light chain variable region (VL) comprising VLCDR1, VLCDR2, and VLCDR3 amino acid sequences derived from SEQ ID NO: 60.

3. The monoclonal antibody or antibody fragment of claim 1 or 2, wherein the antibody or antibody fragment comprises clone-paired heavy and light chain CDR sequences from any of Tables 1-3.

4. The monoclonal antibody or antibody fragment of any one of claims 1-3, wherein the antibody or antibody fragment comprises clone-paired heavy chain and light chain variable sequences having, independently, at least 70%, 80%, or 90% identity to sequences from Tables 4, 6, and 7.

5. The monoclonal antibody or antibody fragment of any one of claims 1-4, wherein the antibody or antibody fragment comprises clone-paired heavy chain and light chain variable sequences each having at least 95% identity to sequences from Tables 4, 6, and 7.

6. The monoclonal antibody or antibody fragment of any one of claims 1-5, wherein the antibody or antibody fragment comprises clone-paired heavy chain and light chain variable sequences from Tables 4, 6, and 7.

7. The monoclonal antibody or antibody fragment of any one of claims 1-6, wherein said antibody or antibody fragment is a humanized antibody or antibody fragment.

8. The monoclonal antibody or antibody fragment of claim 7, wherein said antibody or antibody fragment comprises a heavy chain variable sequence and a light chain variable sequence each independently selected from Table 6 or 7 and having at least 70%, 80%, or 90% identity to the sequence from Table 6 or 7.

9. The monoclonal antibody or antibody fragment of claim 8, wherein said antibody or antibody fragment comprises a heavy chain variable sequence and a light chain variable sequence each independently selected from Table 6 or 7 and having at least 95% identity to the sequence from Table 6 or 7.

10. The monoclonal antibody or antibody fragment of claim 9, wherein said antibody or antibody fragment comprises a heavy chain variable sequence and a light chain variable sequence each independently selected from Table 6 or 7.

11. The monoclonal antibody or antibody fragment of any one of claims 7-10, wherein said antibody or antibody fragment comprises a heavy chain variable sequence at least 70%, 80%, or 90% identical to SEQ ID NO: 68, 70, 72, or 74.

12. The monoclonal antibody or antibody fragment of any one of claims 7-11, wherein said antibody or antibody fragment comprises a heavy chain variable sequence at least 95% identical to SEQ ID NO: 68, 70, 72, or 74.

13. The monoclonal antibody or antibody fragment of any one of claims 7-12, wherein said antibody or antibody fragment comprises a heavy chain variable sequence of SEQ ID NO: 68, 70, 72, or 74.

14. The monoclonal antibody or antibody fragment of any one of claims 7-13, wherein said antibody or antibody fragment comprises a light chain variable sequence at least 70%, 80%, or 90% identical to SEQ ID NO: 69, 71, or 73.

15. The monoclonal antibody or antibody fragment of any one of claims 7-14, wherein said antibody or antibody fragment comprises a light chain variable sequence at least 95% identical to SEQ ID NO: 69, 71, or 73.

16. The monoclonal antibody or antibody fragment of any one of claims 7-15, wherein said antibody or antibody fragment comprises a light chain variable sequence of SEQ ID NO: 69, 71, or 73.

17. The monoclonal antibody or antibody fragment of any one of claims 7-10, wherein said antibody or antibody fragment comprises a heavy chain variable sequence at least 70%, 80%, or 90% identical to SEQ ID NO: 77 or 79.

18. The monoclonal antibody or antibody fragment of any one of claims 7-10 and 17, wherein said antibody or antibody fragment comprises a heavy chain variable sequence at least 95% identical to SEQ ID NO: 77 or 79.

19. The monoclonal antibody or antibody fragment of any one of claims 7-10, 17, and 18, wherein said antibody or antibody fragment comprises a heavy chain variable sequence of SEQ ID NO: 77 or 79.

20. The monoclonal antibody or antibody fragment of any one of claims 7-10 and 17-19, wherein said antibody or antibody fragment comprises a light chain variable sequence at least 70%, 80%, or 90% identical to SEQ ID NO: 78 or 80.

21. The monoclonal antibody or antibody fragment of any one of claims 7-10 and 17-20, wherein said antibody or antibody fragment comprises a light chain variable sequence at least 95% identical to SEQ ID NO: 78 or 80.

22. The monoclonal antibody or antibody fragment of any one of claims 7-10 and 17-21, wherein said antibody or antibody fragment comprises a light chain variable sequence of SEQ ID NO: 78 or 80.

23. The monoclonal antibody or antibody fragment of any one of claims 1-22, wherein the antibody fragment is a monovalent scFv (single chain fragment variable) antibody, divalent scFv, Fab fragment, F(ab′)₂ fragment, F(ab′)3 fragment, Fv fragment, or single chain antibody.

24. The monoclonal antibody or antibody fragment of any one of claims 1-22, wherein said antibody is a chimeric antibody, bispecific antibody, or BiTE.

25. The monoclonal antibody or antibody fragment of any one of claims 1-24, wherein the antibody is capable of binding to a coronavirus spike protein.

26. The monoclonal antibody or antibody fragment of claim 25, wherein the coronavirus spike protein is from SARS-CoV, SARS-CoV-2, or MERS-CoV.

27. The monoclonal antibody or antibody fragment of any one of claims 1-26, wherein the antibody is capable of binding to the spike proteins from SARS-CoV, SARS-CoV-2, and MERS-CoV.

28. The monoclonal antibody or antibody fragment of any one of claims 1-27, wherein the antibody is an IgG antibody or a recombinant IgG antibody or antibody fragment.

29. The monoclonal antibody or antibody fragment of any one of claims 1-28, wherein the antibody or antibody fragment is fused to an imaging agent.

30. The monoclonal antibody or antibody fragment of any one of claims 1-28, wherein the antibody or antibody fragment is labeled.

31. The monoclonal antibody or antibody fragment of claim 30, wherein the label is a fluorescent label, an enzymatic label, or a radioactive label.

32. A monoclonal antibody or antibody fragment, which competes for binding to the same epitope of a coronavirus spike protein as the monoclonal antibody or an antibody fragment according to any one of claims 1-28.

33. A monoclonal antibody or antibody fragment that binds to an epitope on a coronavirus spike protein recognized by the monoclonal antibody or antibody fragment of any one of claims 1-28.

34. The monoclonal antibody or antibody fragment of claim 32 or 33, wherein the epitope is the apex of a coronavirus spike S2 domain.

35. The monoclonal antibody or antibody fragment of any one of claims 32-34, wherein the epitope is present within a portion of a coronavirus spike protein that corresponds to amino acids 980-1006 of SEQ ID NO: 67.

36. The monoclonal antibody or antibody fragment of any one of claims 32-35, wherein, when bound to a coronavirus spike protein, the monoclonal antibody binds to at least one residue of a coronavirus spike protein that corresponds to position 980, 983, 984, 985, 992, or 995 of SEQ ID NO: 67.

37. The monoclonal antibody or antibody fragment of any one of claims 32-36, wherein, when bound to a coronavirus spike protein, the monoclonal antibody binds to residues of a coronavirus spike protein that correspond to positions 980, 983, 984, 985, 992, and 995 of SEQ ID NO: 67.

38. The monoclonal antibody or antibody fragment of any one of claims 32-37, wherein, when bound to a coronavirus spike protein, the monoclonal antibody additionally binds to at least one residue of a coronavirus spike protein the corresponds to position 987, 988, or 990 of SEQ ID NO: 67.

39. The monoclonal antibody or antibody fragment of any one of claims 32-38, wherein the antibody is capable of binding to the spike proteins from SARS-CoV, SARS-CoV-2, and MERS-CoV.

40. An isolated nucleic acid encoding the antibody heavy and/or light chain variable region of the antibody or antibody fragment of any one of claims 1-28 and 32-39.

41. An expression vector comprising the nucleic acid of claim 40.

42. A hybridoma or engineered cell comprising a nucleic acid encoding an antibody or antibody fragment of any one of claims 1-28 and 32-39.

43. A hybridoma or engineered cell comprising a nucleic acid of claim 40.

44. A method of making the monoclonal antibody or antibody fragment of any one of claims 1-28 and 32-39, the method comprising culturing the hybridoma or engineered cell of claim 42 or 43 under conditions that allow expression of the antibody or antibody fragment and optionally isolating the antibody or antibody fragment from the culture.

45. A pharmaceutical formulation comprising one or more antibody or antibody fragment of any one of claims 1-39.

46. A pharmaceutical formulation comprising one or more expression vector encoding a first antibody or antibody fragment of any one of claims 1-28 and 32-39.

47. A method of reducing the likelihood of a pathogenic coronavirus infection in a patient at risk of contracting the pathogenic coronavirus, the method comprising delivering to the patient an antibody or antibody fragment of any one of claims 1-39 or a pharmaceutical formulation of claim 45 or 46.

48. The method of claim 47, further characterized as a method of preventing a SARS-CoV-2 infection in the patient.

49. The method of claim 47, wherein the patient has been exposed to a coronavirus.

50. The method of claim 47, wherein the antibody or antibody fragment is delivered to the patient prior to infection or after infection.

51. The method of any one of claims 47-50, wherein delivering comprises antibody or antibody fragment administration, or genetic delivery with an RNA or DNA sequence or vector encoding the antibody or antibody fragment.

52. A method of treating a patient infected with a pathogenic coronavirus, the method comprising delivering to the patient an antibody or antibody fragment of any one of claims 1-39 or a pharmaceutical formulation of claim 45 or 46.

53. The method of claim 52, wherein delivering comprises antibody or antibody fragment administration, or genetic delivery with an RNA or DNA sequence or vector encoding the antibody or antibody fragment.

54. The method of any one of claims claim 52-53, wherein the method reduces the viral load in the patient.

55. The method of any one of claims 52-54, further comprising administering at least a second antibody that binds to a distinct epitope relative to the antibody or antibody fragment of any one of claims 1-39.

56. A method of detecting a coronavirus infection in a patient, the method comprising:
(a) contacting a sample obtained from the patient with an antibody or antibody fragment of any one of claims 1-39; and
(b) detecting the coronavirus in the sample by detecting binding of the antibody or antibody fragment to a coronavirus antigen in the sample.

57. The method of claim 56, wherein the sample is a body fluid.

58. The method of claim 56 or 57, wherein said sample is blood, sputum, tears, saliva, mucous or serum, semen, cervical or vaginal secretions, amniotic fluid, placental tissues, urine, exudate, transudate, tissue scrapings or feces.

59. The method of any one of claims 56-58, wherein detecting comprises an ELISA, RIA, lateral flow assay or Western blot.

60. The method of any one of claims 56-59, further comprising performing steps (a) and (b) a second time and determining a change in coronavirus antigen levels as compared to the first assay.

61. The method of any one of claims 56-60, wherein the coronavirus is an emerging coronavirus.

62. The method of any one of claims 56-60, wherein the coronavirus has not yet been identified at the time the method is performed.

63. A method of determining an antigenic integrity, correct conformation and/or correct sequence of a coronavirus spike protein, the method comprising:
(a) contacting a sample comprising the coronavirus spike protein with a first antibody or antibody fragment of any one of claims 1-39; and
(b) determining antigenic integrity, correct conformation and/or correct sequence of the coronavirus spike protein by detecting binding of the first antibody or antibody fragment to the antigen.

64. The method of claim 63, wherein the sample comprises a recombinantly produced coronavirus spike protein.

65. The method of claim 63, wherein the sample comprises a vaccine formulation comprising the coronavirus spike protein.

66. The method of any one of claims 63-65, wherein detecting comprises an ELISA, RIA, biolayer interferometry (BLI), lateral flow assay or Western blot.

67. The method of any one of claims 63-66, further comprising performing steps (a) and (b) a second time to determine the antigenic stability of the coronavirus spike protein over time.

68. A method of detecting a coronavirus spike protein in an in vitro sample, the method comprising contacting the in vitro sample with an antibody or antibody fragment of any one of claims 1-39 and detecting the binding of the antibody or antibody fragment to the sample.

69. The method of claim 68, wherein the detecting is by flow cytometry, mass spectrometry, western blot, immunohistochemistry, ELISA, biolayer interferometry (BLI), or RIA.

70. An antibody or antibody fragment of any of claims 1-39 or a pharmaceutical formulation of claim 45 or 46, for use in treating or preventing a coronavirus infection in a patient.

71. Use of an antibody or antibody fragment of any of claims 1-39 or a pharmaceutical formulation of claim 45 or 46, in the manufacture of a medicament for treating or preventing a coronavirus infection in a patient.

72. An engineered protein comprising an engineered coronavirus S protein ectodomain that comprises a sequence at least 90% identical to: (a) positions 14-1208 of SEQ ID NO: 75 or 76; (b) positions 14-1160 of SEQ ID NO: 75 or 76; or (c) positions 319-1208 of SEQ ID NO: 75 or 76; wherein the engineered protein comprises the following substitutions relative to the sequence of SEQ ID NO: 75 or 76: F817P, A892P, A899P, A942P, K986P, V987P, and E1031R.

73. An engineered protein comprising an engineered coronavirus S protein ectodomain that comprises a sequence at least 90% identical to: (a) positions 14-1208 of SEQ ID NO: 75 or 76; (b) positions 14-1160 of SEQ ID NO: 75 or 76; or (c) positions 319-1208 of SEQ ID NO: 75 or 76; wherein the engineered protein comprises the following substitutions relative to the sequence of SEQ ID NO: 75 or 76: S383C, F817P, A892P, A899P, A942P, D985C, K986P, and V987P.

74. The engineered protein of claim 72 or 73, having at least 95% identity to positions 319-1208 of SEQ ID NO: 75 or 76.

75. The engineered protein of claim 72 or 73, comprising an engineered coronavirus S protein ectodomain having 95% identity to positions 14-1208 of SEQ ID NO: 75 or 76.

76. The engineered protein of any one of claims 72-75, wherein the engineered coronavirus S protein ectodomain comprises a mutation that eliminates the furin cleavage site.

77. The engineered protein of claim 76, wherein the mutation that eliminates the furin cleavage site comprises a GSAS substitution at positions 682-685.

78. The engineered protein of any one of claims 72-77, wherein the protein is fused or conjugated to a trimerization domain.

79. The engineered protein of claim 78, wherein the protein is fused to a trimerization domain.

80. The engineered protein of claim 78, wherein the a trimerization domain is positioned C-terminally relative to S protein ectodomain.

81. The engineered protein of claim 80, wherein the a trimerization domain comprises a T4 fibritin trimerization domain.

82. The engineered protein of any one of claims 72-81, wherein the protein is fused or conjugated to a transmembrane domain.

83. The engineered protein of claim 82, wherein the protein is fused to a transmembrane domain.

84. The engineered protein of claim 83, wherein the transmembrane domain comprises a coronavirus spike protein transmembrane domain.

85. The engineered protein of claim 83, wherein the transmembrane domain comprises a SARS-CoV-2 transmembrane domain.

86. An engineered coronavirus trimer comprising at least one subunit according to any one of claims 72-85.

87. A nucleic acid molecule comprising a nucleotide sequence that encodes an amino acid sequence of an engineered protein of any of claims 72-85.

88. A composition comprising an engineered protein of any of claims 72-85 bound to an antibody.

89. A method for detecting an antibody or antibody fragment that binds to an epitope on a coronavirus spike protein recognized by the monoclonal antibody or antibody fragment according to any one of claims 1-28 and 32-39, the method comprising contacting a sample comprising the engineered protein of any of claims 72-85 or the trimer of claim 86 with an antibody or antibody fragment and detecting binding of the antibody or antibody fragment to the engineered protein or trimer.

90. The method of claim 89, further wherein the sample comprises the 3A3 antibody or a fragment of the 3A3 antibody, and the method detects binding of the antibody or antibody fragment to the engineered protein or trimer to determine if the antibody or antibody fragment competes with 3A3 for binding to the engineered protein or trimer.

91. The method of claim 89 or 90, wherein the method detects an antibody or antibody fragment capable of binding to the spike proteins from SARS-CoV, SARS-CoV-2, and MERS-CoV.


SEQUENCE LISTING (92 sequences)

SEQ ID NO: 1 - protein, 8 aa, Artificial sequence
  Synthetic polypeptide sequence
GFNIKDTY

SEQ ID NO: 2 - protein, 8 aa, Artificial sequence
  Synthetic polypeptide sequence
IGPAIGNT

SEQ ID NO: 3 - protein, 13 aa, Artificial sequence
  Synthetic polypeptide sequence
ARYYGSSYYYFDY

SEQ ID NO: 4 - protein, 11 aa, Artificial sequence
  Synthetic polypeptide sequence
KSLLHSNGNTY

SEQ ID NO: 5 - protein, 3 aa, Artificial sequence
  Synthetic polypeptide sequence
RMS

SEQ ID NO: 6 - protein, 9 aa, Artificial sequence
  Synthetic polypeptide sequence
MQYLEYPLT

SEQ ID NO: 7 - protein, 8 aa, Artificial sequence
  Synthetic polypeptide sequence
GFTFSSYG

SEQ ID NO: 8 - protein, 8 aa, Artificial sequence
  Synthetic polypeptide sequence
ISSGGSYT

SEQ ID NO: 9 - protein, 13 aa, Artificial sequence
  Synthetic polypeptide sequence
ARDGYGNYVGMDY

SEQ ID NO: 10 - protein, 5 aa, Artificial sequence
  Synthetic polypeptide sequence
SSVSY

SEQ ID NO: 11 - protein, 3 aa, Artificial sequence
  Synthetic polypeptide sequence
STS

SEQ ID NO: 12 - protein, 9 aa, Artificial sequence
  Synthetic polypeptide sequence
QQWSSNSYT

SEQ ID NO: 13 - protein, 8 aa, Artificial sequence
  Synthetic polypeptide sequence
GYTFTSYW

SEQ ID NO: 14 - protein, 8 aa, Artificial sequence
  Synthetic polypeptide sequence
IDPSNSET

SEQ ID NO: 15 - protein, 6 aa, Artificial sequence
  Synthetic polypeptide sequence
ARLGRY

SEQ ID NO: 16 - protein, 6 aa, Artificial sequence
  Synthetic polypeptide sequence
QNVGAN

SEQ ID NO: 17 - protein, 3 aa, Artificial sequence
  Synthetic polypeptide sequence
SAS

SEQ ID NO: 18 - protein, 13 aa, Artificial sequence
  Synthetic polypeptide sequence
QQYNSYPYTSGGG

SEQ ID NO: 19 - protein, 9 aa, Artificial sequence
  Synthetic polypeptide sequence
FNIKDTYIH

SEQ ID NO: 20 - protein, 15 aa, Artificial sequence
  Synthetic polypeptide sequence
WIGRIGPAIGNTIYA

SEQ ID NO: 21 - protein, 13 aa, Artificial sequence
  Synthetic polypeptide sequence
ARYYGSSYYYFDY

SEQ ID NO: 22 - protein, 13 aa, Artificial sequence
  Synthetic polypeptide sequence
KSLLHSNGNTYLY

SEQ ID NO: 23 - protein, 11 aa, Artificial sequence
  Synthetic polypeptide sequence
LLIYRMSNLAS

SEQ ID NO: 24 - protein, 8 aa, Artificial sequence
  Synthetic polypeptide sequence
MQYLEYPL

SEQ ID NO: 25 - protein, 9 aa, Artificial sequence
  Synthetic polypeptide sequence
FTFSSYGMS

SEQ ID NO: 26 - protein, 14 aa, Artificial sequence
  Synthetic polypeptide sequence
WVATISSGGSYTYY

SEQ ID NO: 27 - protein, 12 aa, Artificial sequence
  Synthetic polypeptide sequence
RDGYGNYVGMDY

SEQ ID NO: 28 - protein, 7 aa, Artificial sequence
  Synthetic polypeptide sequence
SSVSYMH

SEQ ID NO: 29 - protein, 11 aa, Artificial sequence
  Synthetic polypeptide sequence
LWIYSTSNLAS

SEQ ID NO: 30 - protein, 8 aa, Artificial sequence
  Synthetic polypeptide sequence
QQWSSNSY

SEQ ID NO: 31 - protein, 9 aa, Artificial sequence
  Synthetic polypeptide sequence
YTFTSYWMH

SEQ ID NO: 32 - protein, 14 aa, Artificial sequence
  Synthetic polypeptide sequence
WVGMIDPSNSETRL

SEQ ID NO: 33 - protein, 5 aa, Artificial sequence
  Synthetic polypeptide sequence
RLGRY

SEQ ID NO: 34 - protein, 8 aa, Artificial sequence
  Synthetic polypeptide sequence
QNVGANVA

SEQ ID NO: 35 - protein, 11 aa, Artificial sequence
  Synthetic polypeptide sequence
ALIYSASYRYS

SEQ ID NO: 36 - protein, 8 aa, Artificial sequence
  Synthetic polypeptide sequence
QQYNSYPY

SEQ ID NO: 37 - protein, 7 aa, Artificial sequence
  Synthetic polypeptide sequence
GFNIKDT

SEQ ID NO: 38 - protein, 6 aa, Artificial sequence
  Synthetic polypeptide sequence
GPAIGN

SEQ ID NO: 39 - protein, 11 aa, Artificial sequence
  Synthetic polypeptide sequence
YYGSSYYYFDY

SEQ ID NO: 40 - protein, 16 aa, Artificial sequence
  Synthetic polypeptide sequence
RSSKSLLHSNGNTYLY

SEQ ID NO: 41 - protein, 7 aa, Artificial sequence
  Synthetic polypeptide sequence
RMSNLAS

SEQ ID NO: 42 - protein, 9 aa, Artificial sequence
  Synthetic polypeptide sequence
MQYLEYPLT

SEQ ID NO: 43 - protein, 7 aa, Artificial sequence
  Synthetic polypeptide sequence
GFTFSSY

SEQ ID NO: 44 - protein, 6 aa, Artificial sequence
  Synthetic polypeptide sequence
SSGGSY

SEQ ID NO: 45 - protein, 11 aa, Artificial sequence
  Synthetic polypeptide sequence
DGYGNYVGMDY

SEQ ID NO: 46 - protein, 10 aa, Artificial sequence
  Synthetic polypeptide sequence
SASSSVSYMH

SEQ ID NO: 47 - protein, 7 aa, Artificial sequence
  Synthetic polypeptide sequence
STSNLAS

SEQ ID NO: 48 - protein, 9 aa, Artificial sequence
  Synthetic polypeptide sequence
QQWSSNSYT

SEQ ID NO: 49 - protein, 7 aa, Artificial sequence
  Synthetic polypeptide sequence
GYTFTSY

SEQ ID NO: 50 - protein, 6 aa, Artificial sequence
  Synthetic polypeptide sequence
DPSNSE

SEQ ID NO: 51 - protein, 4 aa, Artificial sequence
  Synthetic polypeptide sequence
LGRY

SEQ ID NO: 52 - protein, 13 aa, Artificial sequence
  Synthetic polypeptide sequence
KASQNVGANVAWY

SEQ ID NO: 53 - protein, 7 aa, Artificial sequence
  Synthetic polypeptide sequence
SASYRYS

SEQ ID NO: 54 - protein, 9 aa, Artificial sequence
  Synthetic polypeptide sequence
QQYNSYPYT

SEQ ID NO: 55 - protein, 469 aa, Artificial sequence
  Synthetic polypeptide sequence
  SIGNAL (1)..(19)
MGWSCIILFLVATATGVHSQVQLQQSGAELLKPGASVKLSCTASGFNIKDTYIHWLKQRPEQGLEWIGRIGPAIGNTIYAPKFQGKATITTDTSSNTAYLQLSSLTSEDTAVYYCARYYGSSYYYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 238 aa, Artificial sequence
  Synthetic polypeptide sequence
  SIGNAL (1)..(19)
MGWSCIILFLVATATGVHSDIVMTQSAPSVPVTPGESVSISCRSSKSLLHSNGNTYLYWFLQRPGQSPQLLIYRMSNLASGVPDRFSGSGSGTAFTLRISRVEAEDVGVYYCMQYLEYPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 469 aa, Artificial sequence
  Synthetic polypeptide sequence
  SIGNAL (1)..(19)
MGWSCIILFLVATATGVHSEVQVVESGGGLVQPGGSRKLSCAASGFTFSSYGMSWVRQTPDKRLEWVATISSGGSYTYYPDSVKGRFTISRDNAKNNLYLQMSSLKSEDTAMYYCARDGYGNYVGMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 232 aa, Artificial sequence
  Synthetic polypeptide sequence
  SIGNAL (1)..(19)
MGWSCIILFLVATATGVHSDIVLTQSPAIMSASPGEKVTITCSASSSVSYMHWFQQKPGTSPKLWIYSTSNLASGVPARFSGSGSGTSYSLTISSMEAEDAATYYCQQWSSNSYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 462 aa, Artificial sequence
  Synthetic polypeptide sequence
  SIGNAL (1)..(19)
MGWSCIILFLVATATGVHSQVQLQQSGAELAKPGASVKMSCKASGYTFTSYWMHWVKQRPGQGLEWVGMIDPSNSETRLNQKFKDKATLNVDKSSNTAYMQLSSLTSEDSAVYYCARLGRYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 233 aa, Artificial sequence
  Synthetic polypeptide sequence
  SIGNAL (1)..(19)
MGWSCIILFLVATATGVHSDIVMTQSHKFMSTSVGDRVSITCKASQNVGANVAWYQQKPGQSPKALIYSASYRYSGVPDRFTGSGSGTDFTLTISNVQSEDLAEYFCQQYNSYPYTSGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 254 aa, Artificial sequence
  Synthetic polypeptide sequence
  SIGNAL (1)..(23)
MRPTWAWWLFLVLLLALWAPARGQVQLQQSGAELLKPGASVKLSCTASGFNIKDTYIHWLKQRPEQGLEWIGRIGPAIGNTIYAPKFQGKATITTDTSSNTAYLQLSSLTSEDTAVYYCARYYGSSYYYFDYWGQGTTLTVSSASTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGKGLEVLFQ

SEQ ID NO: 62 - protein, 239 aa, Artificial sequence
  Synthetic polypeptide sequence
  SIGNAL (1)..(19)
MGWSCIILFLVATATGVHSDIVMTQSAPSVPVTPGESVSISCRSSKSLLHSNGNTYLYWFLQRPGQSPQLLIYRMSNLASGVPDRFSGSGSGTAFTLRISRVEAEDVGVYYCMQYLEYPLTFGAGTKLELKRTADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 63 - protein, 254 aa, Artificial sequence
  Synthetic polypeptide sequence
  SIGNAL (1)..(23)
MRPTWAWWLFLVLLLALWAPARGEVQVVESGGGLVQPGGSRKLSCAASGFTFSSYGMSWVRQTPDKRLEWVATISSGGSYTYYPDSVKGRFTISRDNAKNNLYLQMSSLKSEDTAMYYCARDGYGNYVGMDYWGQGTSVTVSSASTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGKGLEVLFQ

SEQ ID NO: 64 - protein, 233 aa, Artificial sequence
  Synthetic polypeptide sequence
  SIGNAL (1)..(19)
MGWSCIILFLVATATGVHSDIVLTQSPAIMSASPGEKVTITCSASSSVSYMHWFQQKPGTSPKLWIYSTSNLASGVPARFSGSGSGTSYSLTISSMEAEDAATYYCQQWSSNSYTFGGGTKLEIKRTADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 65 - protein, 247 aa, Artificial sequence
  Synthetic polypeptide sequence
  SIGNAL (1)..(23)
MRPTWAWWLFLVLLLALWAPARGQVQLQQSGAELAKPGASVKMSCKASGYTFTSYWMHWVKQRPGQGLEWVGMIDPSNSETRLNQKFKDKATLNVDKSSNTAYMQLSSLTSEDSAVYYCARLGRYWGQGTTLTVSSASTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGKGLEVLFQ

SEQ ID NO: 66 - protein, 234 aa, Artificial sequence
  Synthetic polypeptide sequence
  SIGNAL (1)..(19)
MGWSCIILFLVATATGVHSDIVMTQSHKFMSTSVGDRVSITCKASQNVGANVAWYQQKPGQSPKALIYSASYRYSGVPDRFTGSGSGTDFTLTISNVQSEDLAEYFCQQYNSYPYTSGGGTKLEIKRTADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 67 - protein, 1288 aa, Artificial sequence
  Synthetic polypeptide sequence
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGHHHHHHHHSAWSHPQFEKGGGSGGGGSGGSAWSHPQFEK

SEQ ID NO: 68 - protein, 120 aa, Artificial Sequence
  Synthetic polypeptide sequence
QVQLVQSGAEVLKPGASVKLSCKASGFNIKDTYIHWLKQAPGQRLEWIGRIGPAIGNTIYAPKFQGKATITTDTSASTAYLELSSLRSEDTAVYYCARYYGSSYYYFDYWGQGTTVTVSS

SEQ ID NO: 69 - protein, 112 aa, Artificial Sequence
  Synthetic polypeptide sequence
DIVMTQSPLSVPVTPGEPVSISCRSSKSLLHSNGNTYLYWFLQKPGQSPQLLIYRMSNLASGVPDRFSGSGSGTAFTLKISRVEAEDVGVYYCMQYLEYPLTFGAGTKLEIK

SEQ ID NO: 70 - protein, 120 aa, Artificial Sequence
  Synthetic polypeptide sequence
QVQLVQSGAEVKKPGASVKVSCKASGFNIKDTYIHWVRQAPGQRLEWIGRIGPAIGNTIYAPKFQGRVTITRDTSASTAYMELSSLRSEDTAVYYCARYYGSSYYYFDYWGQGTTVTVSS

SEQ ID NO: 71 - protein, 112 aa, Artificial Sequence
  Synthetic polypeptide sequence
DIVMTQSPLSLPVTPGEPASISCRSSKSLLHSNGNTYLYWFLQKPGQSPQLLIYRMSNLASGVPDRFSGSGSGTAFTLKISRVEAEDVGVYYCMQYLEYPLTFGAGTKLEIK

SEQ ID NO: 72 - protein, 120 aa, Artificial Sequence
  Synthetic polypeptide sequence
QVQLVQSGAEVKKPGASVKVSCKASGFNIKDTYMHWVRQAPGQRLEWIGRIGPAIGNTIYSQKFQGRVTITRDTSASTAYMELSSLRSEDTAVYYCARYYGSSYYYFDYWGQGTTVTVSS

SEQ ID NO: 73 - protein, 112 aa, Artificial Sequence
  Synthetic polypeptide sequence
DIVMTQSPLSLPVTPGEPASISCRSSKSLLHSNGNTYLYWFLQKPGQSPQLLIYRGSNLASGVPDRFSGSGSGTAFTLKISRVEAEDVGVYYCMQYLEYPLTFGAGTKLEIK

SEQ ID NO: 74 - protein, 120 aa, Artificial Sequence
  Synthetic polypeptide sequence
QVQLVQSGAEVKKPGASVKVSCKASGFNIKDTYIHWVRQAPGQRLEWIGRIGPAIGNTIYSQKFQGRVTITRDTSASTAYMELSSLRSEDTAVYYCARYYGSSYYYFDYWGQGTTVTVSS

SEQ ID NO: 75 - protein, 5 aa, Artificial Sequence
  SARS-CoV-2 ectodomain with trimerization domain and tags at the C-terminus
AAAAA

SEQ ID NO: 76 - protein, 6 aa, Artificial Sequence
  SARS-CoV-2 ectodomain consensus sequence for wild-type and variants
AAAAAA

SEQ ID NO: 77 - protein, 120 aa, Artificial Sequence
  Synthetic polypeptide sequence
QVQLVQSGADVLKPGASVKLSCKASGFNIKNTYIHWLKQAPGQRLEWIGRIGPAIGNTIYAPKFQGKATITTDTSASTAYLELSSLRSEDTAVYYCARYYGSSYYYLDYWGQGTTVTVSS

SEQ ID NO: 78 - protein, 112 aa, Artificial Sequence
  Synthetic polypeptide sequence
DIVMTQSPLSVPVTPGEPVSISCRSSKSLHHSNGNTYLYWFLQKPGQSPQLLIYRMSNLASGVPDRFSGSGNGTAFTLKISRVEAEDVGVYYCMQYLEYPLTFDAGTKLEIK

SEQ ID NO: 79 - protein, 120 aa, Artificial Sequence
  Synthetic polypeptide sequence
QVQLVQSGAEVLKPGASVKLSCKASGFNIKDTYIHWLKQAPGQRLEWIGRIGPAIGNTIYAPKFQGKATITTDTSASTAYLELSSLRSEDTAVYYCARYYGYAYYYFDYWGQGTTVTVSS

SEQ ID NO: 80 - protein, 112 aa, Artificial Sequence
  Synthetic polypeptide sequence
DIVMTQSPLSVPVTPGEPVSISCRSSKSLLHSNGNTYLYWFLQKPGQSPQLLIARMSTLASGVPDRFSGSGSGTAFTLKISRVEAEDVGVYYCMQYLEYPLTFGAGTKLEIK

SEQ ID NO: 81 - protein, 8 aa, Artificial Sequence
  Synthetic polypeptide sequence
GFNIKNTY

SEQ ID NO: 82 - protein, 13 aa, Artificial Sequence
  Synthetic polypeptide sequence
ARYYGSSYYYLDY

SEQ ID NO: 83 - protein, 11 aa, Artificial Sequence
  Synthetic polypeptide sequence
KSLHHSNGNTY

SEQ ID NO: 84 - protein, 13 aa, Artificial Sequence
  Synthetic polypeptide sequence
ARYYGYAYYYFDY

SEQ ID NO: 85 - protein, 9 aa, Artificial Sequence
  Synthetic polypeptide sequence
FNIKNTYIH

SEQ ID NO: 86 - protein, 13 aa, Artificial Sequence
  Synthetic polypeptide sequence
KSLHHSNGNTYLY

SEQ ID NO: 87 - protein, 11 aa, Artificial Sequence
  Synthetic polypeptide sequence
LLIARMSTLAS

SEQ ID NO: 88 - protein, 7 aa, Artificial Sequence
  Synthetic polypeptide sequence
GFNIKNT

SEQ ID NO: 89 - protein, 11 aa, Artificial Sequence
  Synthetic polypeptide sequence
YYGSSYYYLDY

SEQ ID NO: 90 - protein, 16 aa, Artificial Sequence
  Synthetic polypeptide sequence
RSSKSLHHSNGNTYLY

SEQ ID NO: 91 - protein, 11 aa, Artificial Sequence
  Synthetic polypeptide sequence
YYGYAYYYFDY

SEQ ID NO: 92 - protein, 7 aa, Artificial Sequence
  Synthetic polypeptide sequence
RMSTLAS